(19) United States
(12) Patent Application Publication
(10) Pub. No.: US 2024/0182423 A1
(43) Pub. Date: Jun. 6, 2024

(54) 4-AMINOQUINOLINES FOR TREATMENT OF MULTIDRUG RESISTANT MALARIA

(71) Applicants: United States Government as represented by the Department of Veterans Affairs, Washington, DC (US); Oregon Health & Science University, Portland, OR (US)

(72) Inventors: Mason J. Handford, Portland, OR (US); Michael Riscoe, Tualatin, OR (US)

(21) Appl. No.: 18/384,695

(22) Filed: Oct. 27, 2023

Related U.S. Application Data
(60) Provisional application No. 63/381,484, filed on Oct. 28, 2022.

(51) Int. Cl. C07D 215/46; A61P 33/06

(52) U.S. Cl. CPC C07D 215/46; A61P 33/06

(57) ABSTRACT

The present invention concerns substituted N-(4-(piperidin-4-yl)phenyl)quinolin-4-amine and N-(4-(piperazin-1-yl)phenyl)quinolin-4-amine compounds having the general Formula (I), below, and useful in the treatment of malaria, including Multidrug Resistant Malaria. This abstract is intended as a scanning tool for purposes of searching in the particular art and is not intended to be limiting of the present invention.

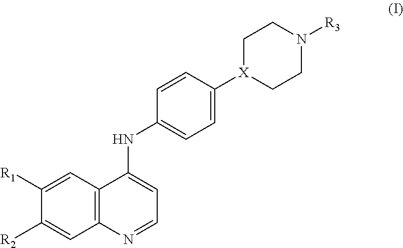

### CROSS-REFERENCE TO RELATED APPLICATIONS

This application claims the benefit of U.S. Application No. 63/381,484, filed on Oct. 28, 2022, the contents of which are incorporated herein by reference in their entirety.

### STATEMENT OF GOVERNMENT SUPPORT

This invention was made with government support under grant numbers 5R01AI100569-05 and R01AI141412 awarded by the National Institutes of Health. The government has certain rights in this invention.

### BACKGROUND

Malaria caused over 400,000 deaths in 2019, and infected over 220 million people worldwide (World malaria report 2021; World Health Organization, Geneva, 2021). The disease is caused by the infection of five species of the genus Plasmodium, with the majority of malaria fatalities caused by the single species, Plasmodium falciparum (Pf). 1 Plasmodium has a robust resistome that has been shown to rapidly develop resistance to current and developing therapeutics (Pasupureddy, R.; Atul; Seshadri, S.; Pande, V.; Dixit, R.; Pandey, K. C. Current scenario and future strategies to fight artemisinin resistance. Parasitol Res 2019, 118 (1), 29-42. DOI: 10.1007/s00436-018-6126-x; and Carolino, K.; Winzeler, E. A. The antimalarial resistome—finding new drug targets and their modes of action. Curr Opin Microbiol 2020, 57, 49-55. DOI: 10.1016/j.mib.2020.06.004).

Resistance to one or more chemical therapies employed to combat the disease has emerged as a serious issue, and has necessitated the use of combination therapies (Burrows et al., Antimalarial drug discovery—the path towards eradication. Parasitology 2014, 141 (1), 128-139. DOI:10.1017/50031182013000826. Burrows, J. N.; van Huijsduijnen, R. H.; Mohrle, J. J.; Oeuvray, C.; Wells, T. N. Designing the next generation of medicines for malaria control and eradication. Malar J 2013, 12, 187. DOI: 10.1186/1475-2875-12-187). Recently, resistance to the front-line combination therapy dihydroartemisinin-piperaquine been discovered in Southeast Asia, and East Africa (Carolino, K.; Winzeler, E. A. The antimalarial resistome—finding new drug targets and their modes of action. Curr Opin Microbiol 2020, 57, 49-55. DOI: 10.1016/j.mib.2020.06.004; and Burroughs, above). Therefore, the development of novel therapeutics with the ability to combat emerging resistance, is essential to address this threat.

4-Aminoquinoline compounds are known in the art, including those described in the article titled 4-Aminoquinoline, Lowe et al., Arzneimittel-Forschung, Vol. 16, Issue 10, pp. 1306-10, 1966; the article Search for new antiparasitic agents. 10. Synthesis, toxicity, and antimalarial effect of some nitrogen-containing heterocycles with 4-(4-alkylpiperazin-1-yl) phenylamino substituents, Mikhailitsyn et al., Meditsinskaya Parazitologiya i Parazitarnye Bolezni, Issue 1, Pages 50-3, 1992; U.S. Patent Application Publication No. 2003/0225078 (Wurster et al., Orion Corp.); PCT Publication No. WO 2004/067513 (Hoglund et al., Oy Juvantia Pharma Ltd.); Structure-Activity Relationship of Quinoline Derivatives as Potent and Selective α2C-Adrenoceptor Antagonists, Hogeland et al., Journal of Medicinal Chemistry, Volume 49, Issue 21, pages 6351-6363, 2006; QSAR study of a series of quinoline derivatives active on the alpha2 adrenergic receptor subtypes, Revue Roumaine de Chimie, Volume 54, Issue 8, pages 651-657, 2009; 4-Anilino-6-phenyl-quinoline inhibitors of mitogen activated protein kinase-activated protein kinase 2 (MK2), Olsson et al., Bioorganic & Medicinal Chemistry Letters, Volume 20, Issue 16, pages 4738-4740, 2010; A QSAR study using MTD method and Dragon descriptors for a series of selective ligands of α2C adrenoceptor, Borota et al., European Journal of Medicinal Chemistry, Volume 46, Issue 3, pages 877-884, 2011; Anti-prion activities and drug-like potential of functionalized quinacrine analogs with basic phenyl residues at the 9-amino position, Nguyen et al., European Journal of Medicinal Chemistry, Volume 46, Issue 7, pages 2917-2929, 2011; Functionalized acridin-9-yl phenylamines protected neuronal HT22 cells from glutamate-induced cell death by reducing intracellular levels of free radical species, Nguyen et al., Bioorganic & Medicinal Chemistry Letters, Volume 24, Issue 7, pages 1830-1838, 2014; and Optimized and convergent synthesis of potent anti-malarial aminoquinoline compounds: easy access to analogs, Le Fur et al., Heterocyclic Communications, Volume 16, Issue 4-6, pages 235-239, 2010.

One aminoquinoline, amodiaquine (AQ) exhibited superb activity against chloroquine-resistant (CQ-resistant) strains of Plasmodium falciparum, but was metabolically unstable, leading to toxic metabolites that ultimately reduced its widespread use.

There remains a need for improved pharmaceutical agents and methods for treating multi-drug resistant malaria.

### SUMMARY

An embodiment provides a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof:

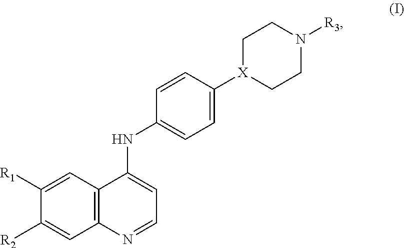

wherein: R₁ is selected from the group of H, and halogen; R₂ is selected from the group of H, halogen, and halomethyl; and R₃ is selected from the group of H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; X is selected from the group of N and C; with the proviso that, when R₁ is H, R₂ is not H; with the proviso that, when R₂ is H, R₁ is not H; and with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; and 6-bromo-N-(4-(4-ethylpiperazin-1-yl)phenyl)quinolin-4-amine; or a pharmaceutically acceptable salt thereof.

In one embodiment, provided are compounds selected from:

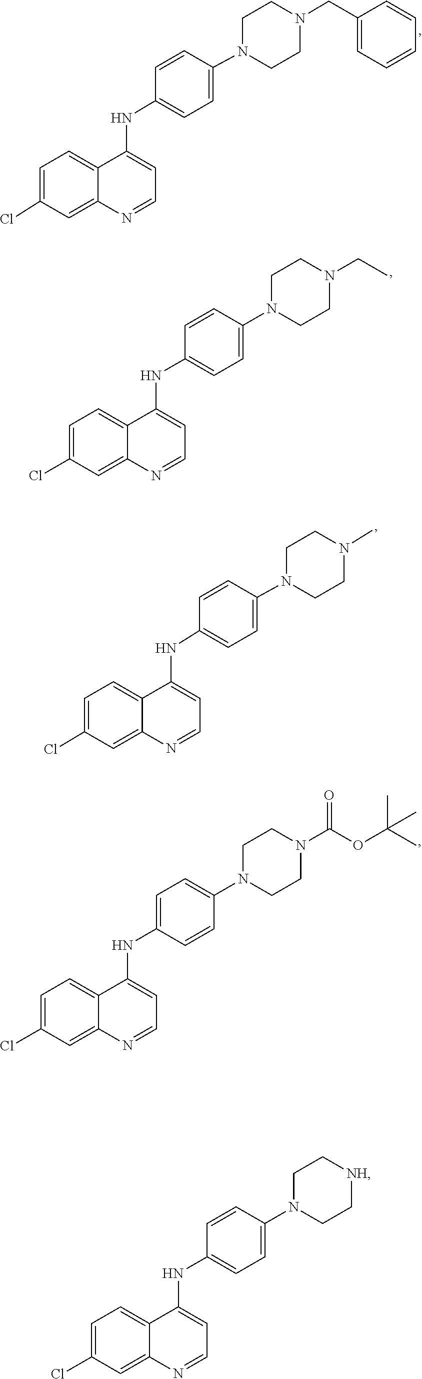 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1]

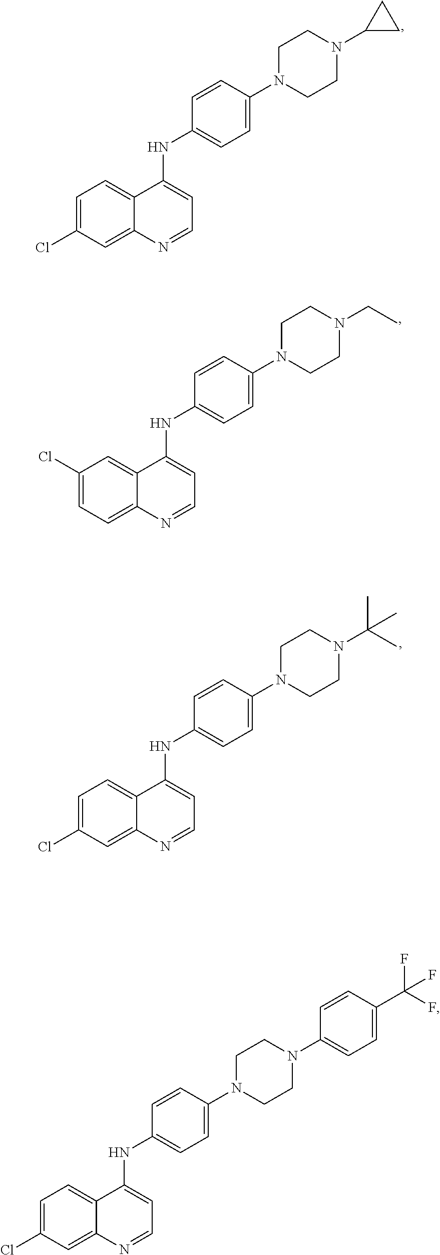 [SMILES: Clc1ccc2c(Nc3ccc(N4CCN(C5CC5)CC4)cc3)ccnc2c1 ; CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1]

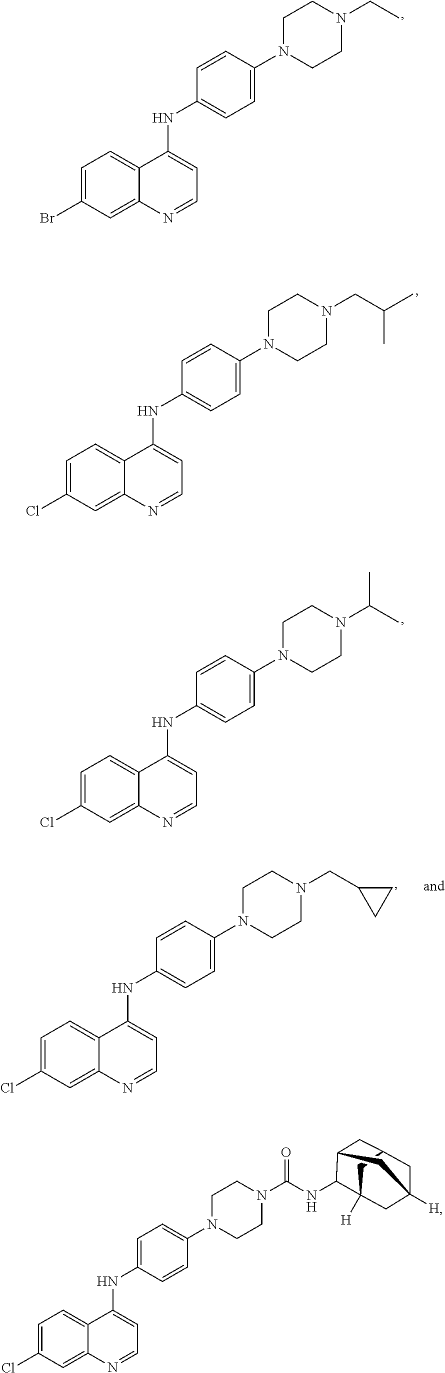

or a pharmaceutically acceptable salt thereof.

In one embodiment, provided are pharmaceutical compositions comprising a pharmaceutically acceptable carrier or excipient and a pharmaceutically effective amount of a disclosed compound; or a pharmaceutically acceptable salt thereof.

In one embodiment, provided are uses of a disclosed compound, or a pharmaceutically acceptable salt thereof, in the preparation of a medicament.

In one embodiment, provided are uses of a disclosed compound, or a pharmaceutically acceptable salt thereof, in the preparation of a medicament for the treatment of malaria.

In one embodiment, provided are methods of treating malaria in a subject in need thereof, the method comprising administering to the subject a pharmaceutically effective amount of a disclosed compound; or a pharmaceutically acceptable salt thereof.

In one embodiment, provided are methods method of treating malaria in a subject in need thereof, the method comprising administering to the subject a pharmaceutically effective amount of a compound of Formula (I):

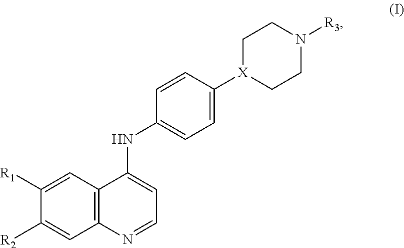

wherein: R₁ is selected from the group of H, and halogen; R₂ is selected from the group of H, halogen, and halomethyl; R₃ is selected from the group of H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; X is selected from the group of N and C; with the proviso that, when R₁ is H, R₂ is not H; with the proviso that, when R₂ is H, R₁ is not H; or a pharmaceutically acceptable salt thereof.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings.

FIG. 1 shows representative mean plasma concentration-time profiles of ADC-028 after PO doses at 5 mg/kg in male CD1 mice (N=3).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Provided herein are compounds and pharmaceutical compositions useful against Plasmodium falciparum, including multi-drug-resistant strains.

One embodiment provides a compound of Formula (I), above, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, wherein: R₁ is selected from the group of H, and halogen; R₂ is selected from the group of H, halogen, and halomethyl; and R₃ is selected from the group of H, C₁-C₇ straight or branched alkyl, C₃-C₆ cycloalkyl, —CH₂—C₃-C₆ cycloalkyl, and benzyl; X is selected from the group of N and C; with the proviso that, when R₁ is H, R₂ is not H; with the proviso that, when R₂ is H, R₁ is not H; and with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; and 6-bromo-N-(4-(4-ethylpiperazin-1-yl)phenyl)quinolin-4-amine; or a pharmaceutically acceptable salt thereof.

Another embodiment provides a compound of Formula (I), above, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, wherein: R₁ is selected from the group of H, and C₁; R₂ is selected from the group of H, Cl, and fluoromethyl; and R₃ is selected from the group of H, C₁-C₇ straight or branched alkyl, C₃-C₆ cycloalkyl, —CH₂—C₃-C₆ cycloalkyl, and benzyl; X is selected from the group of N and C; with the proviso that, when R₁ is H, R₂ is not H; and with the proviso that, when R₂ is H, R₁ is not H; and with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; and 6-bromo-N-(4-(4-ethylpiperazin-1-yl)phenyl)quinolin-4-amine; or a pharmaceutically acceptable salt thereof.

A different embodiment provides a compound of Formula (I), above, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, wherein: R₁ is H; R₂ is selected from the group of C₁ and fluoromethyl; and R₃ is selected from the group of H, C₁-C₇ straight or branched alkyl, C₃-C₆ cycloalkyl, —CH₂—C₃-C₆ cycloalkyl, and benzyl; X is selected from the group of N and C; with the proviso that, when R₁ is H, R₂ is not H; with the proviso that, when R₂ is H, R₁ is not H; and with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; and 6-bromo-N-(4-(4-ethylpiperazin-1-yl)phenyl)quinolin-4-amine; or a pharmaceutically acceptable salt thereof.

A different embodiment provides a compound of Formula (I), above, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, wherein: R₁ is H; R₂ is selected from the group of Cl and CF₃; and R₃ is selected from the group of H, C₁-C₇ straight or branched alkyl, C₃-C₆ cycloalkyl, —CH₂—C₃-C₆ cycloalkyl, and benzyl; and X is selected from the group of N and C; with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; and 7-chloro-N-(4-(1-methylpiperidin-4-yl)phenyl)quinolin-4-amine; or a pharmaceutically acceptable salt thereof.

A separate embodiment provides a compound of Formula (II), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof:

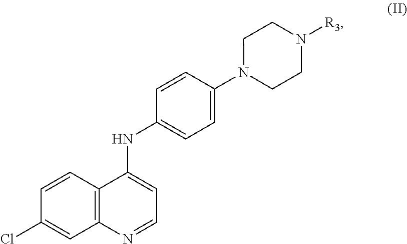

wherein R₃ is selected from the group of H, C₁-C₇ straight or branched alkyl, C₃-C₆ cycloalkyl, —CH₂—C₃-C₆ cycloalkyl, and benzyl; with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; and 7-chloro-N-(4-(1-methylpiperidin-4-yl)phenyl)quinolin-4-amine; or a pharmaceutically acceptable salt thereof.

A further embodiment provides a compound of Formula (II), above, wherein R₃ is selected from the group of ethyl, n-propyl, isopropyl (—CH(CH₃)₂), tert-butyl (—C(CH₃)₃), isobutyl (—CH₂—CH(CH₃)₂), sec-butyl (—CH(CH₃)—CH₂—CH₃), n-pentyl, pentan-2-yl (—CH(CH₃)CH₂CH₂CH₃), 2-methylbutyl (—CH₂CH(CH₃)CH₂CH₃), isopentyl (CH₂CH₂CH(CH₃)₂), 3-methylbutan-2-yl (CH(CH₃)CH(CH₃)₂), n-hexyl (CH₂CH₂CH₂CH₂CH₂CH₃), isohexyl (CH₂CH₂CH₂CH(CH₃)₂), t-hexyl (C(CH₃)₂CH₂CH₂CH₃), sec-hexyl (—CH(CH₃)CH₂CH₂CH₂CH₃), 2-methylpentyl (CH₂CH(CH₃)CH₂CH₂CH₃), 3-methylpentyl (CH₂CH₂CH(CH₃)CH₂CH₃), n-heptyl (—CH₂CH₂CH₂CH₂CH₂CH₂CH₃), 5-methylhexyl (—CH₂CH₂CH₂CH₂CH(CH₃)₂), t-heptyl (—C(CH₃)₂CH₂CH₂CH₂CH₃), sec-heptyl (—CH(CH₃)CH₂CH₂CH₂CH₂CH₃), and iso-heptyl ((—CH₂CH₂CH₂CH₂CH(CH₃)₂); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (II), above, wherein R₃ is selected from the group of H, ethyl, n-propyl, isopropyl (—CH(CH₃)₂), tert-butyl (—C(CH₃)₃), isobutyl (—CH₂—CH(CH₃)₂), and sec-butyl (—CH(CH₃)—CH₂—CH₃); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (II), above, wherein R₃ is selected from the group of H, C₁-C₄ straight or branched alkyl, C₃-C₆ cycloalkyl, —CH₂—C₃-C₆ cycloalkyl, and benzyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; and 7-chloro-N-(4-(1-methylpiperidin-4-yl)phenyl)quinolin-4-amine; or a pharmaceutically acceptable salt thereof.

Still another embodiment provides a compound of Formula (II), above, wherein R₃ is selected from the group of H, C₁-C₄ straight or branched alkyl, C₃-C₆ cycloalkyl, and benzyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof with the proviso that the compound is not selected from the group of N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; and 7-chloro-N-(4-(1-methylpiperidin-4-yl)phenyl)quinolin-4-amine; or a pharmaceutically acceptable salt thereof.

Yet another embodiment provides a compound of Formula (II), above, wherein R₃ is C₁-C₄ straight or branched alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; with the proviso that the compound is not selected from the group N-(4-(4-butylpiperazin-1-yl)phenyl)-7-chloroquinolin-4-amine; and 7-chloro-N-(4-(4-methylpiperazin-1-yl)phenyl)quinolin-4-amine; or a pharmaceutically acceptable salt thereof.

Another embodiment provides a compound of Formula (III):

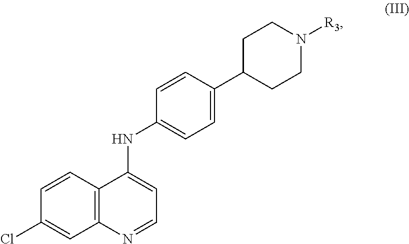

wherein R₃ is selected from the group of H and C₂-C₇ straight or branched alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

An embodiment herein provides a method for treating malaria in a subject, the method comprising administering to the human in need thereof a pharmaceutically effective amount of a compound of Formula (I),

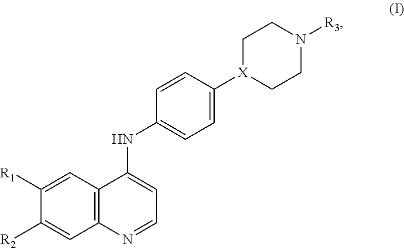

wherein: R₁ is selected from the group of H, and halogen; R₂ is selected from the group of H, halogen, and halomethyl; and R₃ is selected from the group of H, C₁-C₇ straight or branched alkyl, C₃-C₆ cycloalkyl, —CH₂—C₃-C₆ cycloalkyl, and benzyl; X is selected from the group of N and C; with the proviso that, when R₁ is H, R₂ is not H; and with the proviso that, when R₂ is H, R₁ is not H; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

An additional embodiment provides a compound of Formula (IV):

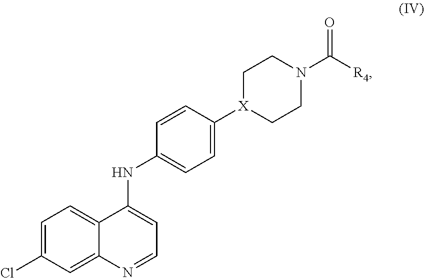

wherein: X is selected from the group of N and C; and R₄ is selected from the group of H, C₁-C₇ straight or branched alkyl, and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides the compound of Formula (IV), wherein X is N; and R₄ is selected from the group of H, C₁-C₇ straight or branched alkyl, and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides the compound of Formula (IV), wherein X is C; and R₄ is selected from the group of H, C₁-C₇ straight or branched alkyl, and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Still another embodiment provides the compound of Formula (IV), wherein X is N; and R₄ is C₁-C₇ straight or branched alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Yet another embodiment provides the compound of Formula (IV), wherein X is C; and R₄ is C₁-C₇ straight or branched alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A further embodiment provides a compound of Formula (V):

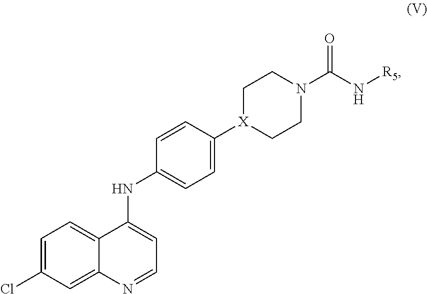

wherein: X is selected from the group of N and C; and R₅ is selected from the group of H, C₁-C₇ straight or branched alkyl, and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A different embodiment provides a compound of Formula (V), wherein: X is selected from the group of N and C; and R₅ is selected from the group of C₁-C₇ straight or branched alkyl and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still different embodiment provides a compound of Formula (V), wherein: X is N; and R₅ is selected from the group of C₁-C₇ straight or branched alkyl and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still different embodiment provides a compound of Formula (V), wherein: X is C; and R₅ is selected from the group of C₁-C₇ straight or branched alkyl and C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still different embodiment provides a compound of Formula (V), wherein: X is N; and R₅ is C₁-C₇ straight or branched alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still different embodiment provides a compound of Formula (V), wherein: X is N; and R₅ is C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still different embodiment provides a compound of Formula (V), wherein: X is C; and R₅ is C₁-C₇ straight or branched alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still different embodiment provides a compound of Formula (V), wherein: X is C; and R₅ is C₃-C₁₀ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof. Also provided is a method for inhibiting malaria in a subject, the method comprising administering to the subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof. In some embodiments, the subject in the methods described herein is a human subject.

The compounds and pharmaceutical compositions described herein may be used to treat malaria in a subject. Species of parasites that may be targeted with antimalarial treatment include all species capable of causing human or animal infection, including Plasmodium falciparum, Plasmodium vivax, Plasmodium ovale, Plasmodium knowlesi, and Plasmodium malariae.

In some embodiments, the infecting agent is resistant to one antimalarial agent, which may be referred to as drug resistant malaria.

A multi-drug resistant malaria refers to a malarial infection that has proven to be resistant to treatment with two or more known agents for the treatment of malaria, or caused by an infective species known to be resistant to treatment with two or more of such, such as a multi-drug resistant Plasmodium species. In some embodiments, the multi-drug resistant species is a Plasmodium falciparum species.

In some embodiments, the infecting species in question, such as a Plasmodium species, is resistant to one or more anti-malarial agents or combinations thereof, including those selected from the group of chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, artemisinin derivatives (including dihydroaremisinin (DHA), artesunate, artmether, arteether), artemisinin-based combination therapies (ACT), such as DHA-piperaquine and DHA-piperaquine mefloquine-artesunate. It is understood that reference to one or more of these antimalarial agents includes pharmaceutically acceptable salts thereof.

Also provided is a method of treating coccidiosis in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the subject to be treated for coccidiosis is a human subject. In other embodiments, the subject is a veterinary subject. In some embodiments, the veterinary subject is poultry, such as a chicken. In other embodiments, the veterinary subject is a mammal subject, such as selected from the group of cattle, horses, dogs, cats, sheep, goats, pigs, and rabbits.

Another embodiment provides a method of treating coccidiosis in a poultry subject, the method comprising administering to a subject in need thereof: a) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and b) a pharmaceutically effective amount of decoquinate, or a pharmaceutically acceptable salt thereof.

In some embodiments, the compound of Formula (I) is a administered to the poultry, such as chickens, in their feed at a concentration of from about 1 mg/kg to about 100 mg/kg.

In embodiments herein for treating veterinary subjects, it is understood that the active compound of Formula (I) or other pharmaceutical agents may be incorporated into animal feed at a desired concentration using techniques known in the art, including micronization or nanosization of the material, including the mechanical methods of milling, grinding, and cutting, as well as the use of supercritical fluids in supersaturation and precipitation techniques, such as Rapid Expansion of Supercritical Solutions (RES S), the Supercritical Anti-Solvent method (SAS), and Particles from Gas Saturated Solutions method (PGSS).

Also provided is a method of treating babesiosis (also known as a piroplasmosis) in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the subject of the piroplasmosis is infected by a Theileria species, such as T. equi (horses, donkeys, mules, and dogs), T. annae (dogs).

In other embodiments, the subject of the piroplasmosis is infected by a Babesia species, such as B. microti (human), B. duncani (human), Babesia canis (dogs), B. gibsoni (dogs), Babesia fells (cats), Babesia caballi (horses, donkeys, mules), Babesia ovis (sheep), Babesia motasi (sheep), Babesia odocoilei (deer and reindeer), B. orientalis (Buffalo).

In cattle, the Babesia infection in the subject may be from a species selected from the group of B. divergens, Babesia bigemina, Babesia bovis, B. beliceri, B. jakimovi, B. major, B. occultans, and B. ovata. In pigs, the Babesia infection in the subject may be from a species selected from the group of B. perroncitoi and B. trautmanni.

In small ruminants, such as sheep and goats, the Babesia infection in the subject may be from a species selected from the group of Babesia motasi, B. ovis, and B. crassa.

### A. Definitions

The term “therapeutically effective amount” or “pharmaceutically effective amount” refers to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula (I), or a pharmaceutically acceptable salt or co-crystal thereof, is an amount sufficient to modulate a malarial infection (or other Apicomplexa infection described herein), and thereby treat a subject (e.g., a human) suffering from the infection, or to ameliorate or alleviate the existing symptoms of the infection. For example, a therapeutically or pharmaceutically effective amount may be an amount sufficient to decrease a symptom of a malarial infection (or other Apicomplexa infection described herein), as described herein.

In some embodiments, for human administration, each dosage unit contains from 0.1 mg to 1 g, 0.1 mg to 700 mg, or 0.1 mg to 100 mg of a compound of Formula (I), or a pharmaceutically acceptable salt or co-crystal thereof. In some embodiments, a therapeutically effective amount or a pharmaceutically effective amount of a compound of Formula (I), or a pharmaceutically acceptable salt thereof, comprises from about 0.1 mg to about 500 mg per dose, given once or twice daily. In some embodiments, the individual dose is selected from 1 mg, 5 mg, 10 mg, 20 mg, 30 mg, 40 mg, 50 mg, 60 mg, 75 mg, 100 mg, 150 mg, 200 mg, 250 mg, 300 mg, 350 mg, 400 mg, 500 mg, 600 mg, 700 mg, 750 mg, 800 mg, 900 mg, and 1 g per dose.

The terms “subject,” “patient,” or “recipient” refer to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula (I), or a pharmaceutically acceptable salt or co-crystal thereof, as described herein. This includes a subject who may be determined to be at risk of or susceptible to such diseases or conditions, such that treatment would prevent the disease or condition from developing.

The term “alkyl” refers to a straight or branched hydrocarbon. For example, an alkyl group can have 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, CH₂CH₂CH₃), 2-propyl (i-Pr, i-propyl, —CH(CH₃)₂), 1-butyl (n-Bu, n-butyl, CH₂CH₂CH₂CH₃), 2-methyl-1-propyl (i-Bu, i-butyl, —CH₂CH(CH₃)₂), 2-butyl (s-Bu, s-butyl, CH(CH₃)CH₂CH₃), 2-methyl-2-propyl (t-Bu, t-butyl, —C(CH₃)₃), 1-pentyl (n-pentyl, CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃) 2 CH₂CH₃), 3-methyl-2-butyl (—CH(CH₃)CH(CH₃) 2), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃) 3.

The term “cycloalkyl” refers to a saturated ring having from 3 to 10 carbon atoms (C₃-C₁₀ cycloalkyl) as a monocycle, including cyclopropyl, cyclobutyl, cyclopentyl, cyclohexyl, cycloheptyl, and cyclooctyl groups, as well as bicyclic, bridged, and spirocyclic hydrocarbon rings, such as spiro[4.4]nonanyl, bicyclo[2.1.1]hexanyl, bicyclo[1.1.1]pentanyl, decahydronaphthalenyl (decalinyl), bicyclo[2.2.1]heptanyl, bicyclo[3.1.1]heptanyl, bicyclo[2.2.2]octanyl, and adamantyl groups.

The term “halomethyl” refers to a methyl group in which one or more hydrogen atoms of the methyl group is replaced with a halogen atom. Examples include the fluoromethyl groups —CH₂F, —CHF₂, and —CF₃. Additional halomethyl groups include those wherein the halogen substitution is with one, two, or three halogen atoms selected from the group of bromine, iodine, or chlorine atoms are also understood for use herein.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula (I), and a guest (or co-former) molecule or molecules. In particular embodiments the pharmaceutically acceptable co-crystal of the compound of Formula (I) with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

The term “co-crystal” also means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

The term “pharmaceutically acceptable salt” or “therapeutically acceptable salt” refer to a salt form of a compound of Formula (I) which is, within the scope of sound medical evaluation, suitable for use in contact with the tissues and organs of humans and/or animals such that any resulting toxicity, irritation, allergic response, and the like and are commensurate with a reasonable benefit/risk ratio.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The terms “carrier” or “pharmaceutically acceptable carrier” refer to an excipient or vehicle that includes without limitation diluents, disintegrants, precipitation inhibitors, surfactants, glidants, binders, lubricants, and the like with which the compound is administered. Carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

All ranges disclosed and/or claimed herein are inclusive of the recited endpoint and independently combinable. For example, the ranges of “from 2 to 10,” “2 through 10,” and “2-10” are inclusive of the endpoints, 2 and 10, and all the intermediate values between in context of the units considered. For instance, reference to “Claims 2-10,” “C₂-C₁₀ alkyl,” and C₂₋₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

### B. Compounds

In one aspect, the invention relates to substituted N-(4-(piperidin-4-yl)phenyl)quinolin-4-amines and substituted N-(4-(piperazin-1-yl)phenyl)quinolin-4-amines useful in, for example, the treatment of malaria (e.g., drug-resistant malaria, multidrug-resistant malaria). Thus, in one aspect, the compounds of the invention are useful in the treatment of malaria as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds of Formula (I), or a pharmaceutically acceptable salt thereof:

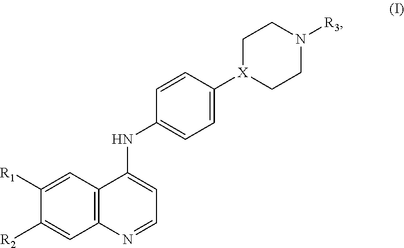

wherein: R₁ is selected from H and halogen; R₂ is selected from H, halogen, and halomethyl; R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and X is selected from N and C; with the proviso that, when R₁ is H, R₂ is not H; with the proviso that, when R₂ is H, R₁ is not H; and with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof.

In one aspect, disclosed are compounds selected from:

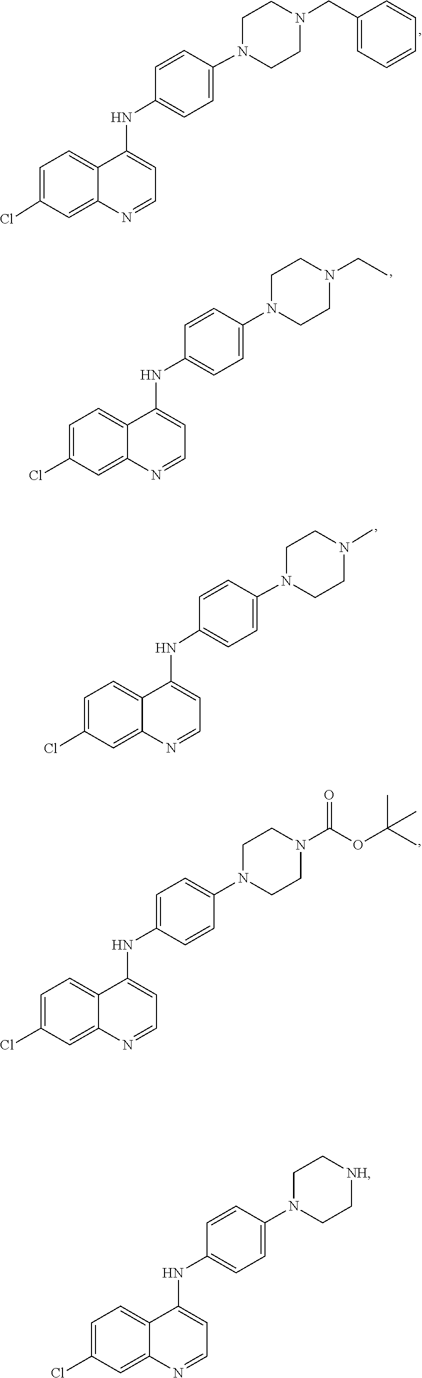 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1]

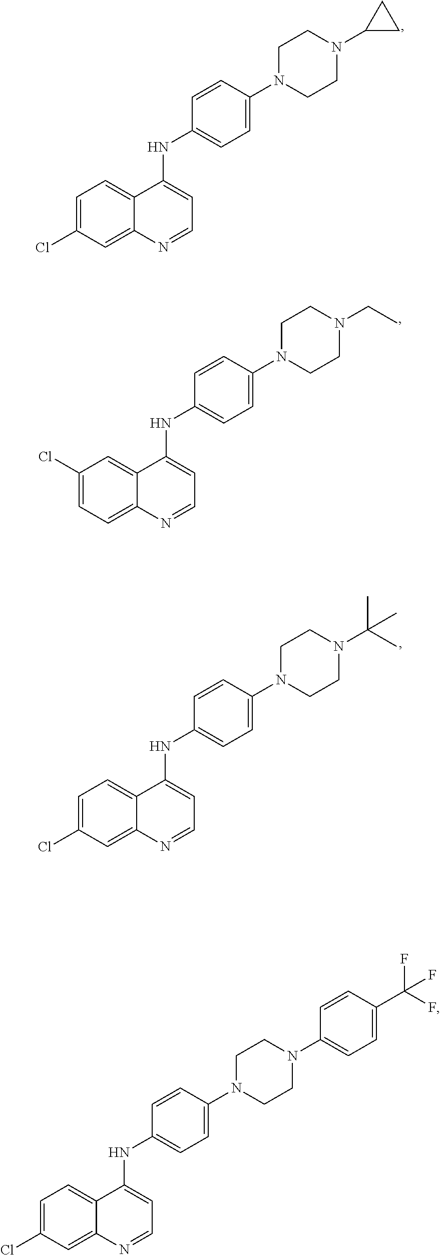 [SMILES: Clc1ccc2c(Nc3ccc(N4CCN(C5CC5)CC4)cc3)ccnc2c1 ; CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1]

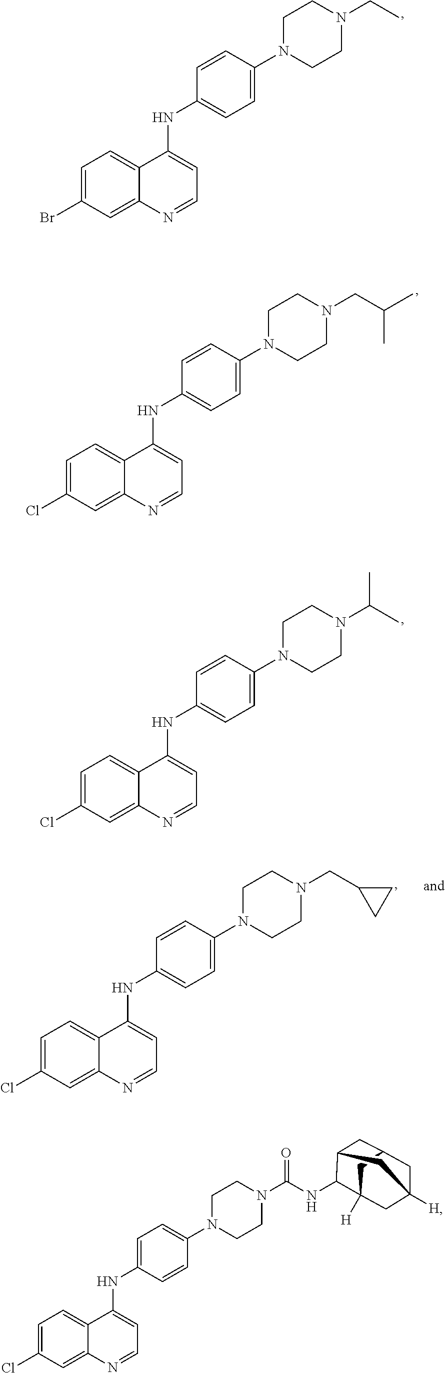

or a pharmaceutically acceptable salt thereof.

In various aspects, the compound, or a pharmaceutically acceptable salt thereof, has a Formula (I):

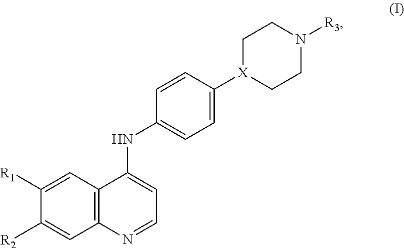

wherein: R₁ is selected from H and halogen; R₂ is selected from H, halogen, and halomethyl; R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and X is selected from N and C; with the proviso that one or more selected from: (a) X is C and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; (b) R₁ is halogen and R₂ is selected from H and halomethyl; and (c) R₂ is halomethyl and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl.

In various aspects, the compound, or a pharmaceutically acceptable salt thereof, has a Formula (VI):

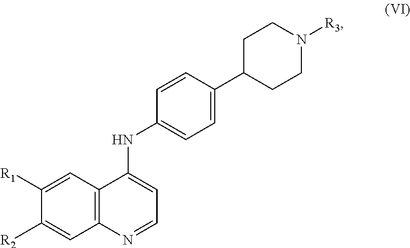

wherein: R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl. In a further aspect, R₁ is halogen and R₂ is selected from H and halomethyl; or a pharmaceutically acceptable salt thereof. In a still further aspect, R₂ is halomethyl and R₃ is selected from H, —C(═O)—Ci-C, straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; or a pharmaceutically acceptable salt thereof.

In various aspects, R₁ is selected from H, and C₁; R₂ is selected from H, Cl, and fluoromethyl; and R₃ is selected from H, C₁-C₇ straight or branched alkyl, C₃-C₆ cycloalkyl, —CH₂—C₃-C₆ cycloalkyl, and benzyl; X is selected from N and C; with the proviso that, when R₁ is H, R₂ is not H; and with the proviso that, when R₂ is H, R₁ is not H; and with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof.

In various aspects, R₁ is H; R₂ is selected from C₁ and fluoromethyl; and R₃ is selected from H, C₁-C₇ straight or branched alkyl, C₃-C₆ cycloalkyl, —CH₂—C₃-C₆ cycloalkyl, and benzyl; X is selected from N and C; with the proviso that, when R₁ is H, R₂ is not H; with the proviso that, when R₂ is H, R₁ is not H; and with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof.

In various aspects, R₁ is H; R₂ is selected from C₁ and CF₃; and R₃ is selected from H, C₁-C₇ straight or branched alkyl, C₃-C₆ cycloalkyl, —CH₂—C₃-C₆ cycloalkyl, and benzyl; and X is selected from N and C; with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; or 7-chloro-N-(4-(1-methylpiperidin-4-yl)phenyl)quinolin-4-amine; or a pharmaceutically acceptable salt thereof.

In various aspects, the compound or a pharmaceutically acceptable salt thereof, has Formula (II):

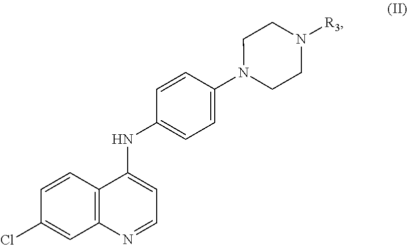

wherein R₃ is selected from H, C₁-C₇ straight or branched alkyl, C₃-C₆ cycloalkyl, —CH₂—C₃-C₆ cycloalkyl, and benzyl; with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; or 7-chloro-N-(4-(1-methylpiperidin-4-yl)phenyl)quinolin-4-amine; or a pharmaceutically acceptable salt thereof. In a further aspect, R₃ is selected from H, ethyl, n-propyl, isopropyl, tert-butyl, isobutyl, sec-butyl, n-pentyl, pentan-2-yl, 2-methylbutyl, isopentyl, 3-methylbutan-2-yl, n-hexyl, isohexyl, t-hexyl, sec-hexyl, 2-methylpentyl, 3-methylpentyl, n-heptyl, 5-methylhexyl, t-heptyl, sec-heptyl, and iso-heptyl; or a pharmaceutically acceptable salt thereof. In a still further aspect, R₃ is selected from H, ethyl, n-propyl, isopropyl, tert-butyl, isobutyl, and sec-butyl; or a pharmaceutically acceptable salt thereof. In yet a further aspect, R₃ is selected from H, C₁-C₄ straight or branched alkyl, C₃-C₆ cycloalkyl, —CH₂—C₃-C₆ cycloalkyl, and benzyl; or a pharmaceutically acceptable salt thereof; with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; or 7-chloro-N-(4-(1-methylpiperidin-4-yl)phenyl)quinolin-4-amine; or a pharmaceutically acceptable salt thereof. In an even further aspect, R₃ is selected from H, C₁-C₄ straight or branched alkyl, C₃-C₆ cycloalkyl, and benzyl; or a pharmaceutically acceptable salt thereof; with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; or 7-chloro-N-(4-(1-methylpiperidin-4-yl)phenyl)quinolin-4-amine; or a pharmaceutically acceptable salt thereof. In a still further aspect, R₃ is C₁-C₄ straight or branched alkyl; or a pharmaceutically acceptable salt thereof; with the proviso that the compound is not N-(4-(4-butylpiperazin-1-yl)phenyl)-7-chloroquinolin-4-amine; or 7-chloro-N-(4-(4-methylpiperazin-1-yl)phenyl)quinolin-4-amine; or a pharmaceutically acceptable salt thereof.

In various aspects, the compound, or a pharmaceutically acceptable salt thereof, has Formula (III):

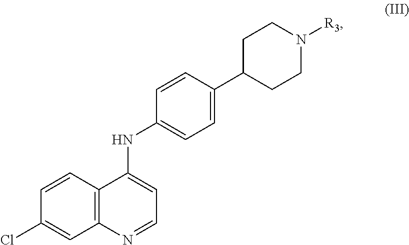

wherein R₃ is selected from H and C₂-C₇ straight or branched alkyl; or a pharmaceutically acceptable salt thereof.

In various aspects, the compound is selected from:

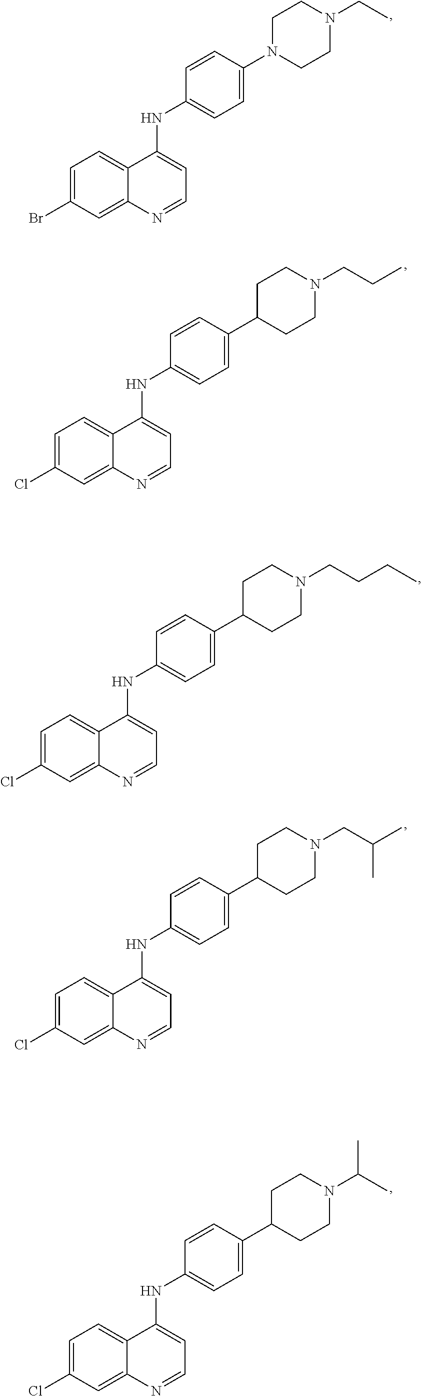 [SMILES: CCN1CCN(c2ccc(Nc3ccnc4cc(Br)ccc34)cc2)CC1 ; CCCN1CCC(c2ccc(Nc3ccnc4cc(Cl)ccc34)cc2)CC1 ; CCCCN1CCC(c2ccc(Nc3ccnc4cc(Cl)ccc34)cc2)CC1 ; CC(C)CN1CCC(c2ccc(Nc3ccnc4cc(Cl)ccc34)cc2)CC1 ; CC(C)N1CCC(c2ccc(Nc3ccnc4cc(Cl)ccc34)cc2)CC1]

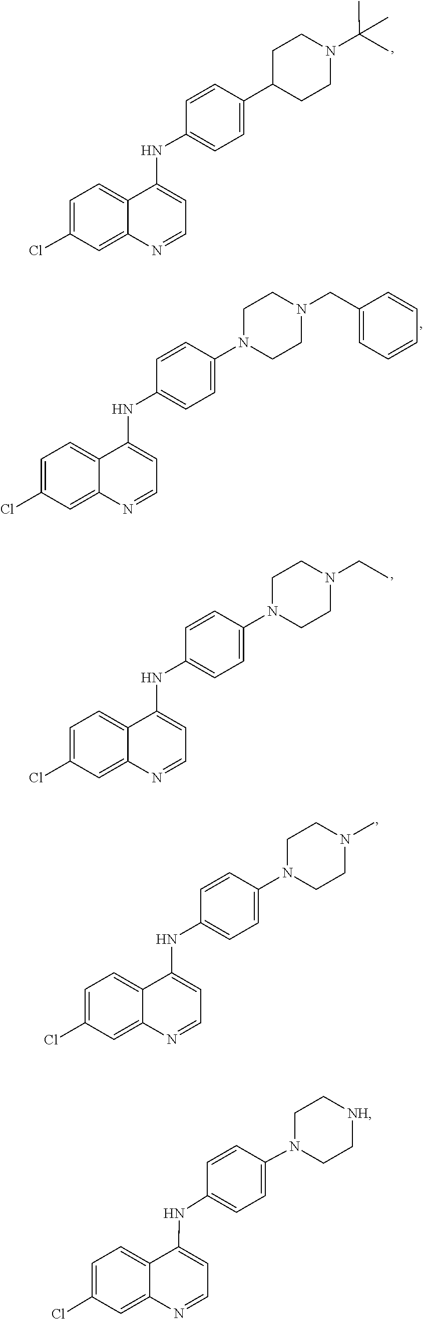 [SMILES: CC(C)(C)N1CCC(c2ccc(Nc3ccnc4cc(Cl)ccc34)cc2)CC1 ; Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; Clc1ccc2c(Nc3ccc(N4CCNCC4)cc3)ccnc2c1]

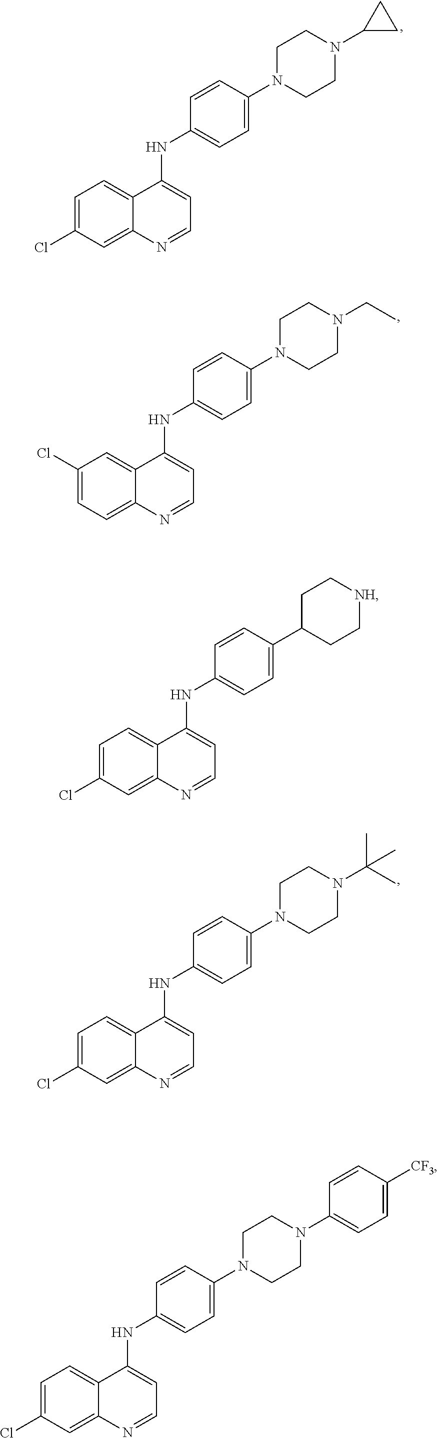 [SMILES: Clc1ccc2c(Nc3ccc(N4CCN(C5CC5)CC4)cc3)ccnc2c1 ; CCN1CCN(c2ccc(Nc3ccnc4ccc(Cl)cc34)cc2)CC1 ; Clc1ccc2c(Nc3ccc(C4CCNCC4)cc3)ccnc2c1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1]

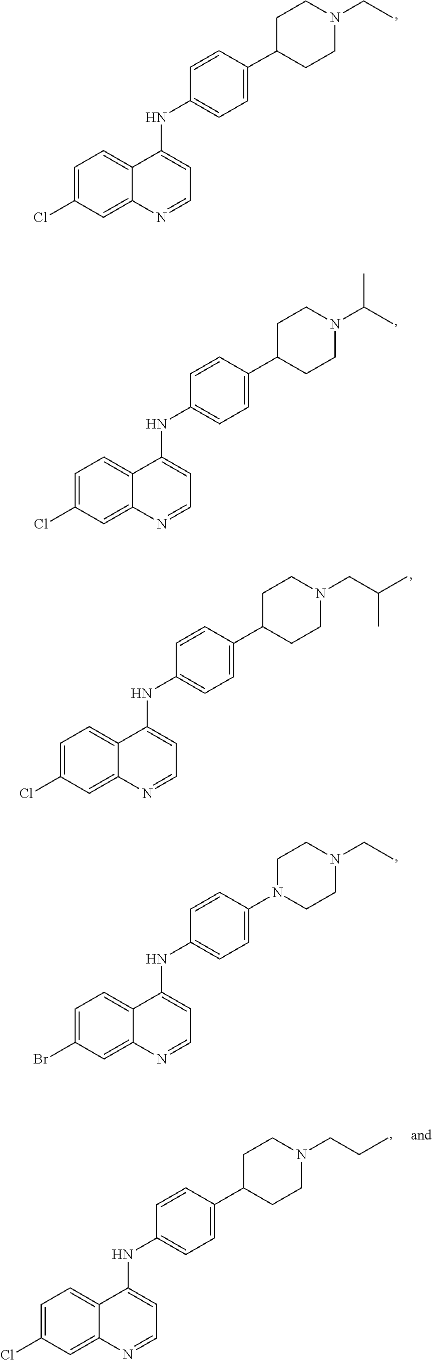 [SMILES: CCN1CCC(c2ccc(Nc3ccnc4cc(Cl)ccc34)cc2)CC1 ; CC(C)N1CCC(c2ccc(Nc3ccnc4cc(Cl)ccc34)cc2)CC1 ; CC(C)CN1CCC(c2ccc(Nc3ccnc4cc(Cl)ccc34)cc2)CC1 ; CCN1CCN(c2ccc(Nc3ccnc4cc(Br)ccc34)cc2)CC1 ; CCCN1CCC(c2ccc(Nc3ccnc4cc(Cl)ccc34)cc2)CC1]

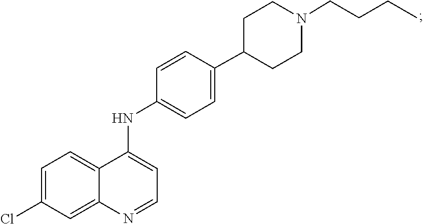 [SMILES: CCCCN1CCC(c2ccc(Nc3ccnc4cc(Cl)ccc34)cc2)CC1]

or a pharmaceutically acceptable salt thereof.

In various aspects, the compound has Formula (IV):

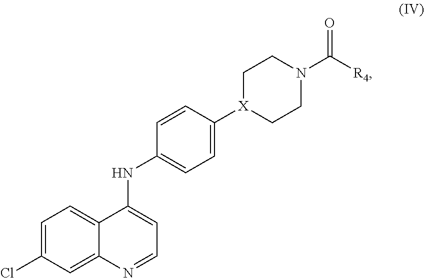

wherein: X is selected group of N and C; and R₄ is selected from H, C₁-C₇ straight or branched alkyl, and C₃-C₁₀ cycloalkyl; or a pharmaceutically acceptable salt thereof. In a further aspect, X is N; and R₄ is selected from H, C₁-C₇ straight or branched alkyl, and C₃-C₁₀ cycloalkyl; or a pharmaceutically acceptable salt thereof. In a still further aspect, X is C; and R₄ is selected from H, C₁-C₇ straight or branched alkyl, and C₃-C₁₀ cycloalkyl; or a pharmaceutically acceptable salt thereof. In yet a further aspect, X is N; and R₄ is C₁-C₇ straight or branched alkyl; or a pharmaceutically acceptable salt thereof. In an even further aspect, X is C; and R₄ is C₁-C₇ straight or branched alkyl; or a pharmaceutically acceptable salt thereof.

In various aspects, the compound has Formula (V):

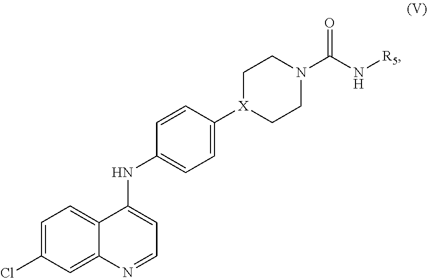

wherein: X is selected from N and C; and R₅ is selected from H, C₁-C₇ straight or branched alkyl, and C₃-C₁₀ cycloalkyl; or a pharmaceutically acceptable salt thereof. In a further aspect, X is selected from N and C; and R₅ is selected from C₁-C₇ straight or branched alkyl and C₃-C₁₀ cycloalkyl; or a pharmaceutically acceptable salt thereof. In a still further aspect, X is N; and R₅ is selected from C₁-C₇ straight or branched alkyl and C₃-C₁₀ cycloalkyl; or a pharmaceutically acceptable salt thereof. In yet a further aspect, X is C; and R₅ is selected from C₁-C₇ straight or branched alkyl and C₃-C₁₀ cycloalkyl; or a pharmaceutically acceptable salt thereof. In an even further aspect, X is N; and R₅ is C₁-C₇ straight or branched alkyl; or a pharmaceutically acceptable salt thereof. In a still further aspect, X is N; and R₅ is C₃-C₁₀ cycloalkyl; or a pharmaceutically acceptable salt thereof. In yet a further aspect, X is C; and R₅ is C₁-C₇ straight or branched alkyl; or a pharmaceutically acceptable salt thereof. In an even further aspect, X is C; and R₅ is C₃-C₁₀ cycloalkyl; or a pharmaceutically acceptable salt thereof.

In various aspects, the compound is selected from:

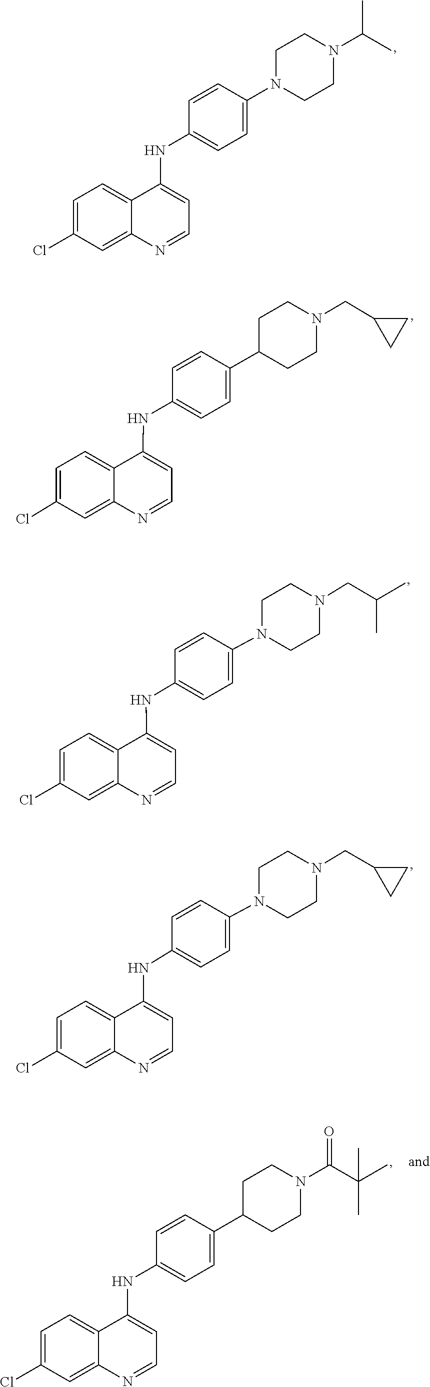 [SMILES: CC(C)N1CCN(c2ccc(Nc3ccnc4cc(Cl)ccc34)cc2)CC1 ; Clc1ccc2c(Nc3ccc(C4CCN(CC5CC5)CC4)cc3)ccnc2c1 ; CC(C)CN1CCN(c2ccc(Nc3ccnc4cc(Cl)ccc34)cc2)CC1 ; Clc1ccc2c(Nc3ccc(N4CCN(CC5CC5)CC4)cc3)ccnc2c1 ; CC(C)(C)C(=O)N1CCC(c2ccc(Nc3ccnc4cc(Cl)ccc34)cc2)CC1]

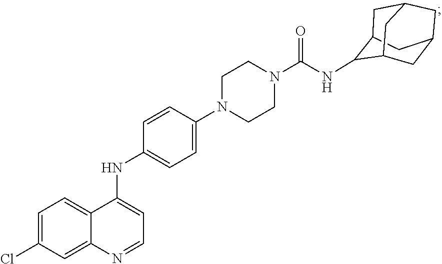 [SMILES: O=C(NC1C2CC3CC(C2)CC1C3)N1CCN(c2ccc(Nc3ccnc4cc(Cl)ccc34)cc2)CC1]

or a pharmaceutically acceptable salt thereof.

In various aspects, the compound is selected from:

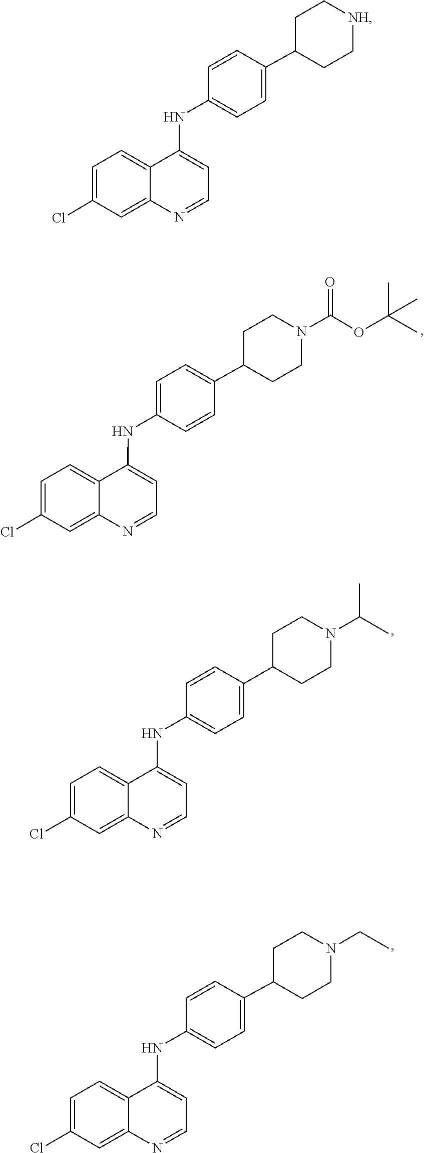 [SMILES: Clc1ccc2c(Nc3ccc(C4CCNCC4)cc3)ccnc2c1 ; CC(C)(C)OC(=O)N1CCC(c2ccc(Nc3ccnc4cc(Cl)ccc34)cc2)CC1 ; CC(C)N1CCC(c2ccc(Nc3ccnc4cc(Cl)ccc34)cc2)CC1 ; CCN1CCC(c2ccc(Nc3ccnc4cc(Cl)ccc34)cc2)CC1]

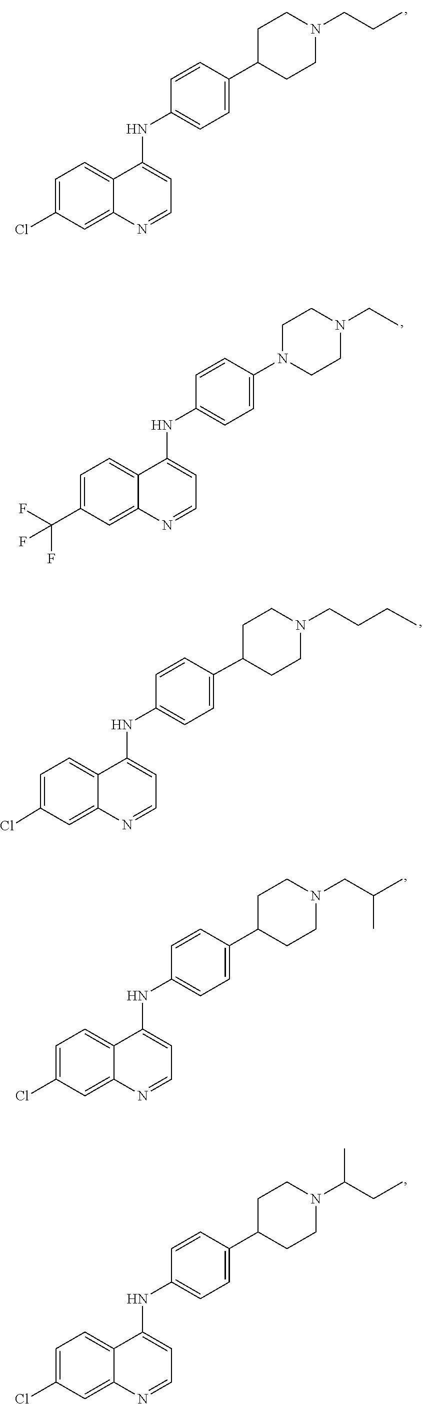 [SMILES: CCCN1CCC(c2ccc(Nc3ccnc4cc(Cl)ccc34)cc2)CC1 ; CCN1CCN(c2ccc(Nc3ccnc4cc(C(F)(F)F)ccc34)cc2)CC1 ; CCCCN1CCC(c2ccc(Nc3ccnc4cc(Cl)ccc34)cc2)CC1 ; CC(C)CN1CCC(c2ccc(Nc3ccnc4cc(Cl)ccc34)cc2)CC1 ; CCC(C)N1CCC(c2ccc(Nc3ccnc4cc(Cl)ccc34)cc2)CC1]

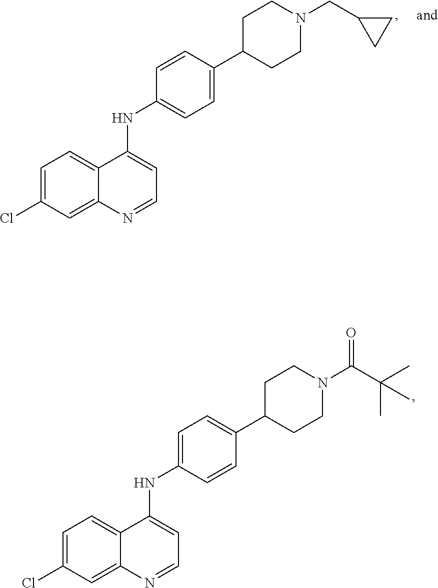 [SMILES: Clc1ccc2c(Nc3ccc(C4CCN(CC5CC5)CC4)cc3)ccnc2c1 ; CC(C)(C)C(=O)N1CCC(c2ccc(Nc3ccnc4cc(Cl)ccc34)cc2)CC1]

or a pharmaceutically acceptable salt thereof.

In various aspects, the compound is selected from:

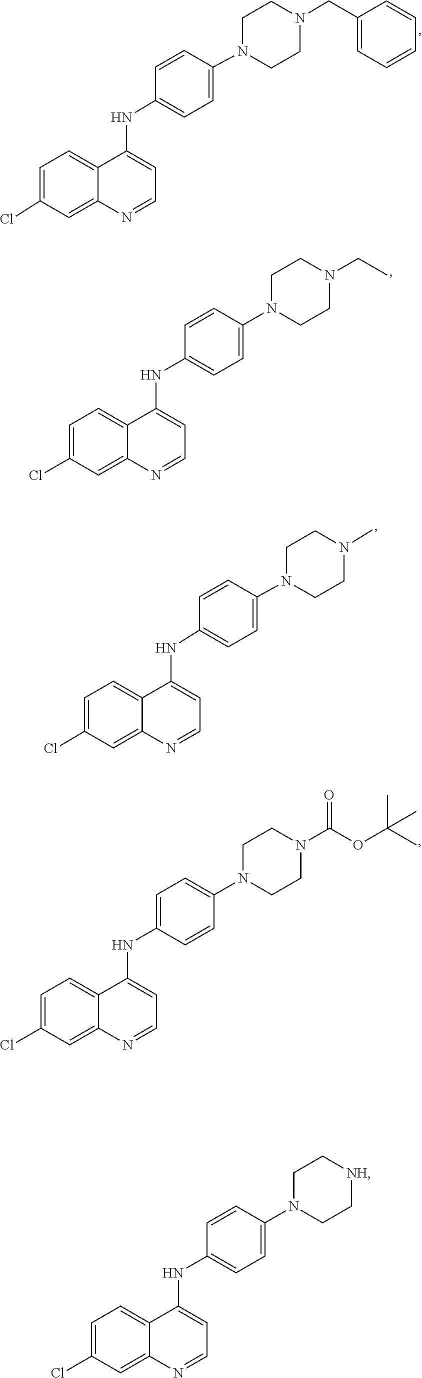 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1]

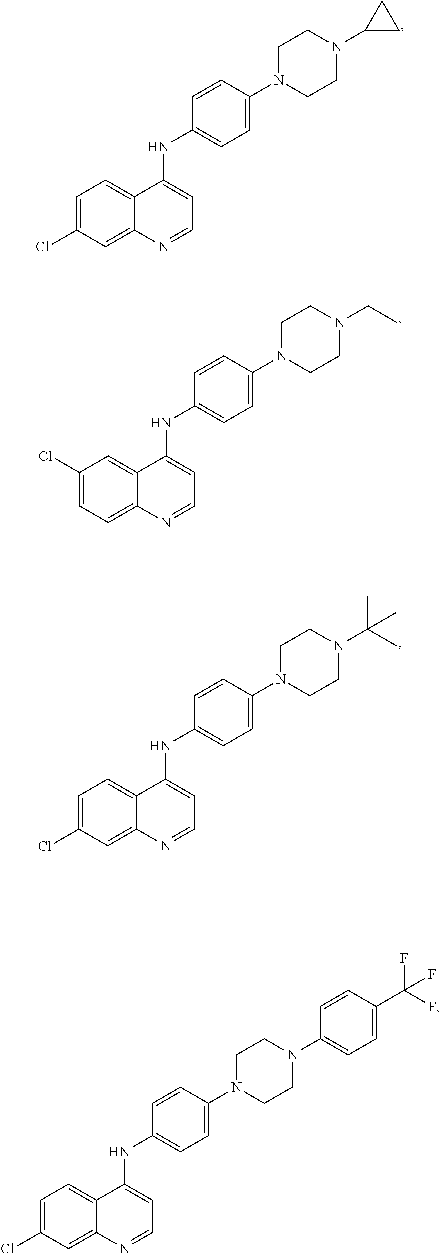 [SMILES: Clc1ccc2c(Nc3ccc(N4CCN(C5CC5)CC4)cc3)ccnc2c1 ; CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1]

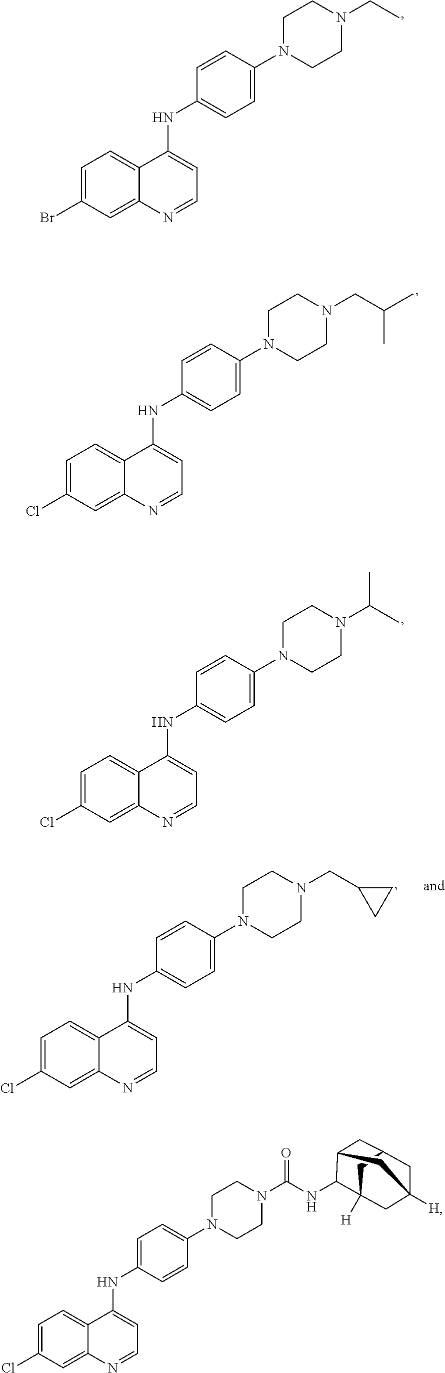

or a pharmaceutically acceptable salt thereof.

a. X Groups

In one aspect, X is selected from N and C. In a further aspect, X is N. In a still further aspect, X is C.

b. R₁ Groups

In one aspect, R₁ is selected from H and halogen. In a further aspect, R₁ is selected from H, —F, —Cl, and —Br. In a still further aspect, R₁ is selected from H, —F, and —Cl. In yet a further aspect, R₁ is selected from H and —Cl. In a still further aspect, R₁ is selected from H and —F.

In various aspects, R₁ is halogen. In a further aspect, R₁ is selected from —F, —Cl, and —Br. In a still further aspect, R₁ is selected from —F, and —Cl. In yet a further aspect, R₁ is —Cl. In a still further aspect, R₁ is —F.

In various aspects, R₁ is H.

C. R₂ Groups

In one aspect, R₂ is selected from H, halogen, and halomethyl. In a further aspect, R₂ is selected from H, —F, —Cl, —Br, —CH₂F, —CH₂Cl, —CHF₂, —CHCl₂, —CF₃, —CCl₃, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In a still further aspect, R₂ is selected from H, —F, —Cl, —CH₂F, —CH₂Cl, —CHF₂, —CHCl₂, —CF₃, —CCl₃, —CH₂CH₂F, and —CH₂CH₂Cl. In yet a further aspect, R₂ is selected from H, —F, —Cl, —CH₂F, —CH₂Cl, —CHF₂, —CHCl₂, —CF₃, and —CCl₃.

In various aspects, R₂ is selected from halogen and halomethyl. In a further aspect, R₂ is selected from —F, —Cl, —Br, —CH₂F, —CH₂Cl, —CHF₂, —CHCl₂, —CF₃, —CCl₃, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In a still further aspect, R₂ is selected from —F, —Cl, —CH₂F, —CH₂Cl, —CHF₂, —CHCl₂, —CF₃, —CCl₃, —CH₂CH₂F, and —CH₂CH₂Cl. In yet a further aspect, R₂ is selected from —F, —Cl, —CH₂F, —CH₂Cl, —CHF₂, —CHCl₂, —CF 3, and —CCl₃.

In various aspects, R₂ is halomethyl. In a further aspect, R₂ is selected from —CH₂F, —CH₂Cl, —CHF 2, —CHCl₂, —CF₃, —CCl₃, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In a still further aspect, R₂ is selected from —CH₂F, —CH₂Cl, —CHF₂, —CHCl₂, —CF₃, —CCl₃, —CH₂CH₂F, and —CH₂CH₂Cl. In yet a further aspect, R₂ is selected from —CH₂F, —CH₂Cl, —CHF₂, —CHCl₂, —CF₃, and —CCl₃.

In various aspects, R₂ is halogen. In a further aspect, R₂ is selected from —F, —Cl, and —Br. In a still further aspect, R₂ is selected from —F and —Cl. In yet a further aspect, R₂ is —Cl. In an even further aspect, R₂ is —F.

In various aspects, R₂ is H.

d. R₃ Groups

In one aspect, R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl. In a further aspect, R₃ is selected from H, C₁-C₄ straight or branched alkyl, —C(═O)—C₁-C₄ straight or branched alkyl, —C(═O)—NH—C₁-C₄ straight or branched alkyl, C₃-C₆ cycloalkyl, —CH₂—C₃-C₆ cycloalkyl, —C(═O)—C₃-C₆ cycloalkyl, —C(═O)—NH—C₃-C₆ cycloalkyl, and benzyl. In a still further aspect, R₃ is selected from H, methyl, ethyl, n-propyl, isopropyl, —C(═O)CH₃, —C(═O)CH₂CH₃, —C(═O)CH₂CH₂CH₃, —C(═O)CH(CH₃)₂, —C(═O)NHCH₃, —C(═O)NHCH₂CH₃, —C(═O)NHCH₂CH₂CH₃, —C(═O)NHCH(CH₃)₂, cyclopropyl, cyclobutyl, cyclopentyl, —CH₂-cyclopropyl, —CH₂-cyclobutyl, —CH₂-cyclopentyl, —C(═O)-cyclopropyl, —C(═O)-cyclobutyl, —C(═O)-cyclopentyl, —C(═O)NH-cyclopropyl, —C(═O)NH-cyclobutyl, —C(═O)NH-cyclopentyl, and benzyl. In yet a further aspect, R₃ is selected from H, methyl, ethyl, —C(═O)CH₃, —C(═O)CH₂CH₃, —C(═O)NHCH₃, —C(═O)NHCH₂CH₃, cyclopropyl, cyclobutyl, —CH₂-cyclopropyl, —CH₂-cyclobutyl, —C(═O)-cyclopropyl, —C(═O)-cyclobutyl, —C(═O)NH-cyclopropyl, —C(═O)NH-cyclobutyl, and benzyl. In an even further aspect, R₃ is selected from H, methyl, —C(═O)CH₃, —C(═O)NHCH₃, cyclopropyl, —CH₂-cyclopropyl, —C(═O)-cyclopropyl, —C(═O)NH-cyclopropyl, and benzyl.

In various aspects, R₃ is selected from H, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl. In a further aspect, R₃ is selected from H, —C(═O)—C₁-C₄ straight or branched alkyl, —C(═O)—NH—C₁-C₄ straight or branched alkyl, C₃-C₆ cycloalkyl, —CH₂—C₃-C₆ cycloalkyl, —C(═O)—C₃-C₆ cycloalkyl, —C(═O)—NH—C₃-C₆ cycloalkyl, and benzyl. In a still further aspect, R₃ is selected from H, —C(═O)CH₃, —C(═O)CH₂CH₃, —C(═O)CH₂CH₂CH₃, —C(═O)CH(CH₃)₂, —C(═O)NHCH₃, —C(═O)NHCH₂CH₃, —C(═O)NHCH₂CH₂CH₃, —C(═O)NHCH(CH₃)₂, cyclopropyl, cyclobutyl, cyclopentyl, —CH₂-cyclopropyl, —CH₂-cyclobutyl, —CH₂-cyclopentyl, —C(═O)-cyclopropyl, —C(═O)-cyclobutyl, —C(═O)-cyclopentyl, —C(═O)NH-cyclopropyl, —C(═O)NH-cyclobutyl, —C(═O)NH-cyclopentyl, and benzyl. In yet a further aspect, R₃ is selected from H, —C(═O)CH₃, —C(═O)CH₂CH₃, —C(═O)NHCH₃, —C(═O)NHCH₂CH₃, cyclopropyl, cyclobutyl, —CH₂-cyclopropyl, —CH₂-cyclobutyl, —C(═O)-cyclopropyl, —C(═O)-cyclobutyl, —C(═O)NH-cyclopropyl, —C(═O)NH-cyclobutyl, and benzyl. In an even further aspect, R₃ is selected from H, —C(═O)CH₃, —C(═O)NHCH₃, cyclopropyl, —CH₂-cyclopropyl, —C(═O)-cyclopropyl, —C(═O)NH-cyclopropyl, and benzyl.

In various aspects, R₃ is —C(═O)—C₁-C₇ straight or branched alkyl. In a further aspect, R₃ is —C(═O)—C₁-C₄ straight or branched alkyl. In a still further aspect, R₃ is selected from —C(═O)CH₃, —C(═O)CH₂CH₃, —C(═O)CH₂CH₂CH₃, and —C(═O)CH(CH₃)₂. In yet a further aspect, R₃ is selected from —C(═O)CH₃ and —C(═O)CH₂CH₃. In an even further aspect, R₃ is —C(═O)CH₃.

In various aspects, R₃ is selected from C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl. In a further aspect, R₃ is selected from C₃-C₆ cycloalkyl, —CH₂—C₃-C₆ cycloalkyl, —C(═O)—C₃-C₆ cycloalkyl, and —C(═O)—NH—C₃-C₆ cycloalkyl. In a still further aspect, R₃ is selected from cyclopropyl, cyclobutyl, cyclopentyl, —CH₂-cyclopropyl, —CH₂-cyclobutyl, —CH₂-cyclopentyl, —C(═O)-cyclopropyl, —C(═O)-cyclobutyl, —C(═O)-cyclopentyl, —C(═O)NH-cyclopropyl, —C(═O)NH-cyclobutyl, and —C(═O)NH-cyclopentyl. In yet a further aspect, R₃ is selected from cyclopropyl, cyclobutyl, —CH₂-cyclopropyl, —CH₂-cyclobutyl, —C(═O)-cyclopropyl, —C(═O)-cyclobutyl, —C(═O)NH-cyclopropyl, and —C(═O)NH-cyclobutyl. In an even further aspect, R₃ is selected from cyclopropyl, —CH₂-cyclopropyl, —C(═O)-cyclopropyl, and —C(═O)NH-cyclopropyl.

In various aspects, R₃ is selected from H, C₁-C₇ straight or branched alkyl, C₃-C₆ cycloalkyl, —CH₂—C₃-C₆ cycloalkyl, and benzyl. In a further aspect, R₃ is selected from H, C₁-C₄ straight or branched alkyl, C₃-C₆ cycloalkyl, —CH₂—C₃-C₆ cycloalkyl, and benzyl. In a still further aspect, R₃ is selected from H, methyl, ethyl, n-propyl, isopropyl, cyclopropyl, cyclobutyl, cyclopentyl, —CH₂-cyclopropyl, —CH₂-cyclobutyl, —CH₂-cyclopentyl, and benzyl. In yet a further aspect, R₃ is selected from H, methyl, ethyl, cyclopropyl, cyclobutyl, —CH₂-cyclopropyl, —CH₂-cyclobutyl, and benzyl. In an even further aspect, R₃ is selected from H, methyl, cyclopropyl, —CH₂-cyclopropyl, and benzyl.

In various aspects, R₃ is selected from H, C₁-C₄ straight or branched alkyl, C₃-C₆ cycloalkyl, and benzyl. In a further aspect, R₃ is selected from H, C₁-C₄ straight or branched alkyl, C₃-C₆ cycloalkyl, and benzyl. In a still further aspect, R₃ is selected from H, methyl, ethyl, n-propyl, isopropyl, cyclopropyl, cyclobutyl, cyclopentyl, and benzyl. In yet a further aspect, R₃ is selected from H, methyl, ethyl, cyclopropyl, cyclobutyl, and benzyl. In an even further aspect, R₃ is selected from H, methyl, cyclopropyl, and benzyl.

In various aspects, R₃ is selected from H and C₁-C₇ straight or branched alkyl. In a further aspect, R₃ is selected from H, methyl, ethyl, n-propyl, isopropyl, tert-butyl, isobutyl, and sec-butyl. In a still further aspect, R₃ is selected from H, methyl, ethyl, n-propyl, and isopropyl. In yet a further aspect, R₃ is selected from H, methyl, and ethyl. In an even further aspect, R₃ is selected from H and methyl.

In various aspects, R₃ is selected from H and C₂-C₇ straight or branched alkyl. In a further aspect, R₃ is selected from H, ethyl, n-propyl, isopropyl, tert-butyl, isobutyl, and sec-butyl. In a still further aspect, R₃ is selected from H, ethyl, n-propyl, and isopropyl. In yet a further aspect, R₃ is selected from H and ethyl.

In various aspects, R₃ is C₁-C₇ straight or branched alkyl. In a further aspect, R₃ is C₁-C₄ straight or branched alkyl. In a still further aspect, R₃ is selected from methyl, ethyl, n-propyl, and isopropyl. In yet a further aspect, R₃ is selected from methyl and ethyl. In an even further aspect, R₃ is ethyl. In a still further aspect, R₃ is methyl.

In various aspects, R₃ is H.

2. Example Compounds

In one aspect, a compound can be present as:

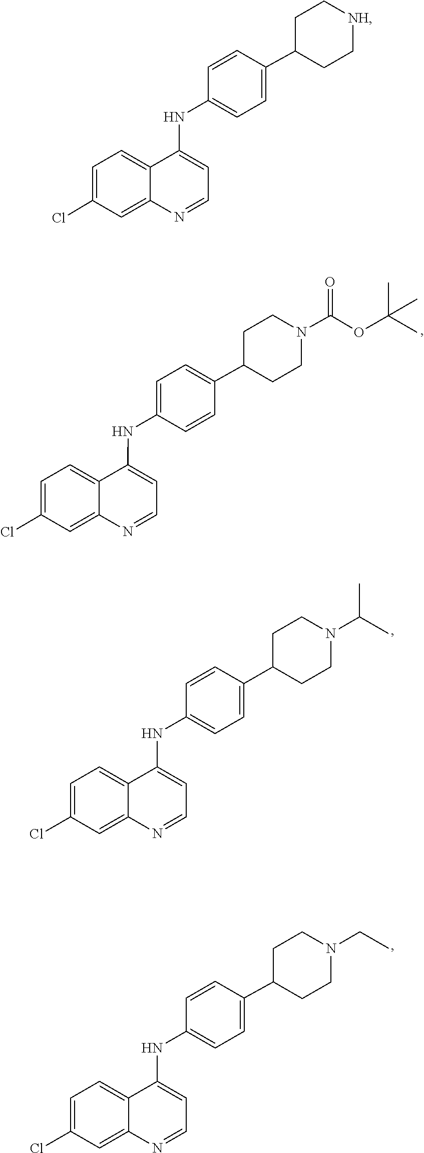 [SMILES: Clc1ccc2c(Nc3ccc(C4CCNCC4)cc3)ccnc2c1 ; CC(C)(C)OC(=O)N1CCC(c2ccc(Nc3ccnc4cc(Cl)ccc34)cc2)CC1 ; CC(C)N1CCC(c2ccc(Nc3ccnc4cc(Cl)ccc34)cc2)CC1 ; CCN1CCC(c2ccc(Nc3ccnc4cc(Cl)ccc34)cc2)CC1]

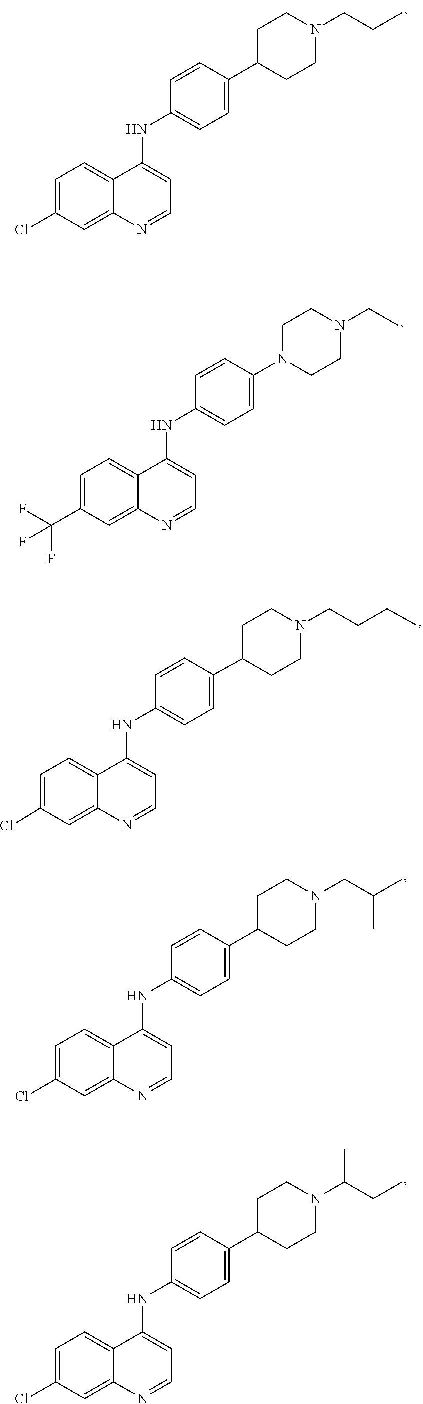 [SMILES: CCCN1CCC(c2ccc(Nc3ccnc4cc(Cl)ccc34)cc2)CC1 ; CCN1CCN(c2ccc(Nc3ccnc4cc(C(F)(F)F)ccc34)cc2)CC1 ; CCCCN1CCC(c2ccc(Nc3ccnc4cc(Cl)ccc34)cc2)CC1 ; CC(C)CN1CCC(c2ccc(Nc3ccnc4cc(Cl)ccc34)cc2)CC1 ; CCC(C)N1CCC(c2ccc(Nc3ccnc4cc(Cl)ccc34)cc2)CC1]

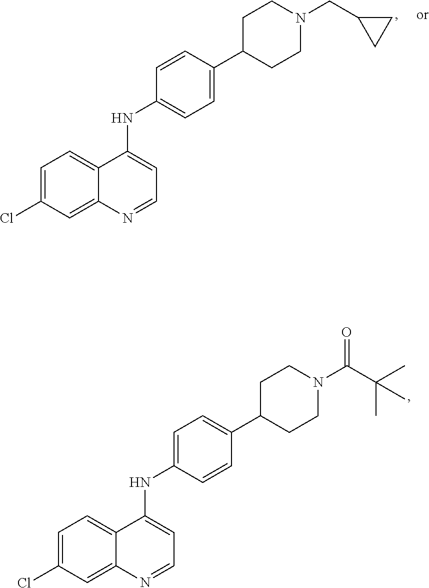 [SMILES: Clc1ccc2c(Nc3ccc(C4CCN(CC5CC5)CC4)cc3)ccnc2c1 ; CC(C)(C)C(=O)N1CCC(c2ccc(Nc3ccnc4cc(Cl)ccc34)cc2)CC1]

or a pharmaceutically acceptable salt thereof.

In one aspect, a compound can be present as:

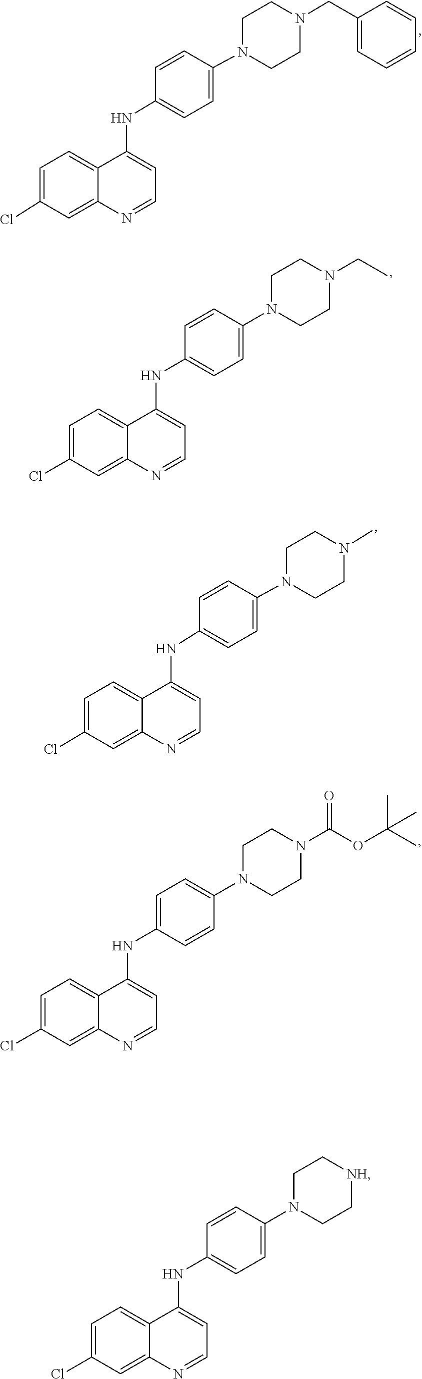 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1]

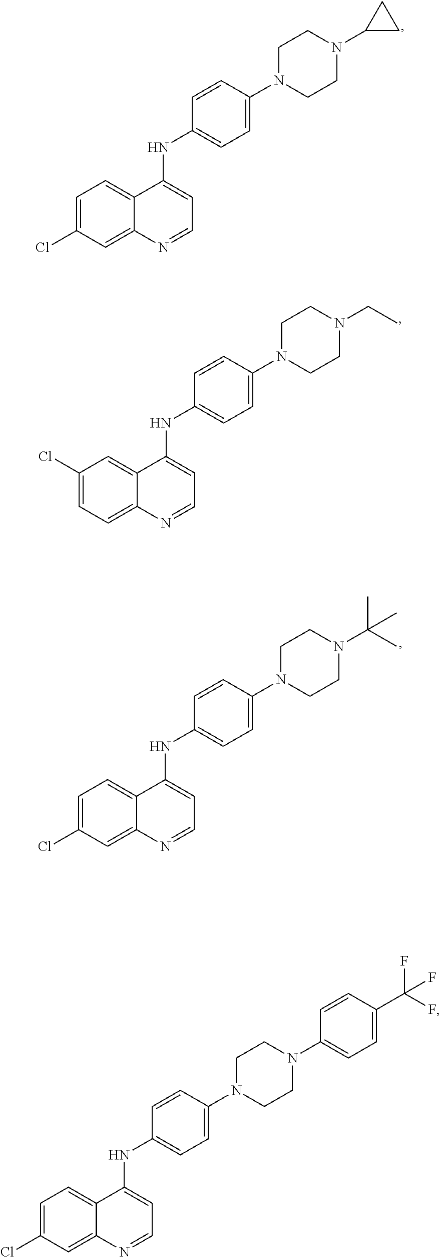 [SMILES: Clc1ccc2c(Nc3ccc(N4CCN(C5CC5)CC4)cc3)ccnc2c1 ; CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1]

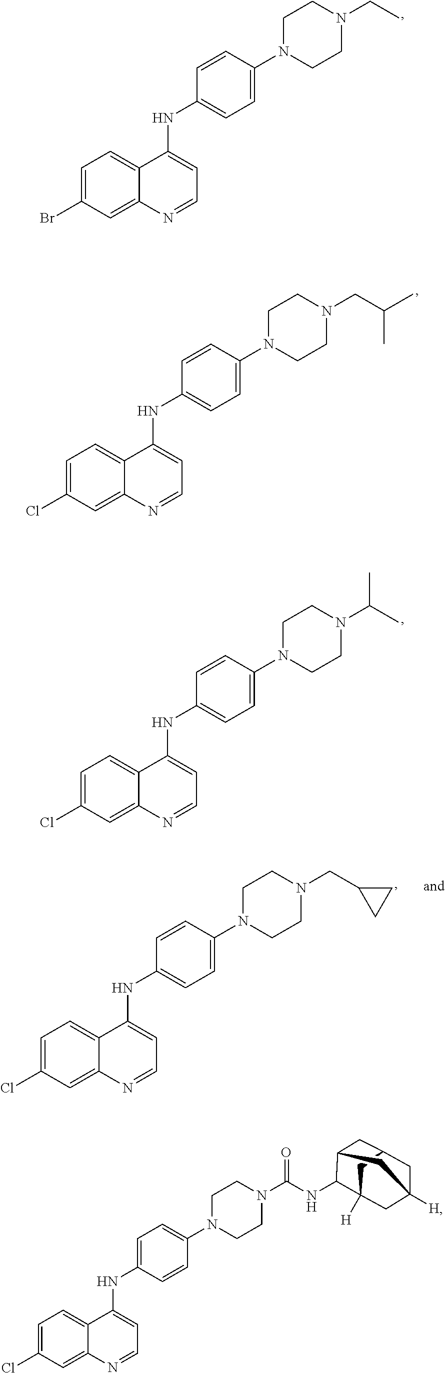

or a pharmaceutically acceptable salt thereof.

### C. Pharmaceutical Compositions

In one aspect, disclosed are pharmaceutical compositions comprising a pharmaceutically acceptable carrier or excipient and a pharmaceutically effective amount of a disclosed compound or a pharmaceutically acceptable salt thereof.

Thus, in one aspect, disclosed are pharmaceutical compositions comprising a pharmaceutically acceptable carrier or excipient and a pharmaceutically effective amount of a compound of Formula (I), or a pharmaceutically acceptable salt thereof:

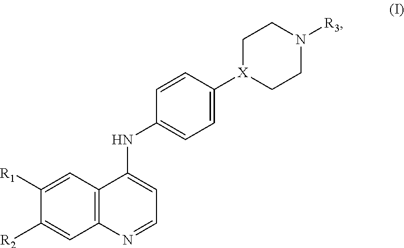

wherein: R₁ is selected from H and halogen; R₂ is selected from H, halogen, and halomethyl; R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and X is selected from N and C; with the proviso that, when R₁ is H, R₂ is not H; with the proviso that, when R₂ is H, R₁ is not H; and with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof.

In one aspect, disclosed are pharmaceutical compositions comprising a pharmaceutically acceptable carrier or excipient and a pharmaceutically effective amount of a compound selected from:

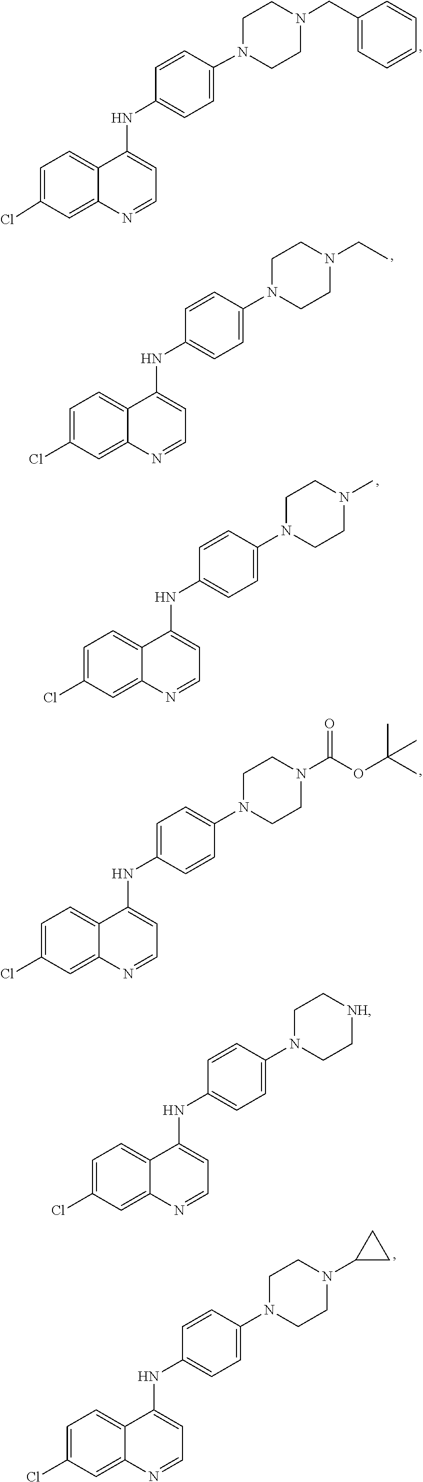 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1 ; Clc1ccc2c(Nc3ccc(N4CCN(C5CC5)CC4)cc3)ccnc2c1]

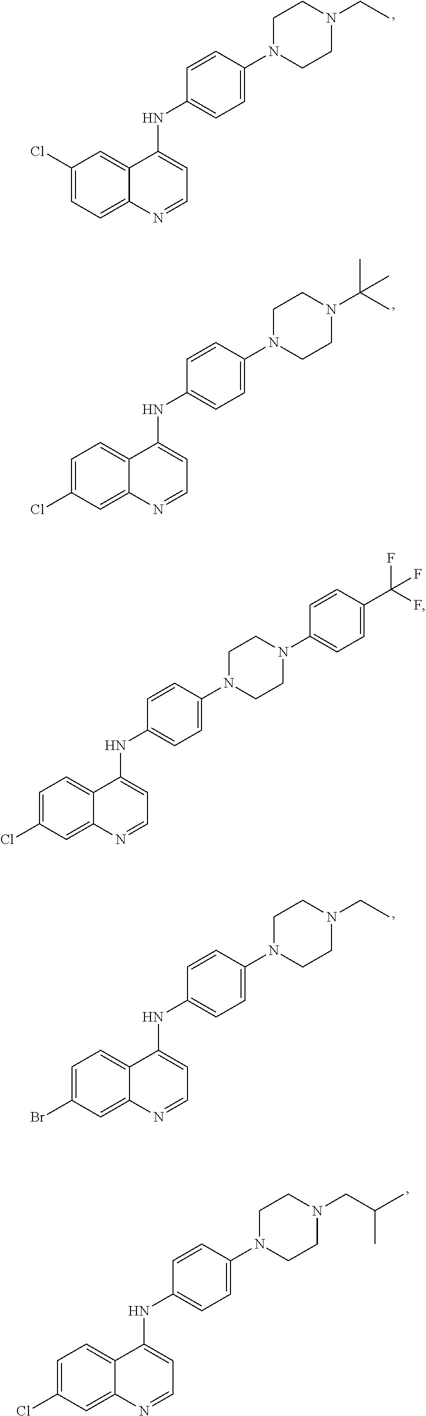 [SMILES: CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1 ; CCN1CCN(c2ccc(Nc3ccnc4cc(Br)ccc34)cc2)CC1 ; CC(C)CN1CCN(c2ccc(Nc3ccnc4cc(Cl)ccc34)cc2)CC1]

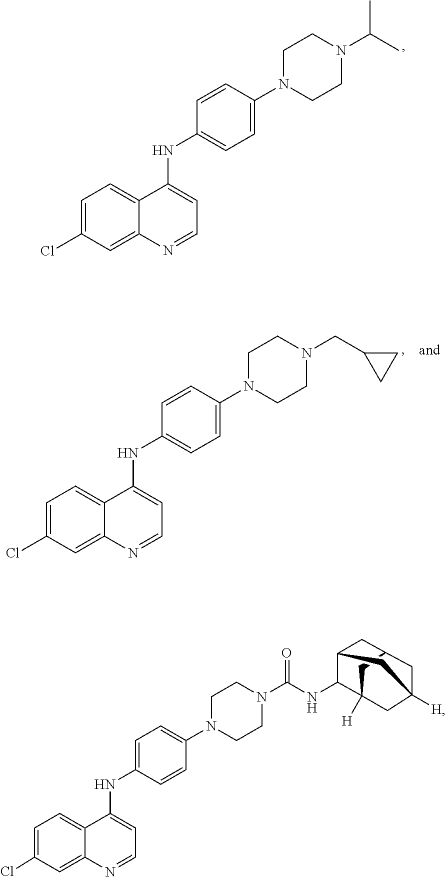

or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, P A 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouthwashes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the effective amount is a therapeutically effective amount. In a still further aspect, the effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat malaria. In a still further aspect, the malaria is a drug-resistant malaria. In yet a further aspect, malaria is a multidrug-resistant malaria.

It is understood that the disclosed compositions can be prepared from the disclosed compounds. It is also understood that the disclosed compositions can be employed in the disclosed methods of using.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-IV, as described and exemplified below. The following examples are provided so that the invention might be more fully understood, are illustrative only, and should not be construed as limiting.

1. Route

In one aspect, substituted N-(4-(piperazin-1-yl)phenyl)quinolin-4-amines can be prepared as shown below.

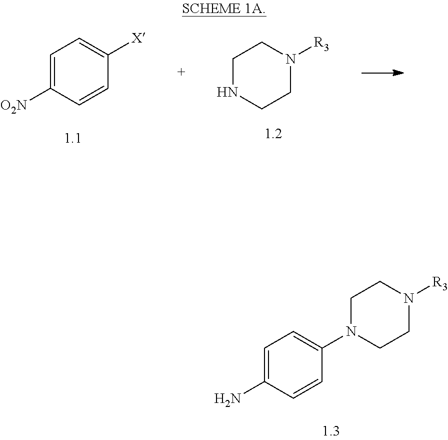

Compounds are represented in generic form, wherein X′ is a halogen and with other substituents as noted in compound descriptions elsewhere herein. A more specific example is set forth below.

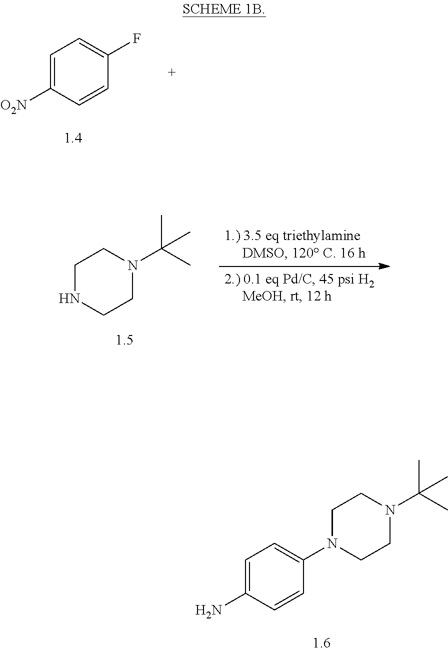

In one aspect, compounds of type 1.6, and similar compounds, can be prepared according to reaction Scheme 1B above. Thus, compounds of type 1.6 can be prepared by a coupling reaction between an appropriate 1-halo-4-nitrobenzene, e.g., 1.4 as shown above, and an appropriate piperazine, e.g., 1.5 as shown above, followed by a reduction. Appropriate 1-halo-4-nitrobenzenes and appropriate piperazines are commercially available or prepared by methods known to one skilled in the art. The coupling reaction is carried out in the presence of an appropriate base, e.g., triethylamine, in an appropriate solvent, e.g., dimethylsulfoxide (DMSO), at an appropriate temperature, e.g., 120° C., for an appropriate period of time, e.g., 16 hours. The reduction is carried out in the presence of an appropriate catalyst, e.g., palladium on carbon, and an appropriate hydrogen source, e.g., hydrogen gas, in an appropriate protic solvent, e.g., methanol (MeOH). As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted N-(4-(piperazin-1-yl)phenyl)quinolin-4-amines similar to Formula 1.2.

2. Route II

In one aspect, substituted N-(4-(piperidin-4-yl)phenyl)quinolin-4-amines and N-(4-(piperazin-1-yl)phenyl)quinolin-4-amines can be prepared as shown below.

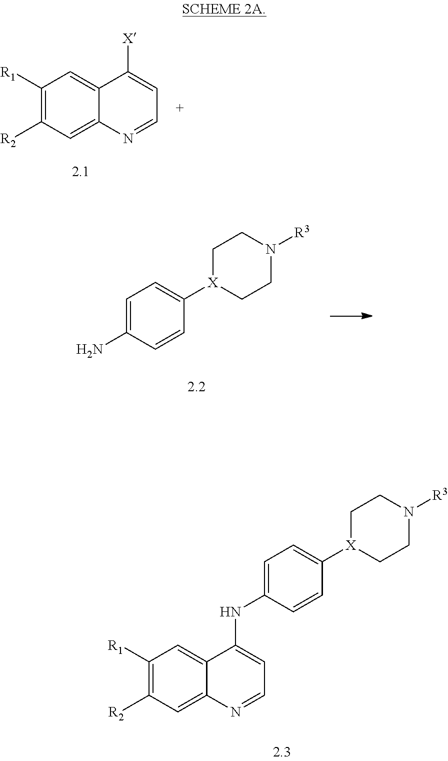

Compounds are represented in generic form, wherein X′ is a halogen and with other substituents as noted in compound descriptions elsewhere herein. A more specific example is set forth below.

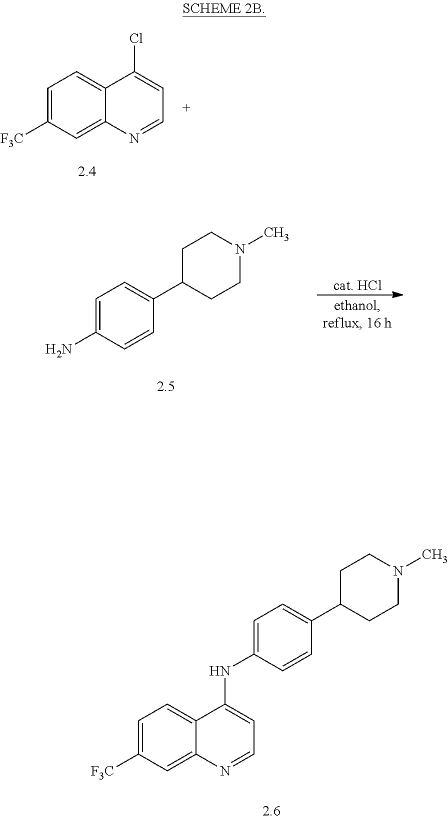 [SMILES: FC(F)(F)c1ccc2c(Cl)ccnc2c1 ; CN1CCC(c2ccc(N)cc2)CC1 ; CN1CCC(c2ccc(Nc3ccnc4cc(C(F)(F)F)ccc34)cc2)CC1]

In one aspect, compounds of type 2.6, and similar compounds, can be prepared according to reaction Scheme 2B above. Thus, compounds of type 2.6 can be prepared by a coupling reaction between an appropriate 4-haloquinoline, e.g., 2.4 as shown above, and an appropriate aniline, e.g., 2.5 as shown above. Appropriate 4-haloquinolines and appropriate anilines are commercially available or prepared by methods known to one skilled in the art. The coupling reaction is carried out in the presence of an appropriate acid, e.g., hydrochloric acid, in an appropriate solvent, e.g., ethanol, at an appropriate temperature, e.g., reflux, for an appropriate period of time, e.g., 16 hours. Alternative coupling conditions are known in the art and also described elsewhere herein. See, e.g., General Procedures B and D. As can be appreciated by one skilled in the art, the above reaction provides an example of a generalized approach wherein compounds similar in structure to the specific reactants above (compounds similar to compounds of type 2.1 and 2.2), can be substituted in the reaction to provide substituted N-(4-(piperidin-4-yl)phenyl)quinolin-4-amines and N-(4-(piperazin-1-yl)phenyl)quinolin-4-amines similar to Formula 2.3.

3. Route III

In one aspect, substituted N-(4-(piperidin-4-yl)phenyl)quinolin-4-amines and N-(4-(piperazin-1-yl)phenyl)quinolin-4-amines can be prepared as shown below.

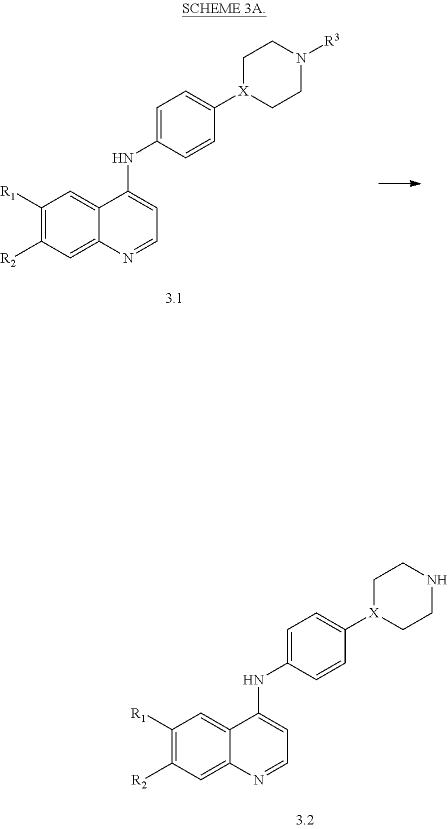

Compounds are represented in generic form, wherein R₃ is a —C(═O)—C₁-C₇ straight or branched alkyl and with other substituents as noted in compound descriptions elsewhere herein. A more specific example is set forth below.

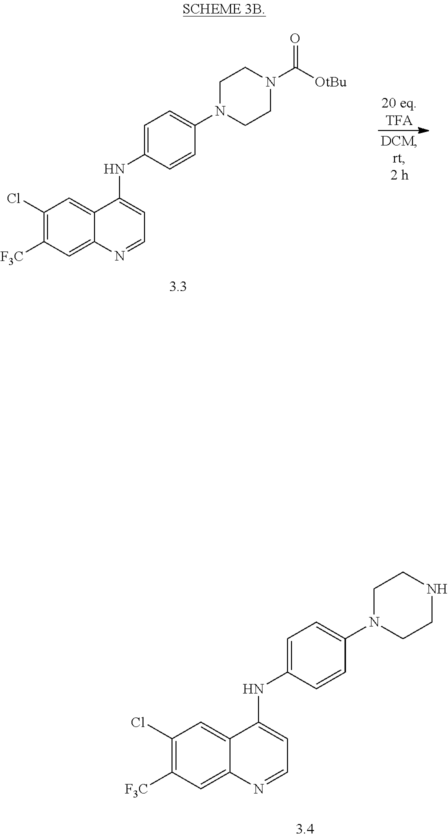 [SMILES: CC(C)(C)OC(=O)N1CCN(c2ccc(Nc3ccnc4cc(C(F)(F)F)c(Cl)cc34)cc2)CC1 ; FC(F)(F)c1cc2nccc(Nc3ccc(N4CCNCC4)cc3)c2cc1Cl]

In one aspect, compounds of type 3.4, and similar compounds, can be prepared according to reaction Scheme 3B above. Thus, compounds of type 3.4 can be prepared by deprotection of an appropriate carbamate, e.g., 3.3 as shown above. The deprotection is carried out in the presence of an appropriate acid, e.g., trifluoroacetic acid (TFA), in an appropriate solvent, e.g., dichloromethane (DCM). As can be appreciated by one skilled in the art, the above reaction provides an example of a generalized approach wherein compounds similar in structure to the specific reactants above (compounds similar to compounds of type 3.1), can be substituted in the reaction to provide substituted N-(4-(piperidin-4-yl)phenyl)quinolin-4-amines and N-(4-(piperazin-1-yl)phenyl)quinolin-4-amines similar to Formula 3.2.

4. Route IV

In one aspect, substituted N-(4-(piperidin-4-yl)phenyl)quinolin-4-amines and N-(4-(piperazin-1-yl)phenyl)quinolin-4-amines can be prepared as shown below.

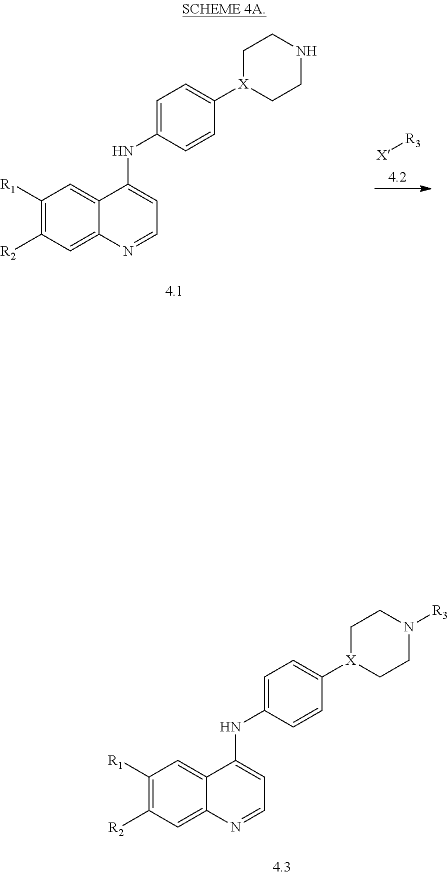

Compounds are represented in generic form, wherein X′ is a halogen and with other substituents as noted in compound descriptions elsewhere herein. A more specific example is set forth below.

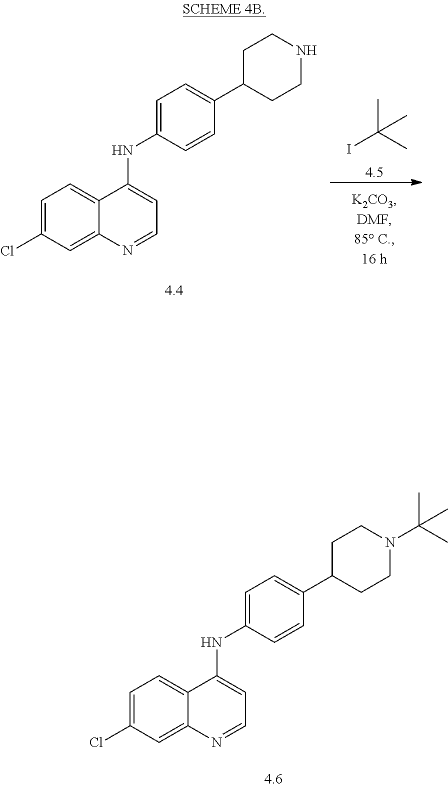

In one aspect, compounds of type 4.6, and similar compounds, can be prepared according to reaction Scheme 4B above. Thus, compounds of type 4.6 can be prepared by alkylation of an appropriate piperidine or an appropriate piperazine, e.g., 4.4 as shown above. The alkylation is carried out in the presence of an appropriate alkyl halide, e.g., 4.5 as shown above, and an appropriate base, e.g., potassium carbonate, in an appropriate solvent, e.g., dimethylformamide (DMF), at an appropriate temperature, e.g., 85° C., for an appropriate period of time, e.g., 16 hours. As can be appreciated by one skilled in the art, the above reaction provides an example of a generalized approach wherein compounds similar in structure to the specific reactants above (compounds similar to compounds of type 4.1 and 4.2), can be substituted in the reaction to provide substituted N-(4-(piperidin-4-yl)phenyl)quinolin-4-amines and N-(4-(piperazin-1-yl)phenyl)quinolin-4-amines similar to Formula 4.3.

### E. Methods for Treating Malaria

In one aspect, disclosed are methods of treating malaria in a subject in need thereof, the method comprising administering to the subject an effective amount of a disclosed compound or a pharmaceutically acceptable salt thereof.

Thus, in one aspect, disclosed are methods of treating malaria in a subject in need thereof, the method comprising administering to the subject a pharmaceutically effective amount of a compound of Formula (I), or a pharmaceutically acceptable salt thereof:

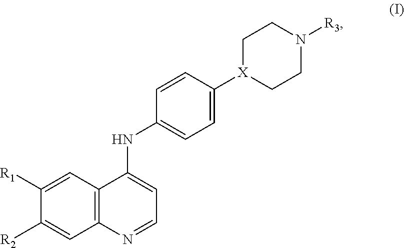

wherein: R₁ is selected from H and halogen; R₂ is selected from H, halogen, and halomethyl; R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and X is selected from N and C; with the proviso that, when R₁ is H, R₂ is not H; with the proviso that, when R₂ is H, R₁ is not H; and with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof.

In one aspect, disclosed are methods of treating malaria in a subject in need thereof, the method comprising administering to the subject a pharmaceutically effective amount of a compound selected from:

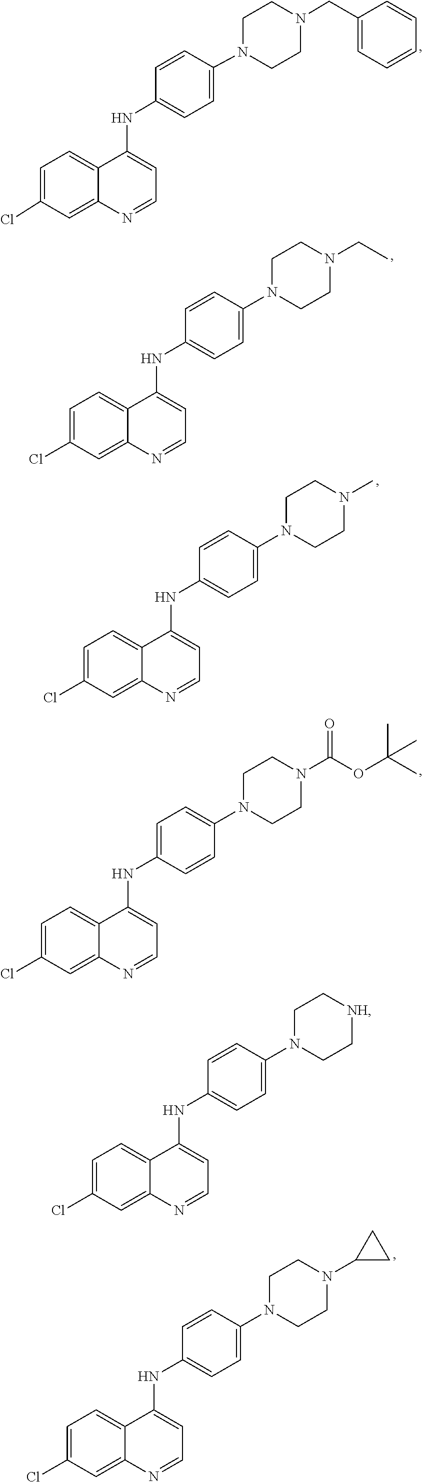 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1 ; Clc1ccc2c(Nc3ccc(N4CCN(C5CC5)CC4)cc3)ccnc2c1]

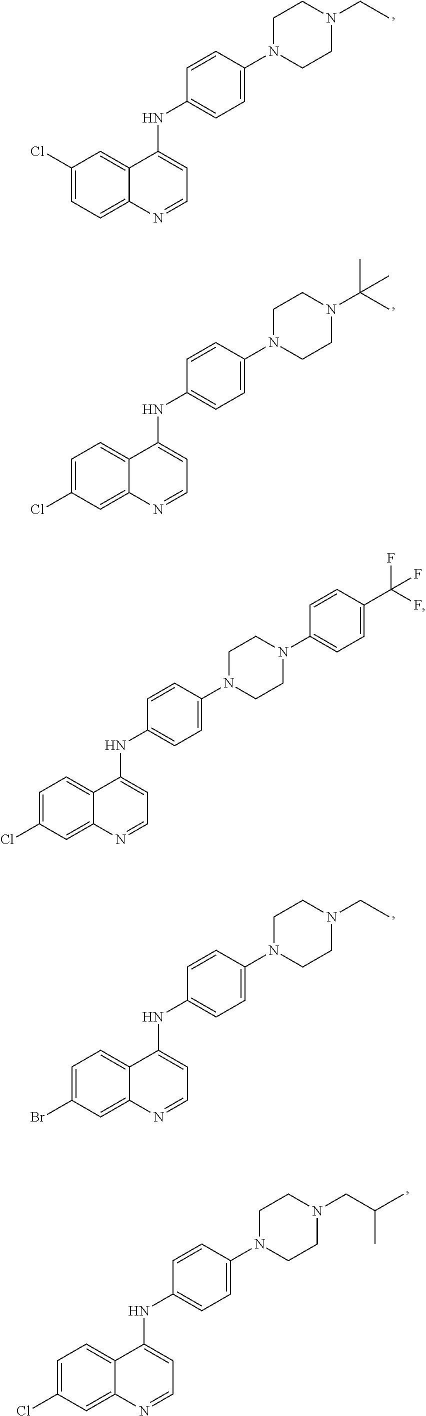 [SMILES: CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1 ; CCN1CCN(c2ccc(Nc3ccnc4cc(Br)ccc34)cc2)CC1 ; CC(C)CN1CCN(c2ccc(Nc3ccnc4cc(Cl)ccc34)cc2)CC1]

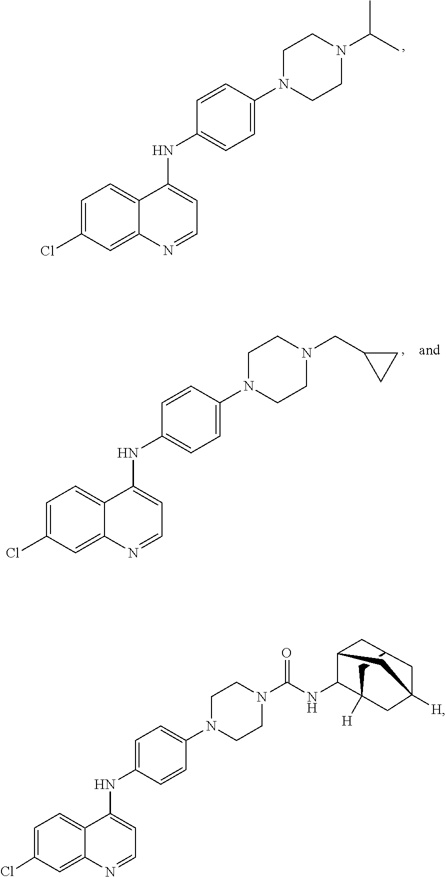

or a pharmaceutically acceptable salt thereof.

In various aspects, X is N; or a pharmaceutically acceptable salt thereof.

In various aspects, X is C; or a pharmaceutically acceptable salt thereof.

In various aspects, R₃ is selected from H, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; or a pharmaceutically acceptable salt thereof.

In various aspects, R₃ is H; or a pharmaceutically acceptable salt thereof.

In various aspects, R₃ is —C(═O)—C₁-C₇ straight or branched alkyl; or a pharmaceutically acceptable salt thereof.

In various aspects, R₃ is selected from C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; or a pharmaceutically acceptable salt thereof.

In various aspects, the compound, or a pharmaceutically acceptable salt thereof, has a Formula (I):

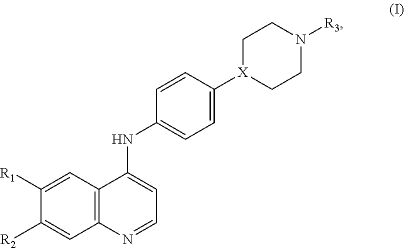

wherein: R₁ is selected from H and halogen; R₂ is selected from H, halogen, and halomethyl; R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and X is selected from N and C; with the proviso that one or more selected from: (a) X is C and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; (b) R₁ is halogen and R₂ is selected from H and halomethyl; and (c) R₂ is halomethyl and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl.

In various aspects, the compound, or a pharmaceutically acceptable salt thereof, has a Formula (VI):

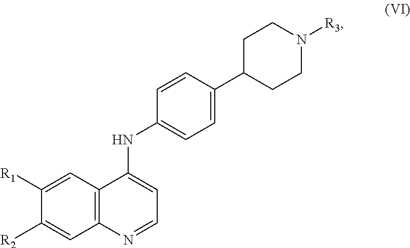

wherein: R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl. In a further aspect, R₁ is halogen and R₂ is selected from H and halomethyl; or a pharmaceutically acceptable salt thereof. In a still further aspect, R₂ is halomethyl and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; or a pharmaceutically acceptable salt thereof. In yet a further aspect, R₃ is selected from the group of H, C₁-C₇ straight or branched alkyl, C₃-C₆ cycloalkyl, —CH₂—C₃-C₆ cycloalkyl, and benzyl.

In various aspects, R₁ is selected from H and halogen; R₂ is selected from H, halogen, and halomethyl; and R₃ is selected from H, C₁-C₇ straight or branched alkyl, C₃-C₆ cycloalkyl, —CH₂—C₃-C₆ cycloalkyl, and benzyl; X is selected from N and C; with the proviso that, when R₁ is H, R₂ is not H; and with the proviso that, when R₂ is H, R₁ is not H.

In various aspects, R₁ is selected from H and C₁; R₂ is selected from H, Cl, and fluoromethyl; and R₃ is selected from H, C₁-C₇ straight or branched alkyl, C₃-C₆ cycloalkyl, —CH₂—C₃-C₆ cycloalkyl, and benzyl; X is selected from N and C; with the proviso that, when R₁ is H, R₂ is not H; and with the proviso that, when R₂ is H, R₁ is not H.

In various aspects, R₁ is H; R₂ is selected from C₁ and CF₃; and R₃ is selected from H, C₁-C₇ straight or branched alkyl, C₃-C₆ cycloalkyl, —CH₂—C₃-C₆ cycloalkyl, and benzyl; and X is selected from N and C.

In various aspects, the compound of Formula (I) is a compound of Formula (II):

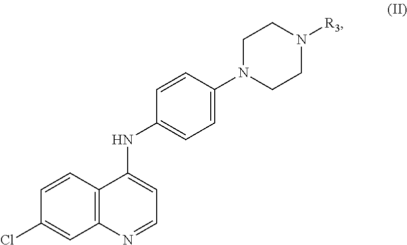

wherein R₃ is selected from H, C₁-C₇ straight or branched alkyl, C₃-C₆ cycloalkyl, —CH₂—C₃-C₆ cycloalkyl, and benzyl. In a further aspect, R₃ is selected from H, ethyl, n-propyl, isopropyl, tert-butyl, isobutyl, sec-butyl, n-pentyl, pentan-2-yl, 2-methylbutyl, isopentyl, 3-methylbutan-2-yl, n-hexyl, isohexyl, t-hexyl, sec-hexyl, 2-methylpentyl, 3-methylpentyl, n-heptyl, 5-methylhexyl, t-heptyl, sec-heptyl, and iso-heptyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof. In a still further aspect, R₃ is selected from H, ethyl, n-propyl, isopropyl, tert-butyl, isobutyl, and sec-butyl. In a yet further aspect, R₃ is selected from H, C₁-C₄ straight or branched alkyl, C₃-C₆ cycloalkyl, —CH₂—C₃-C₆ cycloalkyl, and benzyl. In an even further aspect, R₃ is selected from H, C₁-C₄ straight or branched alkyl, C₃-C₆ cycloalkyl, and benzyl. In a still further aspect, R₃ is C₁-C₄ straight or branched alkyl.

In various aspects, the compound of Formula (I) is a compound of Formula (III):

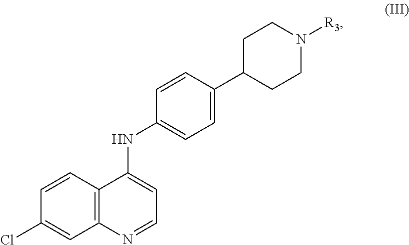

wherein R₃ is selected from H and C₂-C₇ straight or branched alkyl; or a pharmaceutically acceptable salt thereof.

In various aspects, the compound is selected from:

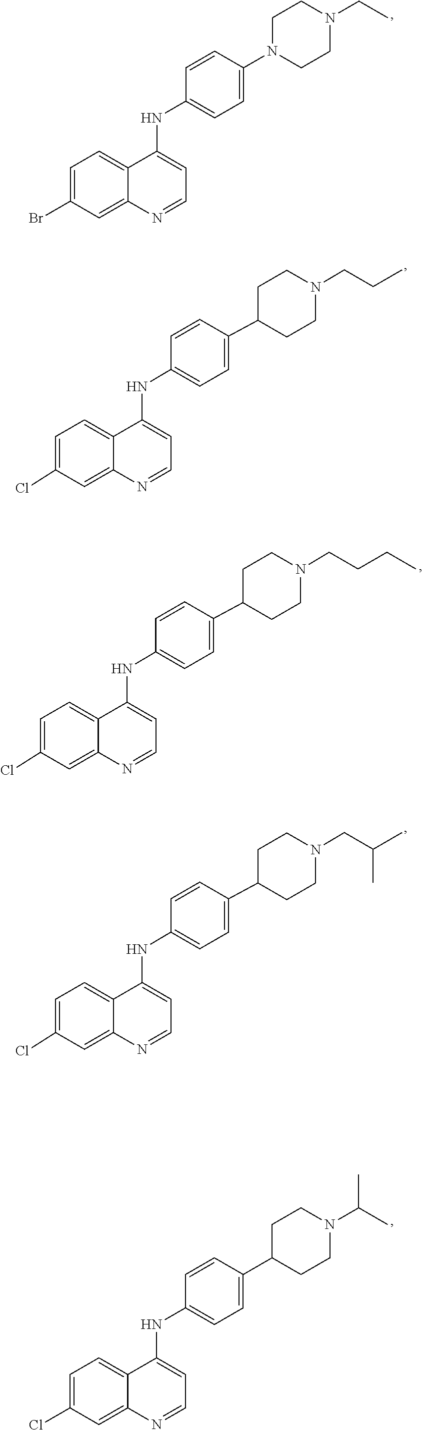 [SMILES: CCN1CCN(c2ccc(Nc3ccnc4cc(Br)ccc34)cc2)CC1 ; CCCN1CCC(c2ccc(Nc3ccnc4cc(Cl)ccc34)cc2)CC1 ; CCCCN1CCC(c2ccc(Nc3ccnc4cc(Cl)ccc34)cc2)CC1 ; CC(C)CN1CCC(c2ccc(Nc3ccnc4cc(Cl)ccc34)cc2)CC1 ; CC(C)N1CCC(c2ccc(Nc3ccnc4cc(Cl)ccc34)cc2)CC1]

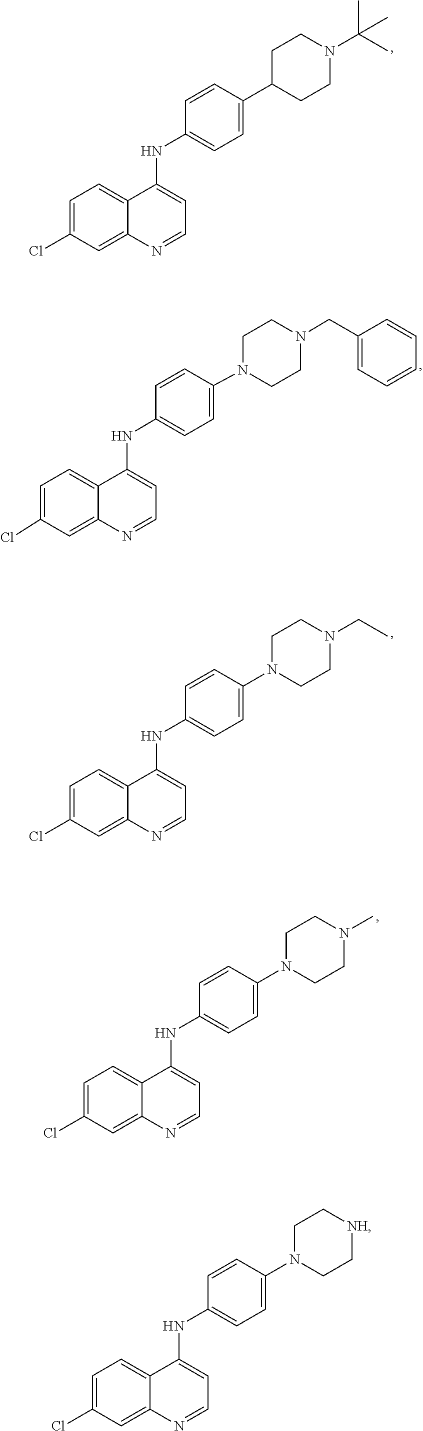 [SMILES: CC(C)(C)N1CCC(c2ccc(Nc3ccnc4cc(Cl)ccc34)cc2)CC1 ; Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; Clc1ccc2c(Nc3ccc(N4CCNCC4)cc3)ccnc2c1]

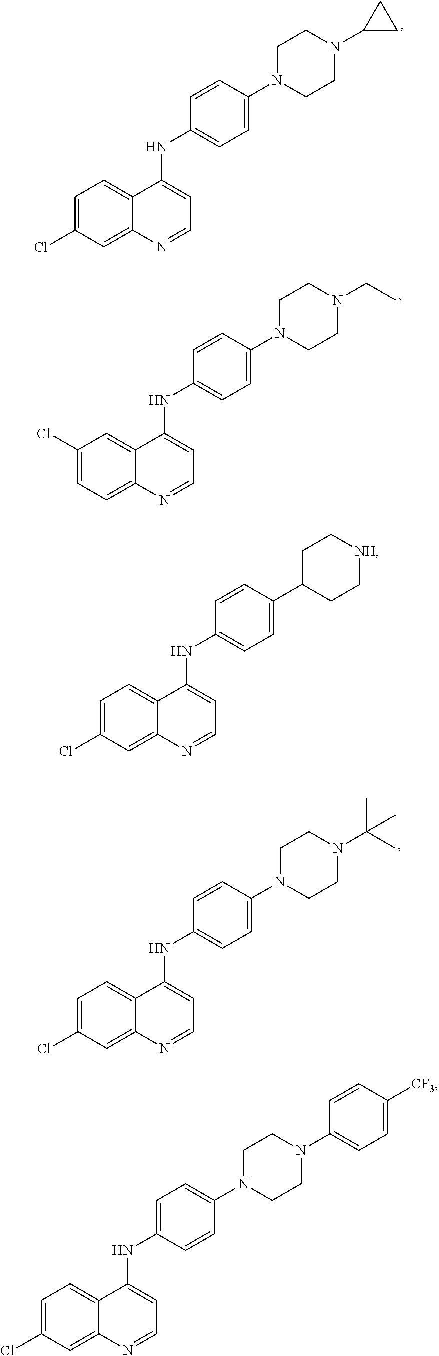 [SMILES: Clc1ccc2c(Nc3ccc(N4CCN(C5CC5)CC4)cc3)ccnc2c1 ; CCN1CCN(c2ccc(Nc3ccnc4ccc(Cl)cc34)cc2)CC1 ; Clc1ccc2c(Nc3ccc(C4CCNCC4)cc3)ccnc2c1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1]

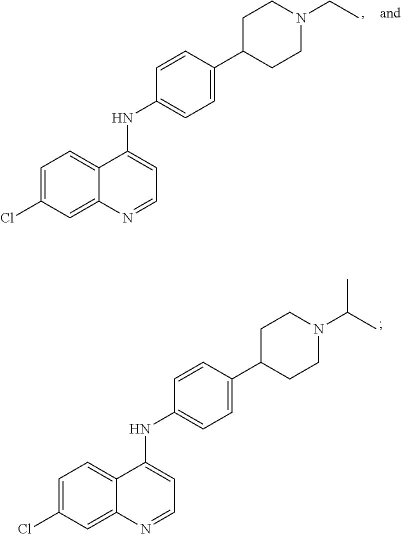 [SMILES: CCN1CCC(c2ccc(Nc3ccnc4cc(Cl)ccc34)cc2)CC1 ; CC(C)N1CCC(c2ccc(Nc3ccnc4cc(Cl)ccc34)cc2)CC1]

or a pharmaceutically acceptable salt thereof.

In various aspects, the compound of Formula (I) is selected from:

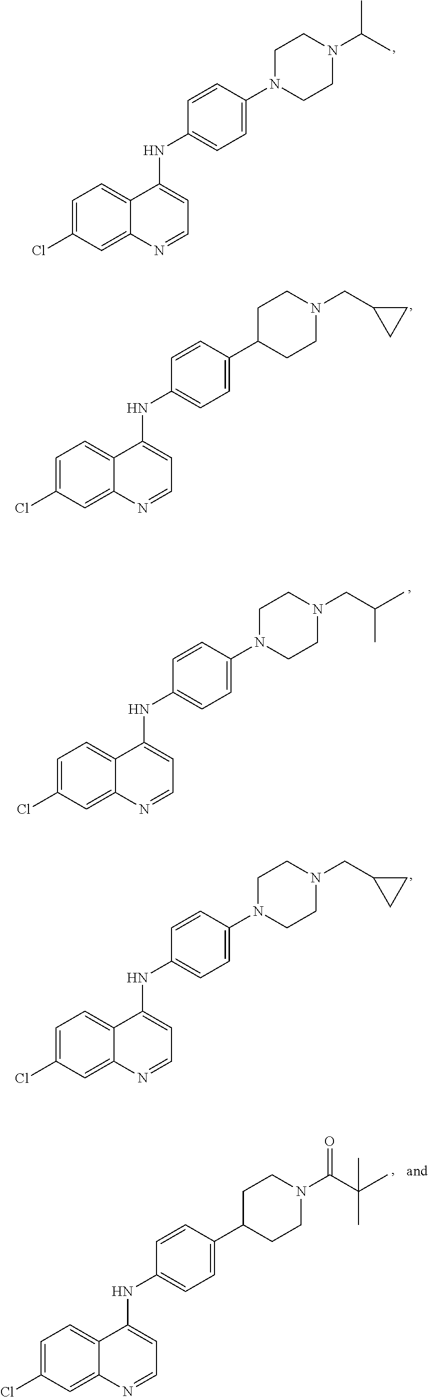 [SMILES: CC(C)N1CCN(c2ccc(Nc3ccnc4cc(Cl)ccc34)cc2)CC1 ; Clc1ccc2c(Nc3ccc(C4CCN(CC5CC5)CC4)cc3)ccnc2c1 ; CC(C)CN1CCN(c2ccc(Nc3ccnc4cc(Cl)ccc34)cc2)CC1 ; Clc1ccc2c(Nc3ccc(N4CCN(CC5CC5)CC4)cc3)ccnc2c1 ; CC(C)(C)C(=O)N1CCC(c2ccc(Nc3ccnc4cc(Cl)ccc34)cc2)CC1]

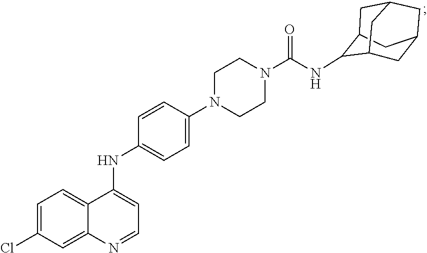 [SMILES: O=C(NC1C2CC3CC(C2)CC1C3)N1CCN(c2ccc(Nc3ccnc4cc(Cl)ccc34)cc2)CC1]

or a pharmaceutically acceptable salt thereof.

In various aspects, the compound is selected from:

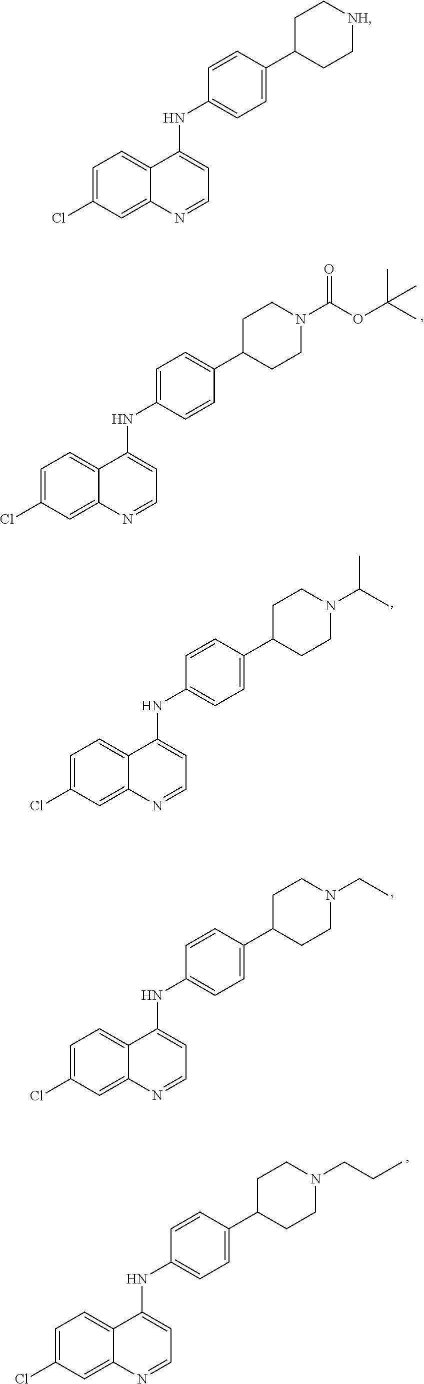 [SMILES: Clc1ccc2c(Nc3ccc(C4CCNCC4)cc3)ccnc2c1 ; CC(C)(C)OC(=O)N1CCC(c2ccc(Nc3ccnc4cc(Cl)ccc34)cc2)CC1 ; CC(C)N1CCC(c2ccc(Nc3ccnc4cc(Cl)ccc34)cc2)CC1 ; CCN1CCC(c2ccc(Nc3ccnc4cc(Cl)ccc34)cc2)CC1 ; CCCN1CCC(c2ccc(Nc3ccnc4cc(Cl)ccc34)cc2)CC1]

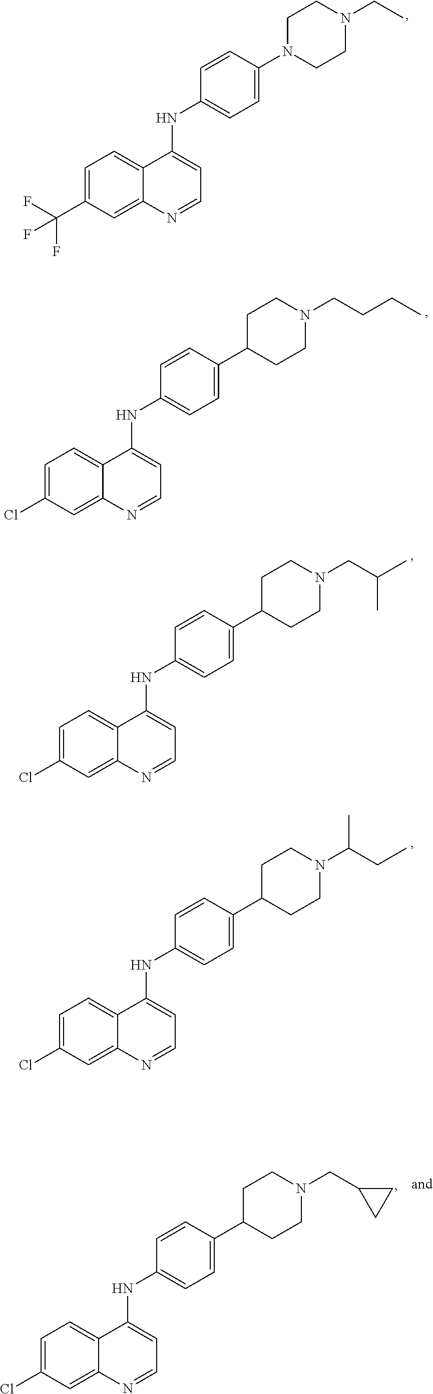 [SMILES: CCN1CCN(c2ccc(Nc3ccnc4cc(C(F)(F)F)ccc34)cc2)CC1 ; CCCCN1CCC(c2ccc(Nc3ccnc4cc(Cl)ccc34)cc2)CC1 ; CC(C)CN1CCC(c2ccc(Nc3ccnc4cc(Cl)ccc34)cc2)CC1 ; CCC(C)N1CCC(c2ccc(Nc3ccnc4cc(Cl)ccc34)cc2)CC1 ; Clc1ccc2c(Nc3ccc(C4CCN(CC5CC5)CC4)cc3)ccnc2c1]

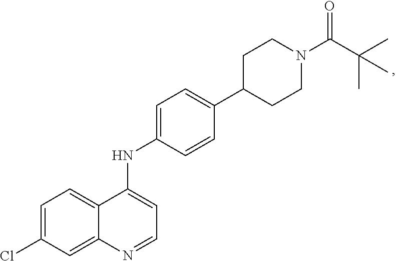 [SMILES: CC(C)(C)C(=O)N1CCC(c2ccc(Nc3ccnc4cc(Cl)ccc34)cc2)CC1]

or a pharmaceutically acceptable salt thereof.

In various aspects, the compound is selected from:

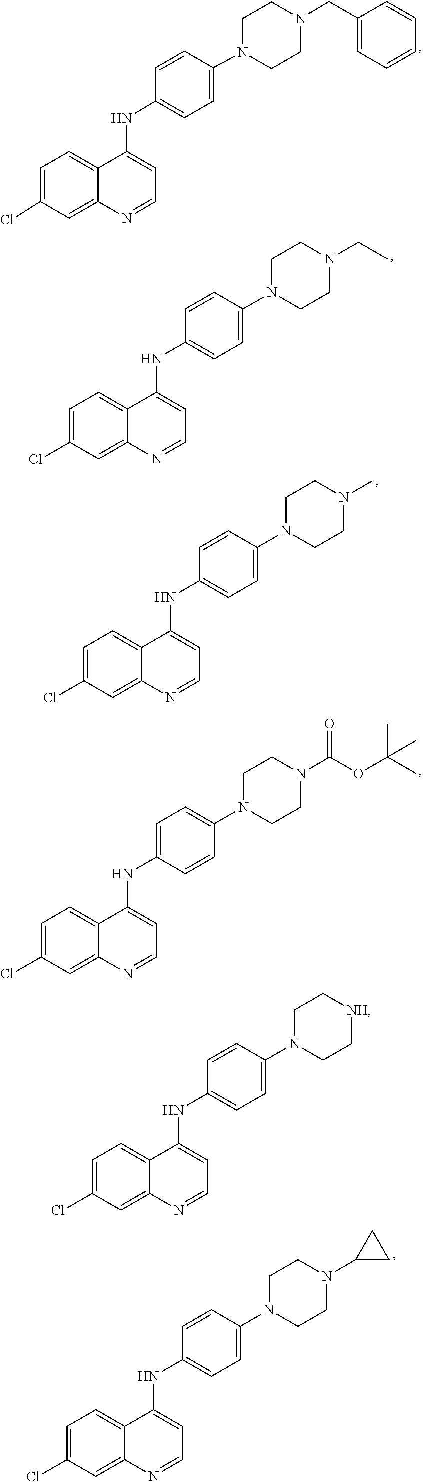 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1 ; Clc1ccc2c(Nc3ccc(N4CCN(C5CC5)CC4)cc3)ccnc2c1]

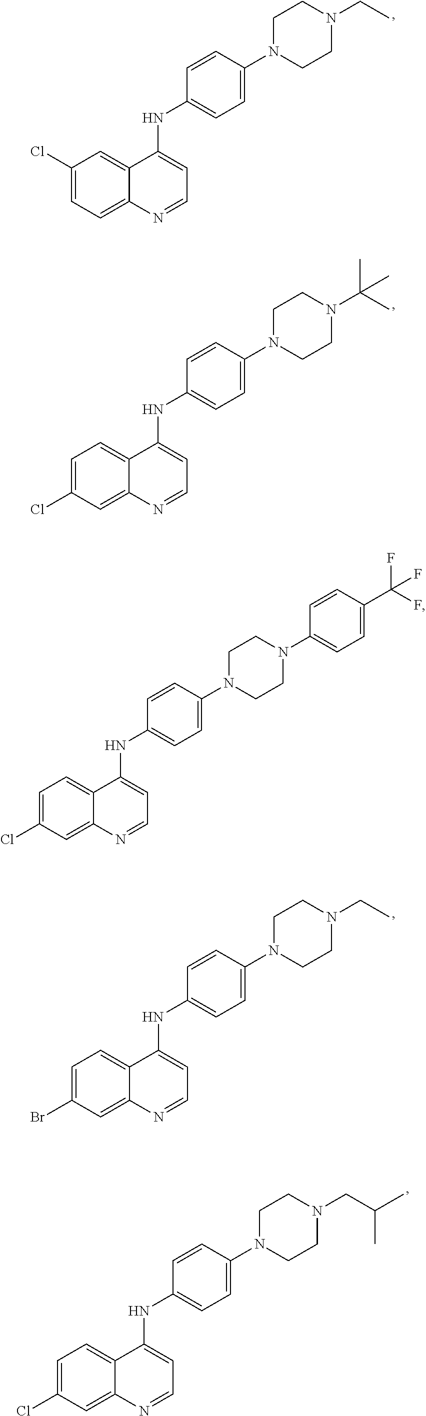 [SMILES: CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1 ; CCN1CCN(c2ccc(Nc3ccnc4cc(Br)ccc34)cc2)CC1 ; CC(C)CN1CCN(c2ccc(Nc3ccnc4cc(Cl)ccc34)cc2)CC1]

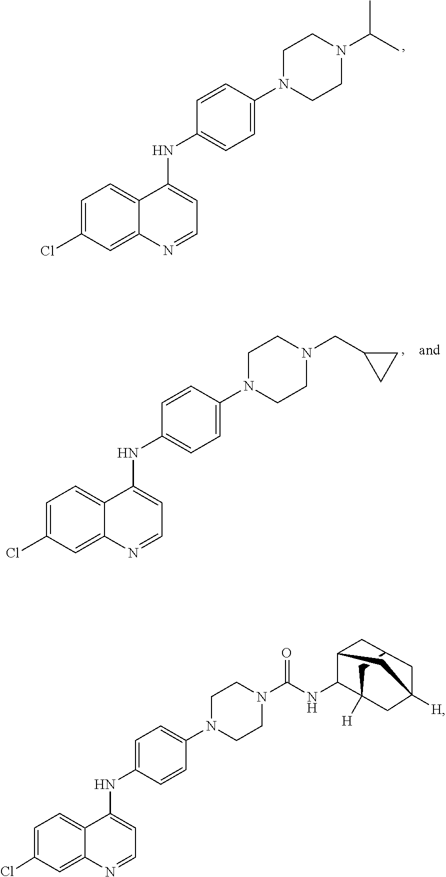

or a pharmaceutically acceptable salt thereof.

In a further aspect, the subject has been diagnosed with a need for treatment of malaria prior to the administering step. In a still further aspect, the subject is at risk for developing malaria prior to the administering step.

In a further aspect, the subject is a mammal. In a still further aspect, the mammal is a human.

In a further aspect, the method further comprises the step of identifying a subject in need of treatment of malaria.

In a further aspect, the effective amount is a therapeutically effective amount. In a still further aspect, the effective amount is a prophylactically effective amount.

In a further aspect, the malaria is a drug-resistant malaria. In a still further aspect, the drug-resistant malaria is resistant to treatment by an antimalarial agent selected from the group of chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, artemisinin derivatives (including dihydroaremisinin (DHA), artesunate, artmether, arteether), artemisinin-based combination therapies (ACT), such as DHA-piperaquine and DHA-piperaquine mefloquine-artesunate.

In a further aspect, the malaria is a multidrug-resistant malaria. In a still further aspect, the multidrug-resistant malaria is resistant to treatment by two or more antimalarial agents selected from the group of chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, artemisinin derivatives (including dihy Srdroaremisinin (DHA), artesunate, artmether, arteether), artemisinin-based combination therapies (ACT), such as DHA-piperaquine and DHA-piperaquine mefloquine-artesunate.

In a further aspect, administering is oral or parental administration. In a still further aspect, the parenteral administration is intravenous, subcutaneous, intramuscular, or via direct injection.

In a further aspect, the method further comprises administering a therapeutically effective amount of an antimalarial agent to the subject. In a still further aspect, the antimalarial agent is administered prior to administration of the compound. In yet a further aspect, the antimalarial agent is administered subsequent to administration of the compound.

In a further aspect, the method further comprises administering to the subject an effective amount of at least one antimalarial agent. Examples of antimalarial agents include, but are not limited to, chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, artemisinin derivatives (including dihy Srdroaremisinin (DHA), artesunate, artmether, arteether), artemisinin-based combination therapies (ACT), such as DHA-piperaquine and DHA-piperaquine mefloquine-artesunate.

In a further aspect, the compound and the agent are administered sequentially. In a still further aspect, the compound and the agent are administered simultaneously.

In a further aspect, the compound and the agent are co-formulated. In a still further aspect, the compound and the agent are co-packaged.

In a further aspect, the compound is administered as a single active agent.

### F. Additional Methods of Using the Compounds

The compounds and pharmaceutical compositions of the invention are useful in treating or controlling malaria (e.g., drug-resistant malaria, multidrug-resistant malaria). To treat or control the condition, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of malaria (e.g., drug-resistant malaria, multidrug-resistant malaria).

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of malaria (e.g., drug-resistant malaria, multidrug-resistant malaria).

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Use of Compounds

In one aspect, the invention relates to the use of a disclosed compound or a product of a disclosed method. In a further aspect, a use relates to the manufacture of a medicament for the treatment of malaria (e.g., drug-resistant malaria, multidrug-resistant malaria).

Also provided are uses of the disclosed compounds and products. In one aspect, the invention relates to use of at least one disclosed compound or a pharmaceutically acceptable salt thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt thereof, wherein a pharmaceutically acceptable carrier is intimately mixed with a therapeutically effective amount of the compound or the product of a disclosed method of making.

In various aspects, the use relates to a treatment of malaria. In one aspect, the use is characterized in that the subject is a human. In one aspect, the use is characterized in that the malaria is a drug-resistant malaria. In one aspect, the use is characterized in that the malaria is a multidrug-resistant malaria.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of malaria in a mammal. In a further aspect, the malaria is a drug-resistant malaria. In a still further aspect, the malaria is a multidrug-resistant malaria.

2. Manufacture of a Medicament

In one aspect, the invention relates to a method for the manufacture of a medicament for treating malaria in a subject having the condition, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of malaria (e.g., drug-resistant malaria, multidrug-resistant malaria). The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight for humans, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt thereof, with a pharmaceutically acceptable carrier or diluent.

3. Kits

In one aspect, disclosed are kits comprising a disclosed compound, and one or more selected from: (a) an antimalarial agent; (b) instructions for administering the compound in connection with treating malaria; and (c) instructions for treating malaria.

Thus, in one aspect, disclosed are kits comprising a compound of Formula (I), or a pharmaceutically acceptable salt thereof:

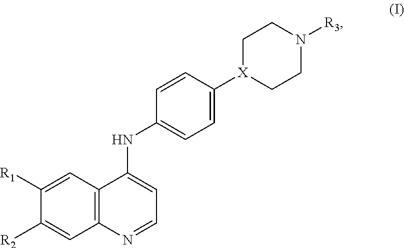

wherein: R₁ is selected from H and halogen; R₂ is selected from H, halogen, and halomethyl; R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and X is selected from N and C; with the proviso that, when R₁ is H, R₂ is not H; with the proviso that, when R₂ is H, R₁ is not H; and with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof, and one or more selected from: (a) an antimalarial agent; (b) instructions for administering the compound in connection with treating malaria; and (c) instructions for treating malaria.

Also disclosed are kits comprising a compound selected from:

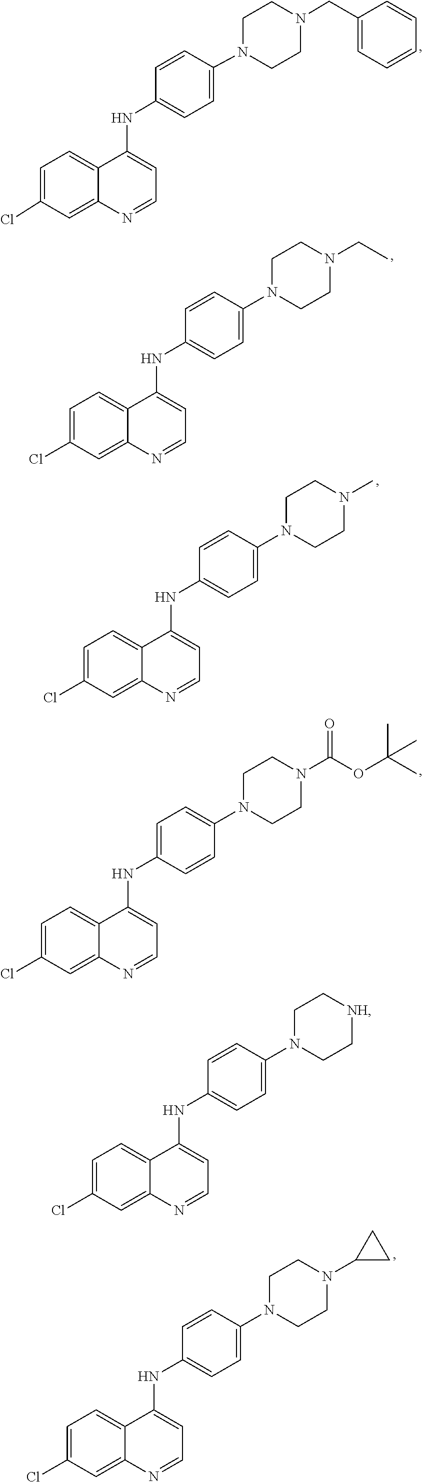 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1 ; Clc1ccc2c(Nc3ccc(N4CCN(C5CC5)CC4)cc3)ccnc2c1]

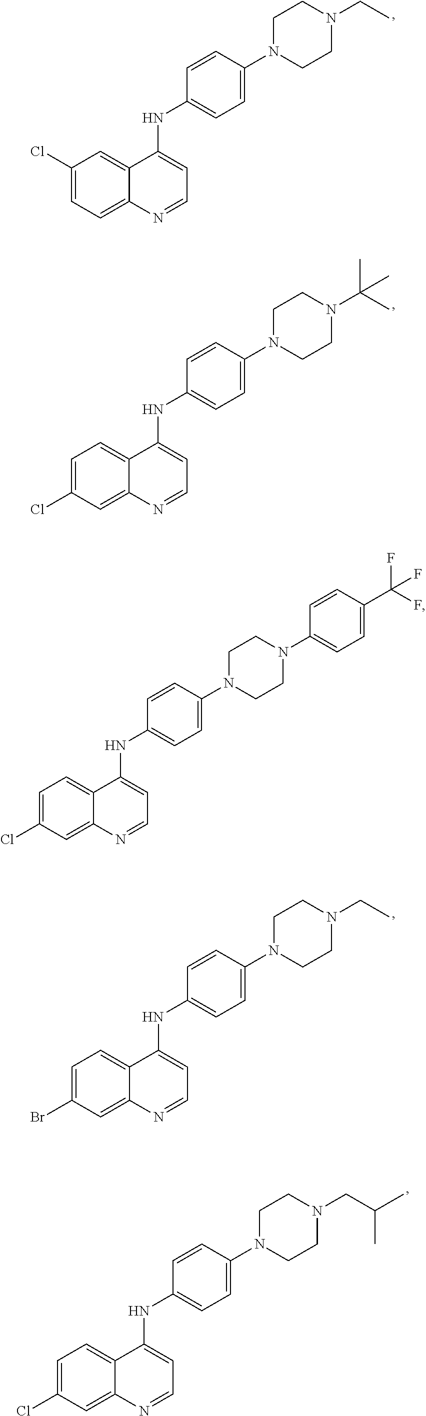 [SMILES: CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1 ; CCN1CCN(c2ccc(Nc3ccnc4cc(Br)ccc34)cc2)CC1 ; CC(C)CN1CCN(c2ccc(Nc3ccnc4cc(Cl)ccc34)cc2)CC1]

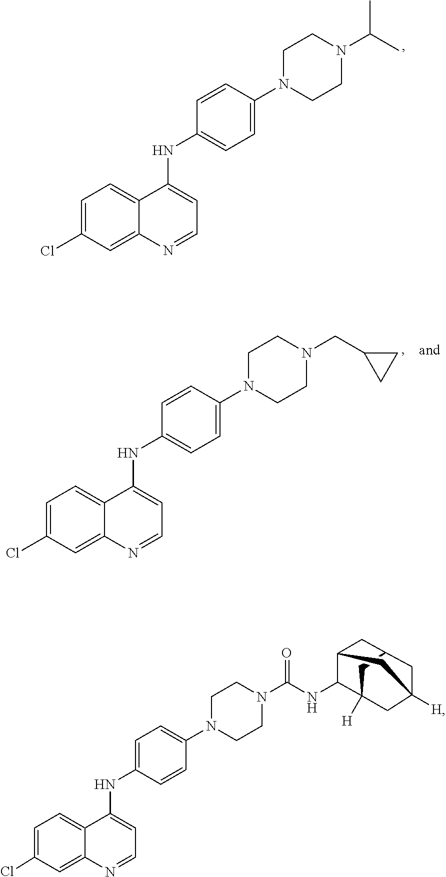

or a pharmaceutically acceptable salt thereof, and one or more selected from: (a) an antimalarial agent; (b) instructions for administering the compound in connection with treating malaria; and (c) instructions for treating malaria.

In a further aspect, the kit comprises the antimalarial agent. Examples of antimalarial agents include, but are not limited to, chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, artemisinin derivatives (including dihy Srdroaremisinin (DHA), artesunate, artmether, arteether), artemisinin-based combination therapies (ACT), such as DHA-piperaquine and DHA-piperaquine mefloquine-artesunate.

In a further aspect, the compound and the agent are co-formulated. In a further aspect, the compound and the agent are co-packaged.

In a further aspect, the kit further comprises a plurality of dosage forms, the plurality comprising one or more doses; wherein each dose comprises an effective amount of the compound and the antimalarial agent. In a still further aspect, the effective amount is a therapeutically effective amount. In yet a further aspect, the effective amount is a prophylactically effective amount. In an even further aspect, each dose of the compound and the antimalarial agent are co-formulated. In a still further aspect, each dose of the compound and the antimalarial agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Chemistry Experimentals

Unless otherwise stated all chemicals and reagents were from Sigma-Aldrich Chemical Company in St. Louis, MO (USA), Combi-Blocks, San Diego (CA), or TCI America, Portland (OR) and were used as received. Analytical TLC utilized Merck 60E-254 250 micron precoated silica gel plates and spots were visualized under 254 nm UV light. Flash chromatography over silica gel and C-18 columns were performed using a Selekt One flash chromatography system from Biotage, Uppsala, Sweden. 1H-NMR spectra were obtained using a Bruker 400 MHz Avance NEO NanoBay NMR spectrometer operating at 400.14 MHz. The NMR raw data were analyzed using the NMR Spectrum Analyst software. 1H chemical shifts are reported in parts per million (ppm) relative to internal tetramethylsilane (TMS) standard or residual solvent peak. Coupling constant values (J) are reported in hertz (Hz). HPLC analyses were performed using an Agilent 1260 Infinity instrument with detection at 254 nm and a Phenomenex, Luna® 5 μm C8(2) 100 Å reverse phase LC column 150×4.6 mm at 40° C., and eluted with a gradient range of A/B at 25%: 75% to A/B at 5%: 95% (A:0.05% formic acid in milliQ water, B: 0.05% formic acid in methanol) to a final concentration of A/B at 90%:10%. All ADC derivatives were at least >95% pure for in vitro and in vivo testing as determined by reverse phase HPLC.

### a. Methods for Chemical Synthesis of ADC Derivatives

(i) Step 1

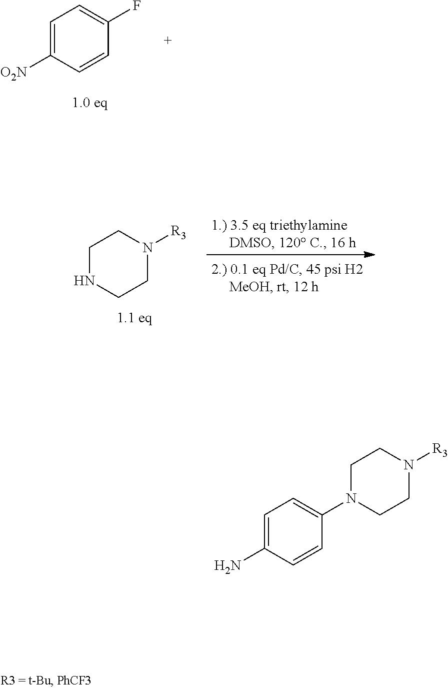

4-Fluoronitrobenzene (1.0 eq) and piperazine (1.1 eq) are dissolved in dimethyl sulfoxide [1.0 M] with trimethylamine (3.5 eq). Reaction mixture is heated for 16 hours at 120° C. to afford piperazine substituted nitrobenzene. Step 2) Substituted nitrobenzene is dissolved in methanol [0.035 M] with Pd/C (0.1 eq) and reduced using a Parr Hydrogenator, reaction ran at room temperature for 12 hours to afford piperazine substituted aniline. Step 3, A) Quinoline (1.0 eq) and substituted aniline (1.0 eq) and phenol (2.0 eq) added to microwave reactor vessel and dissolved in THF [1.0 M]. Reaction heated to 150° C. for 1 hour at high adsorption to afford desired 4-aminoquinoline. B) Quinoline (1.1 eq) and substituted aniline (1.0 eq) dissolved in ethanol (0.2 M). Catalytic amount of fuming HCl was added to reaction mixture and then heated to reflux for 16 hours to afford desired 4-aminoquinoline. C) Quinoline (1.0 eq) and substituted aniline (1.0 eq) and added to microwave reactor vessel and dissolved in THF [1.0 M]. Reaction heated to 120° C. for 20 minutes at high adsorption to afford desired 4-aminoquinoline. Step 4) Boc-protected 4-aminoquinoline (1.0 eq) dissolved in methylene chloride [1.0 M] followed by the addition of trifluoroacetic acid (20 eq) and stirred at room temperature for 2 hours to afford desired deprotected 4-aminoquinoline. Step 5) Deprotected 4-aminoquinoline (1.0 eq) dissolved in dimethylformamide (0.2 M). N,N-diisopropylethylamine (2.0 eq) and iodoethane (1.1 eq) added and reaction stirred at room temp for 16 hours to afford desired alkylated 4-aminoquinoline.

(ii) General Procedure A

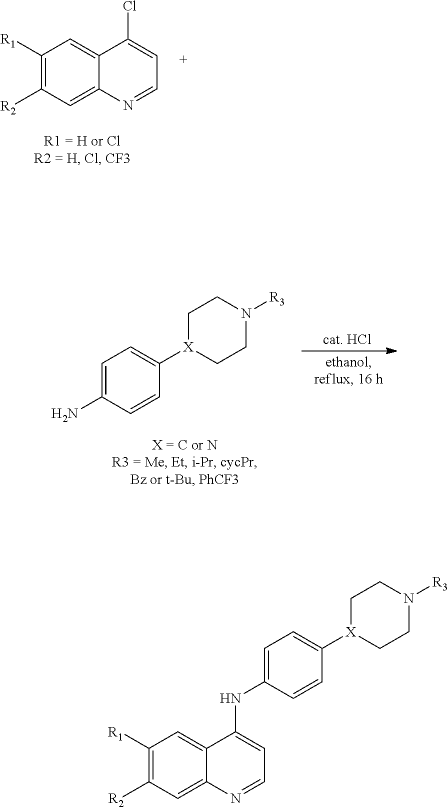

A mixture of aniline (1.1 equiv.) and 4-chloroquinoline (1 equiv.) was dissolved in ethanol (0.1 M) and catalytic fuming HCl (0.05 equiv.) added. The resulting mixture was refluxed for 16 h, where upon the resulting 4-analinoquinoline precipitates as an HCl salt. The reaction mixture was condensed in vacuo and the resulting 4-analinoquinoline was resuspended in methylene chloride and aqueous 2 M sodium hydroxide. The resulting organic layer was extracted with water, brine, dried with magnesium sulfate, concentrated in vacuo, and purified by flash chromatography.

(iii) General Procedure B

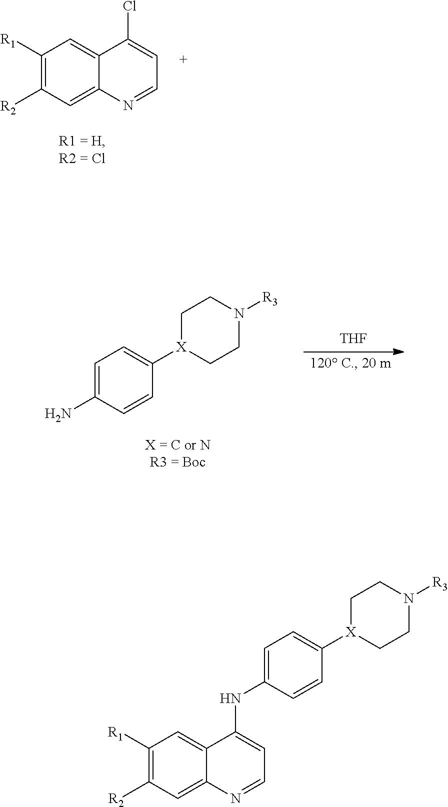

A mixture of aniline (1 equiv.) and 4-chloroquinoline (1 equiv.) dissolved in tetrahydrofuran (1.0 M) in a microwave reactor flask. Flask was sealed and heated to 120° C. for 20 m at high adsorption. Resulting solid was filtered over vacuum filtration with excess THF and air dried. The resulting 4-analinoquinoline was re-suspended in methylene chloride and aqueous 2 M sodium hydroxide and was extracted with water, brine, dried with magnesium sulfate, and concentrated in vacuo. Resulting solid triturated with methanol and air dried over vacuum filtration to afford desired Boc-protected 4-analinoquinoline.

(iv) General Procedure C

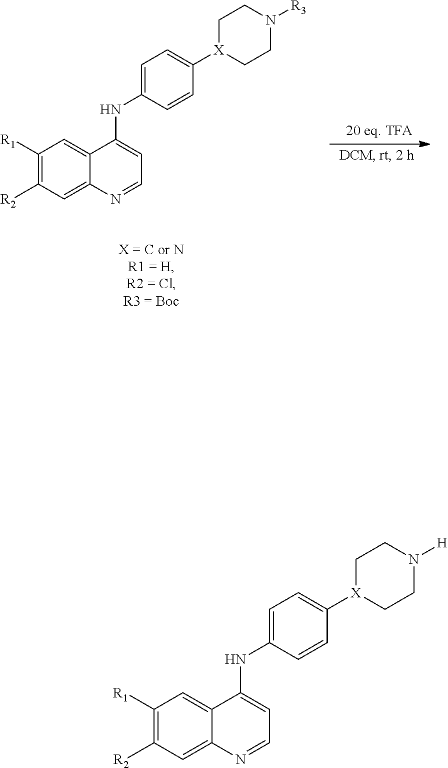

Boc-protected 4-analinoquinoline (1 equiv.) was dissolved in methylene chloride (1.0 M) and trifluoroacetic acid added (20 equiv.), reaction stirred at room temperature for 2 hours. Reaction mixture diluted in methylene chloride and aqueous 2 M sodium hydroxide. Resulting precipitate filtered over vacuum filtration and washed with additional methylene chloride and water. Solid air dried to yield desired 4-analinoquinoline, no further purification needed.

(V) General Procedure D

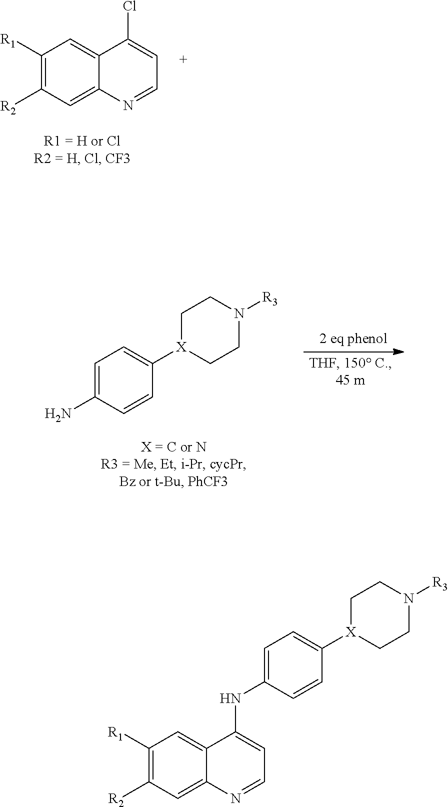

A mixture of aniline (1 equiv.), 4-chloroquinoline (1.2 equiv.) and phenol (2.0 equiv.) dissolved in tetrahydrofuran (1.0 M) in a microwave reactor flask. Flask was sealed and heated to 150° C. for 2 h at high adsorption. Reaction mixture was condensed in vacuo and resuspended in methylene chloride and aqueous 2 M sodium hydroxide. Resulting organic layer was extracted with water, brine, dried with magnesium sulfate, concentrated in vacuo, and purified via flash chromatography to afford desired 4-analinoquinoline.

(vi) General Procedure E

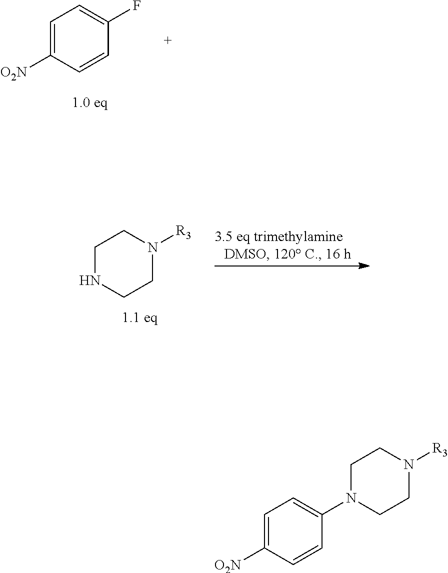

A stirred solution of 4-fluoronitrobenzene (1 equiv.), substituted piperazine (1.1 equiv.), and trimethylamine (3.5 equiv.) in dimethyl sulfoxide (1.0 M) was heated at 120° C. for 16 hours. Reaction mixture allowed to cool to room temperature and resulting solid was diluted in minimum amount of dimethyl sulfoxide and filtered over vacuum filtration. Resulting solid recrystallized using ethanol and ethyl acetate, filtered over vacuum filtration, and air dried to afford piperazine substituted nitrobenzene.

(vii) General Procedure F

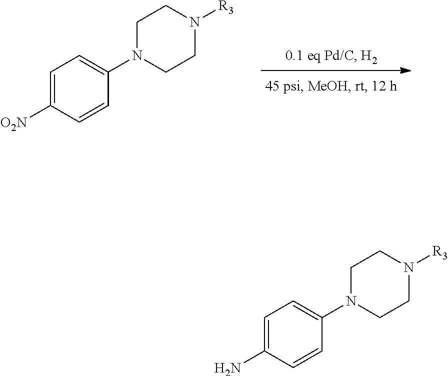

A mixture of piperazine substituted nitrobenzene (1.0 equiv.) and wet palladium on carbon (0.1 equiv.) dissolved in methanol (0.025 M) in a shaker flask. Shaker flask sparged with hydrogen at 45 psi and shaken for 12 hours in Parr Hydrogenator. Pd/C filtered over Celite with excess methanol and resulting filtrate condensed in vacuo. Solid dissolved in ethyl acetate and 2M sodium hydroxide and organic layer extracted with water and brine, dried with magnesium sulfate, condensed in vacuo, and purified via flash chromatography to afford piperazine substituted aniline.

(viii) General Procedure G

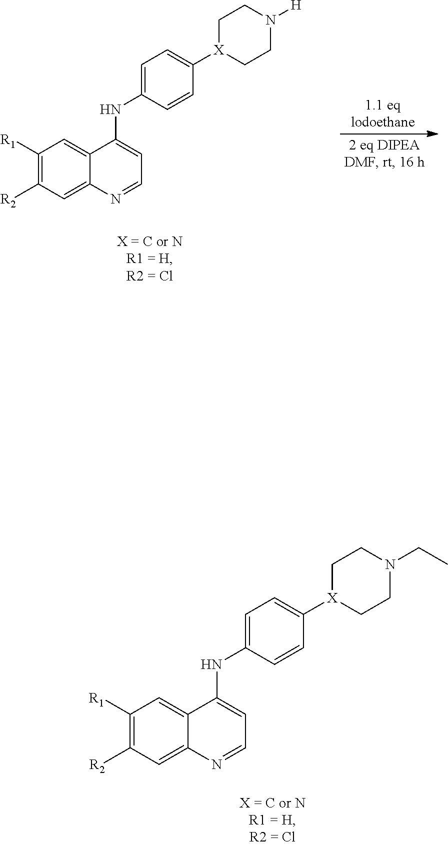

A solution of 4-analinoquinoline (1.0 equiv.), iodoethane (1.1 equiv.), and N,N-diisopropylethylamine (2.0 equiv.) in dimethylformamide (0.2 M) was stirred at room temperature for 16 hr. Upon completion, reaction mixture poured over ice and solid separated by vacuum filtration. Resulting solid dissolved in methylene chloride, dried with magnesium sulfate, condensed in vacuo, and purified using flash chromatography to afford alkylated 4-analinoquinoline.

(ix) General Procedure H

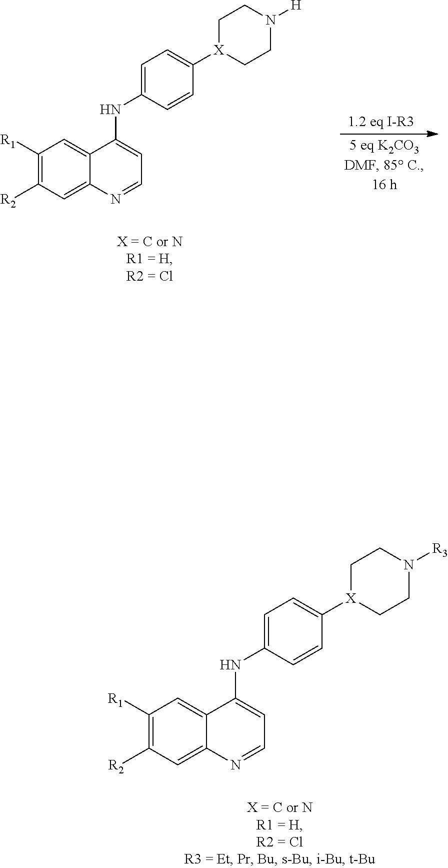

A stirred solution of 4-analinoquinoline (1.0 equiv.), iodo-alkane (1.2 equiv.), and potassium carbonate (5.0 equiv.) in dimethylformamide (0.2 M) was heated to 85° C. for 16 hr. Upon completion, reaction mixture condensed in vacuo. Resulting solid dissolved in methylene chloride and filtered using vacuum filtration. Filtrate extracted with 2 M sodium hydroxide and brine, condensed in vacuo, dry loaded onto Celite and purified via flash chromatography. Fractions containing desired product were condensed in vacuo, and resulting solid upon the addition of aqueous 2M sodium hydroxide was filtered via vacuum filtration and dried in a vacuum oven to afford alkylated 4-analinoquinoline.

### b. Example No. 1-N-(4-(4-benzylpiperazin-1-yl)phenyl)-7-chloroquinolin-4-amine (ADC-008)

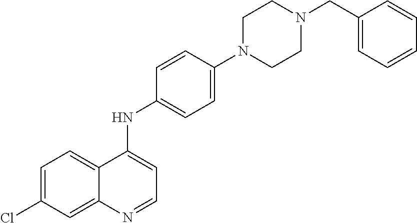 [SMILES: Clc1ccc2c(Nc3ccc(N4CCN(Cc5ccccc5)CC4)cc3)ccnc2c1]

The title compound was prepared with commercially available 4,7-dichloroquinoline (0.198 g, 1.00 mmol) and 4-(4-Benzylpiperazino)aniline (0.296 g, 1.10 mmol) in 10 mL of EtOH using General Procedure A. Title compound purified via reverse phase chromatography (5% methanol/95% water/0.1% TFA). Yield: 54%. HRMS (ESI)—m/z of [C₂₆H₂₆ClN₄⁺]: 429.1841, actual: 429.1834. ¹H-NMR (400 MHz; DMSO-d₆): δ 8.19 (d, J=5.5 Hz, 1H), 7.71 (d, J=2.2 Hz, 1H), 7.38 (dd, J=9.0, 2.2 Hz, 1H), 7.35 (t, J=4.3 Hz, 4H), 7.35 (d, J=4.3 Hz, 4H), 7.30-7.25 (m, 1H), 7.10 (d, J=8.9 Hz, 2H), 6.96 (d, J=9.0 Hz, 2H), 6.48 (d, J=5.5 Hz, 1H), 3.54 (s, 2H), 3.13 (t, J=4.9 Hz, 4H), 2.54 (t, J=4.9 Hz, 4H).

### c. Example No. 2-7-chloro-N-(4-(4-ethylpiperazin-1-yl)phenyl)quinolin-4-amine (ADC-009)

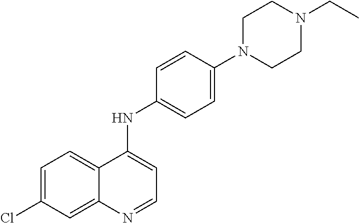 [SMILES: CCN1CCN(c2ccc(Nc3ccnc4cc(Cl)ccc34)cc2)CC1]

The title compound was prepared with commercially available 4,7-dichloroquinoline (0.333 g, 1.68 mmol) and 4-(4-ethylpiperazine-1-yl)aniline (0.309 g, 1.50 mmol) in 10 mL of EtOH using General Procedure A. Title compound purified via normal phase flash chromatography (30% 9:1 Ethyl acetate/trimethylamine in hexanes). Yield: 42%. HRMS (ESI)—m/z of [C₂₁H₂₄ClN₄⁺]: 367.1689, actual: 367.1687. ¹H-NMR (400 MHz; CDCl₃): δ 8.48 (d, J=5.3 Hz, 1H), 8.00 (d, J=2.1 Hz, 1H), 7.83 (d, J=8.9 Hz, 1H), 7.43 (dd, J=8.9, 2.2 Hz, 1H), 7.22-7.18 (m, 2H), 7.01-6.97 (m, 2H), 6.70 (d, J=5.3 Hz, 1H), 6.50 (s, 1H), 3.26 (t, J=5.1 Hz, 4H), 2.64 (t, J=5.0 Hz, 4H), 2.50 (q, J=7.2 Hz, 2H), 1.15 (t, J=7.2 Hz, 3H).

### d. Example No. 3-7-chloro-N-(4-(4-methylpiperazin-1-yl)phenyl)quinolin-4-amine (ADC-010)

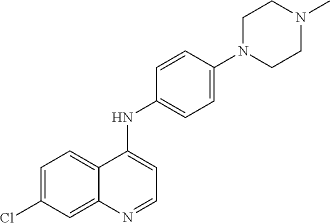 [SMILES: CN1CCN(c2ccc(Nc3ccnc4cc(Cl)ccc34)cc2)CC1]

The title compound was prepared with commercially available 4,7-dichloroquinoline (0.328 g, 1.66 mmol) and 4-(4-methylpiperazinol)aniline (0.287 g, 1.50 mmol) in 10 mL of EtOH using General Procedure A. Title compound purified via normal phase flash chromatography (30% 9:1 Ethyl acetate/trimethylamine in hexanes). Yield: 57%. HRMS (ESI)—m/z of [C₂₄H₂₂ClN₄⁺]: 353.1533, actual: 353.1526. ¹H-NMR (400 MHz; CDCl₃): δ 8.50 (d, J=5.3 Hz, 1H), 8.03 (d, J=2.1 Hz, 1H), 7.86 (d, J=9.0 Hz, 1H), 7.45 (dd, J=8.9, 2.2 Hz, 1H), 7.24-7.20 (m, 2H), 7.03-6.99 (m, 2H), 6.72 (d, J=5.3 Hz, 1H), 6.55 (s, 1H), 3.27 (t, J=5.0 Hz, 4H), 2.63 (t, J=5.0 Hz, 4H), 2.40 (s, 3H).

### e. Example No. 4-tert-butyl 4-(4-((7-chloroquinolin-4-yl)amino)phenyl)piperazine-1-carboxylate

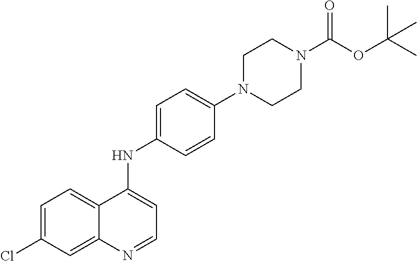 [SMILES: CC(C)(C)OC(=O)N1CCN(c2ccc(Nc3ccnc4cc(Cl)ccc34)cc2)CC1]

The title compound was prepared with commercially available 4,7-dichloroquinoline (1.096 g, 5.53 mmol) and 1-boc-4-(4-aminophenyl)piperazine (1.531 g, 5.52 mmol) in 10 mL of THF using General Procedure B. Yield: 69%. ¹H-NMR (400 MHz; CDCl₃): δ 8.51 (d, J=5.3 Hz, 1H), 8.03 (d, J=2.1 Hz, 1H), 7.87 (d, J=9.0 Hz, 1H), 7.46 (dd, J=9.0, 2.2 Hz, 1H), 7.25-7.22 (m, 2H), 7.03-6.99 (m, 2H), 6.73 (d, J=5.3 Hz, 1H), 6.57 (s, 1H), 3.63 (t, J=5.2 Hz, 4H), 3.18 (t, J=5.1 Hz, 4H), 1.52 (s, 9H).

### f. Example No. 5-7-chloro-N-(4-(piperazin-1-yl)phenyl)quinolin-4-amine (ADC-011)

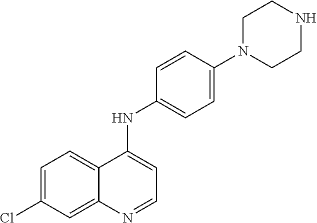 [SMILES: Clc1ccc2c(Nc3ccc(N4CCNCC4)cc3)ccnc2c1]

The title compound was prepared with tert-butyl 4-(4-((7-chloroquinolin-4-yl)amino)phenyl)piperazine-1-carboxylate (1.501 g, 3.42 mmol) and TFA (4 mL, 52.3 mmol) in 5 mL of DCM using General Procedure B. Yield: 0.040 g, 40%. HRMS (ESI)—m/z of [C₁₉H₂₀ClN₄⁺]: 339.1376, actual: 339.1372. ¹H-NMR (MHz; CDCl₃): δ 8.54 (d, J=5.3 Hz, 1H), 8.03 (d, J=2.1 Hz, 1H), 7.85 (d, J=9.0 Hz, 1H), 7.46 (dd, J=9.0, 2.2 Hz, 1H), 7.30-7.27 (m, 2H), 7.25-7.22 (m, 2H), 6.91 (d, J=5.3 Hz, 1H), 6.55 (dd, J=2.6, 0.3 Hz, 1H), 3.24-3.20 (m, 2H), 2.80-2.74 (m, 2H), 2.66-2.63 (m, 1H), 1.89-1.84 (m, 2H), 1.72-1.62 (m, 2H).

### g. Example No. 6-7-chloro-N-(4-(4-cyclopropylpiperazin-1-yl)phenyl)quinolin-4-amine (ADC-013)

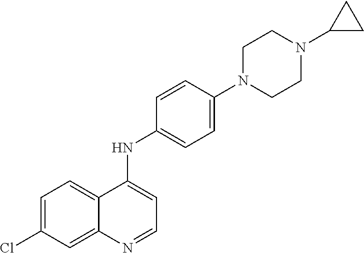 [SMILES: Clc1ccc2c(Nc3ccc(N4CCN(C5CC5)CC4)cc3)ccnc2c1]

The title compound was prepared with commercially available 4,7-dichloroquinoline (0.154 g, 0.778 mmol) and 4-(4-cyclopropylpiperazin-1-yl)aniline (0.287 g, 1.50 mmol) in 3 mL of EtOH using General Procedure A. Title compound purified via normal phase flash chromatography (30% 9:1 Ethyl acetate/trimethylamine in hexanes). Yield: 0.019 g, 6%. HRMS (ESI)—m/z of [C₂₂H₂₄ClN₄⁺]: 379.1690, actual: 379.1685. ¹H-NMR (400 MHz; DMSO-d₆): δ 8.91 (s, 1H), 8.41 (d, J=9.1 Hz, 1H), 8.37 (d, J=5.4 Hz, 1H), 7.85 (d, J=2.2 Hz, 1H), 7.53 (dd, J=9.0, 2.3 Hz, 1H), 7.22-7.18 (m, 2H), 7.03-6.99 (m, 2H), 6.63 (d, J=5.4 Hz, 1H), 3.11 (t, J=5.0 Hz, 4H), 2.69 (t, J=5.0 Hz, 4H), 1.67 (tt, J=6.7, 3.4 Hz, 1H), 0.48-0.43 (m, 2H), 0.37-0.34 (m, 2H).

### h. Example No. 7-6-chloro-N-(4-(4-ethylpiperazin-1-yl)phenyl)quinolin-4-amine (ADC-014)

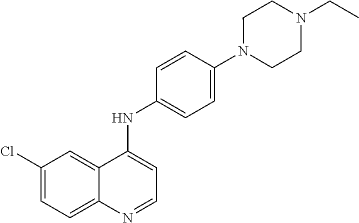 [SMILES: CCN1CCN(c2ccc(Nc3ccnc4ccc(Cl)cc34)cc2)CC1]

The title compound was prepared with commercially available 4,6-dichloroquinoline (0.261 g, 1.32 mmol), 4-(4-ethylpiperazine-1-yl)aniline (0.208 g, 1.01 mmol), and phenol (0.190 g, 2.01 mmol) in 1 mL of THF using General Procedure D. Title compound purified via normal phase flash chromatography (0.5% methanol in methylene chloride). Yield: 0.232 g, 62%. HRMS (ESI)—m/z of [C₂₁H₂₄ClN₄⁺]: 367.1689, actual: 367.1686. ¹H-NMR (400 MHz; CDCl₃): δ 8.51 (dd, J=5.3, 0.4 Hz, 1H), 7.98 (d, J=9.0 Hz, 1H), 7.91 (d, J=2.2 Hz, 1H), 7.63 (dd, J=9.0, 2.2 Hz, 1H), 7.24-7.20 (m, 2H), 7.04-7.00 (m, 2H), 6.75 (d, J=5.3 Hz, 1H), 6.43 (d, J=0.2 Hz, 1H), 3.28 (t, J=5.1 Hz, 4H), 2.67 (t, J=5.1 Hz, 4H), 2.52 (q, J=7.2 Hz, 2H), 1.17 (t, J=7.2 Hz, 3H).

### i. Example No. 8-tert-butyl 4-(4-((7-chloroquinolin-4-yl)amino)phenyl)piperidine-1-carboxylate

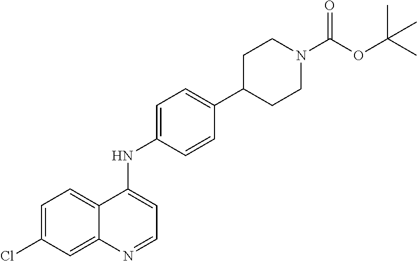 [SMILES: CC(C)(C)OC(=O)N1CCC(c2ccc(Nc3ccnc4cc(Cl)ccc34)cc2)CC1]

The title compound was prepared with commercially available 4,7-dichloroquinoline (1.115 g, 5.81 mmol) and tert-butyl 4-(4-aminophenyl)piperidine-1-carboxylate (1.603 g, 5.80 mmol) in 10 mL of THF using General Procedure B. Yield: 87%. ¹H-NMR (400 MHz; DMSO-d₆): δ 9.04 (s, 1H), 8.45-8.41 (m, 2H), 7.89 (d, J=2.2 Hz, 1H), 7.57 (dd, J=9.0, 2.3 Hz, 1H), 7.33-7.28 (m, 4H), 6.87 (d, J=5.4 Hz, 1H), 4.09 (d, J=11.7 Hz, 2H), 2.83-2.67 (m, 3H), 1.79 (d, J=13.2 Hz, 2H), 1.56-1.45 (m, 2H), 1.43 (s, 9H).

### j. Example No. 9-7-chloro-N-(4-(piperidin-4-yl)phenyl)quinolin-4-amine (ADC-015)

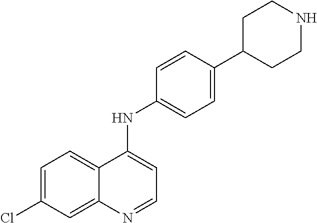 [SMILES: Clc1ccc2c(Nc3ccc(C4CCNCC4)cc3)ccnc2c1]

The title compound was prepared with tert-butyl 4-(4-((7-chloroquinolin-4-yl)amino)phenyl)piperidine-1-carboxylate (1.536 g, 3.51 mmol) and TFA (30 equiv., 8.03 mL 105 mmol) in 10 mL of DCM using General Procedure B. Yield: 69%. ¹H-NMR (400 MHz; CDCl₃): δ 8.54 (d, J=5.3 Hz, 1H), 8.03 (d, J=2.1 Hz, 1H), 7.85 (d, J=9.0 Hz, 1H), 7.46 (dd, J=9.0, 2.2 Hz, 1H), 7.30-7.27 (m, 2H), 7.25-7.22 (m, 2H), 6.91 (d, J=5.3 Hz, 1H), 6.55 (dd, J=2.6, 0.3 Hz, 1H), 3.24-3.20 (m, 2H), 2.80-2.74 (m, 2H), 2.66-2.63 (m, 1H), 1.89-1.84 (m, 2H), 1.72-1.62 (m, 2H).

### (i) 1-(tert-butyl)-4-(4-nitrophenyl)piperazine

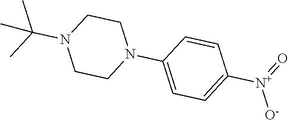 [SMILES: CC(C)(C)N1CCN(c2ccc([N+](=O)[O-])cc2)CC1]

Intermediate was prepared from commercially available 4-fluoronitrobenzene (0.78 g, 5.5 mmol), tert-butyl piperazine (0.85 g, 6.0 mmol), and trimethylamine (1.82 g, 18.0 mmol) in 5 mL DMSO using General Procedure E. Yield: 53%.

### (ii) 4-(4-(tert-butyl)piperazin-1-yl)aniline

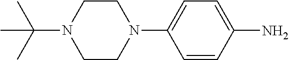 [SMILES: CC(C)(C)N1CCN(c2ccc(N)cc2)CC1]

Intermediate was prepared from 1-(tert-butyl)-4-(4-nitrophenyl)piperazine (0.694 g, 2.62 mmol) and wet 10% Pd/C (0.041 g, 0.38 mmol) in 100 mL MeOH using General Procedure F. Product purified via flash chromatography (1% methanol in methylene chloride, isocratic over basic alumina) Product isolated as brown solid Yield: 0.340 g, 56%. ¹H-NMR (400 MHz; CDCl₃): δ 6.82 (d, J=8.8 Hz, 2H), 6.65 (d, J=8.8 Hz, 2H), 3.41 (s, 1H), 3.06 (t, J=4.9 Hz, 4H), 2.74 (t, J=4.9 Hz, 4H), 1.11 (s, 9H).

### k. Example No. 10-N-(4-(4-(tert-butyl)piperazin-1-yl)phenyl)-7-chloroquinolin-4-amine (ADC-017)

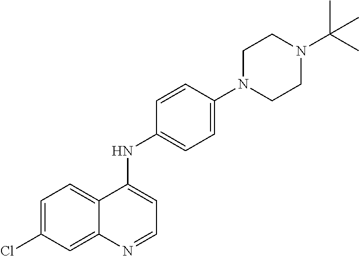 [SMILES: CC(C)(C)N1CCN(c2ccc(Nc3ccnc4cc(Cl)ccc34)cc2)CC1]

The title compound was prepared with 4-(4-(tert-butyl)piperazin-1-yl)aniline (0.205 g, 0.878 mmol), and commercially available 4,7-dichloroquinoline (0.201 g, 1.01 mmol) and phenol (0.195 g, 2.07 mmol) in 1 mL of THF using General Procedure D. Compound purified by reverse phase flash chromatography (5% to 90% methanol in water with 0.1% TFA). Title compound isolated as a tan solid, yield: 0.072 g, 18%. HRMS (ESI)—m/z of [⁺C₂₃H₂₈ClN₄]: 395.2003, actual: 395.2000. ¹H-NMR (400 MHz; CDCl₃): δ 8.51 (d, J=5.3 Hz, 1H), 8.03 (d, J=2.1 Hz, 1H), 7.85 (d, J=9.0 Hz, 1H), 7.46 (dd, J=8.9, 2.2 Hz, 1H), 7.23-7.20 (m, 2H), 7.03-6.99 (m, 2H), 6.72 (d, J=5.3 Hz, 1H), 6.52 (d, J=0.2 Hz, 1H), 3.26 (t, J=4.9 Hz, 4H), 2.80 (t, J=4.9 Hz, 4H), 1.16 (s, 9H).

### (i) 1-(4-nitrophenyl)-4-(4-(trifluoromethyl)phenyl)piperazine

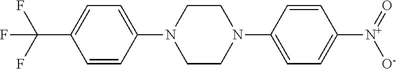 [SMILES: O=[N+]([O-])c1ccc(N2CCN(c3ccc(C(F)(F)F)cc3)CC2)cc1]

Intermediate was prepared from commercially available 4-fluoronitrobenzene (2.542 g, 18.0 mmol), 1-(4-trifluoromethylphenyl)piperazine (4.567 g, 19.8 mmol), and trimethylamine (8.5 mL, 61 mmol) in 15 mL DMSO using General Procedure E. Product isolated as orange solid, yield: 1.254 g, 20%. ¹H-NMR (400 MHz, CDCl₃) 1H-NMR (400 MHz; CDCl₃): δ 8.21-8.17 (m, 2H), 7.55 (d, J=8.6 Hz, 2H), 6.97 (d, J=8.7 Hz, 2H), 6.91-6.87 (m, 2H), 3.64 (dd, J=6.5, 4.1 Hz, 4H), 3.51 (dd, J=6.5, 4.1 Hz, 4H).

### (ii) 4-(4-(4-(trifluoromethyl)phenyl)piperazin-1-yl)aniline

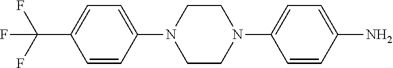 [SMILES: Nc1ccc(N2CCN(c3ccc(C(F)(F)F)cc3)CC2)cc1]

Intermediate was prepared from 1-(4-nitrophenyl)-4-(4-(trifluoromethyl)phenyl)piperazine (1.254 g, 3.57 mmol) and wet 10% Pd/C (0.145 g, 0.38 mmol) in 100 mL MeOH using general procedure F. Product isolated as a straw colored solid, yield: 0.892 g, 78%. ¹H-NMR (400 MHz; CDCl₃): δ 7.54-7.51 (m, 2H), 7.02-6.98 (m, 2H), 6.90-6.87 (m, 2H), 6.72-6.69 (m, 2H), 3.50 (s, 1H), 3.46-3.43 (m, 4H), 3.22-3.19 (m, 4H).

### l. Example No. 11-7-chloro-N-(4-(4-(4-(trifluoromethyl)phenyl)piperazin-1-yl)phenyl)quinolin-4-amine (ADC-018)

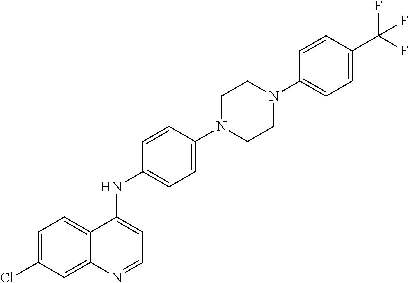 [SMILES: FC(F)(F)c1ccc(N2CCN(c3ccc(Nc4ccnc5cc(Cl)ccc45)cc3)CC2)cc1]

The title compound was prepared with 4-(4-(4-(trifluoromethyl)phenyl) piperazin-1-yl) aniline (0.205 g, 0.878 mmol), and commercially available 4,7-dichloroquinoline (0.126 g, 0.64 mmol) and phenol (0.119 g, 1.28 mmol) in 0.8 mL of THF using General Procedure D. Product isolated as yellow solid, yield: 0.141 g, 46%. HRMS (ESI)—m/z of [C₂₆H₂₃ClF₃N₄⁺]: 483.1564, actual: 483.1562. ¹H-NMR (400 MHz; CDCl₃): δ 8.52 (d, J=5.3 Hz, 1H), 8.04 (d, J=2.1 Hz, 1H), 7.87 (d, J=9.0 Hz, 1H), 7.55 (d, J=8.6 Hz, 2H), 7.47 (dd, J=8.9, 2.2 Hz, 1H), 7.25 (s, 3H), 7.08-7.04 (m, 2H), 7.02 (d, J=8.7 Hz, 2H), 6.75 (d, J=5.3 Hz, 1H), 6.56 (s, 1H), 3.51-3.48 (m, 4H).

### M. Example No. 12-7-chloro-N-(4-(1-ethylpiperidin-4-yl)phenyl)quinolin-4-amine (ADC-020)

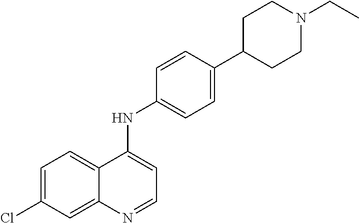 [SMILES: CCN1CCC(c2ccc(Nc3ccnc4cc(Cl)ccc34)cc2)CC1]

The title compound was prepared from 7-chloro-N-(4-(piperidin-4-yl)phenyl)quinolin-4-amine (ADC-015) (0.338 g, 1.00 mmol) and commercially available iodoethane (0.09 mL, 1.12 mmol), N,N-diisopropylethylamine (0.35 mL, 2.00 mmol) in 5 mL of DMF using General Procedure G. Product isolated as of white powder, yield: 0.108 g, 30%. HRMS (ESI)—m/z of [C₂₂H₂₅ClN₃⁺]: 366.1737, actual: 366.1732. ¹H-NMR (400 MHz; DMSO-d₆): δ 9.02 (s, 1H), 8.44-8.41 (m, 2H), 7.88 (d, J=2.2 Hz, 1H), 7.56 (dd, J=9.0, 2.3 Hz, 1H), 7.29 (d, J=2.1 Hz, 4H), 6.85 (d, J=5.3 Hz, 1H), 3.01-2.98 (m, 2H), 2.36 (ddd, J=5.5, 1.2, 0.6 Hz, 2H), 1.97-1.97 (m, 2H), 1.79-1.76 (m, 2H), 1.65 (qd, J=12.3, 3.2 Hz, 2H), 1.03 (t, J=7.2 Hz, 3H).

### n. Example No. 13-7-chloro-N-(4-(1-isopropylpiperidin-4-yl)phenyl)quinolin-4-amine (ADC-021)

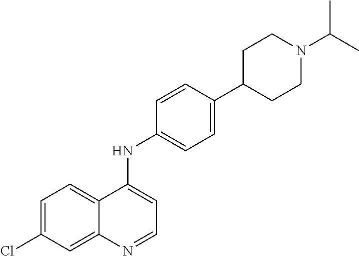 [SMILES: CC(C)N1CCC(c2ccc(Nc3ccnc4cc(Cl)ccc34)cc2)CC1]

The title compound was prepared with commercially available 4,7-dichloroquinoline (0.218 g, 1.00 mmol), 4-(1-isopropyl)piperadin-1-yl)aniline (0.219 g, 1.10 mmol), and fuming HCl (0.1 mL) in 3 mL of EtOH using General Procedure A. Title compound purified via reverse phase flash chromatography (5% to 95% methanol in water with 0.1% TFA) followed by normal phase flash chromatography (5% to 30% ethyl acetate in hexanes). Product isolated as white solid, yield: 0.111 g, 29%. HRMS (ESI)—m/z of [C₂₃H₂₇ClN₃⁺]: 380.1894, actual: 380.1890. ¹H-NMR (400 MHz; MeOD): δ 8.37 (d, J=5.6 Hz, 1H), 8.31 (dd, J=9.0, 0.4 Hz, 1H), 7.87 (d, J=1.9 Hz, 1H), 7.51 (dd, J=9.0, 2.2 Hz, 1H), 7.37-7.32 (m, 4H), 6.88 (d, J=5.6 Hz, 1H), 3.12 (d, J=11.6 Hz, 2H), 2.90-2.84 (m, 1H), 2.64 (tt, J=12.0, 3.9 Hz, 1H), 2.48-2.42 (m, 2H), 1.95 (dd, J=12.8, 1.7 Hz, 2H), 1.89-1.79 (m, 2H), 1.17 (d, J=6.6 Hz, 6H).

### O. Example No. 14-N-(4-(4-ethylpiperazin-1-yl)phenyl)-7-(trifluoromethyl)quinolin-4-amine (ADC-024)

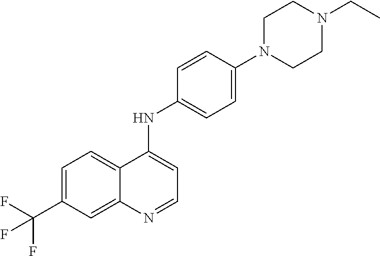 [SMILES: CCN1CCN(c2ccc(Nc3ccnc4cc(C(F)(F)F)ccc34)cc2)CC1]

The title compound was prepared with commercially available 4,7-dichloroquinoline (0.333 g, 1.68 mmol) and 4-(4-ethylpiperazine-1-yl)aniline (0.309 g, 1.50 mmol) in 10 mL of EtOH using General Procedure A. Title compound purified via normal phase flash chromatography (30% 9:1 Ethyl acetate/trimethylamine in hexanes). Yield: 42%. HRMS (ESI)—m/z of [C₂₂H₂₄F₃N₄⁺]: 401.1953, actual: 401.1948. ¹H-NMR (400 MHz; DMSO-d₆): δ 9.05 (s, 1H), 8.64-8.62 (m, 1H), 8.49 (d, J=5.4 Hz, 1H), 8.14 (t, J=0.3 Hz, 1H), 7.78-7.76 (m, 1H), 7.23 (d, J=8.9 Hz, 2H), 7.03 (d, J=9.0 Hz, 2H), 6.74 (d, J=5.4 Hz, 1H), 3.17 (t, J=5.0 Hz, 4H), 2.41-2.36 (m, 2H), 1.05 (t, J=7.2 Hz, 3H).

### P. Example No. 15-7-bromo-N-(4-(4-ethylpiperazin-1-yl)phenyl)quinolin-4-amine (ADC-025)

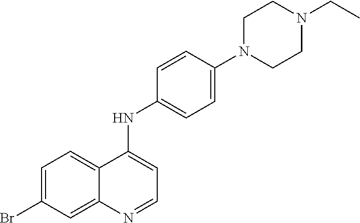 [SMILES: CCN1CCN(c2ccc(Nc3ccnc4cc(Br)ccc34)cc2)CC1]

The title compound was prepared with commercially available 4,7-dichloroquinoline (0.333 g, 1.68 mmol) and 4-(4-ethylpiperazine-1-yl)aniline (0.309 g, 1.50 mmol) in 10 mL of EtOH using General Procedure A. Title compound purified via normal phase flash chromatography (30% 9:1 Ethyl acetate/trimethylamine in hexanes). Yield: 42%. HRMS (ESI)—m/z of [C₂₁H₂₄BrN₄⁺]: 413.1160, actual: 413.1160. ¹H-NMR (400 MHz; CDCl₃): δ 8.48 (d, J=5.3 Hz, 1H), 8.00 (d, J=2.1 Hz, 1H), 7.83 (d, J=9.0 Hz, 1H), 7.43 (dd, J=8.9, 2.2 Hz, 1H), 7.22-7.18 (m, 2H), 7.01-6.97 (m, 2H), 6.69 (d, J=5.3 Hz, 1H), 6.53 (s, 1H), 3.49 (s, 1H), 3.24 (t, J=5.0 Hz, 4H), 2.61 (t, J=5.0 Hz, 4H), 2.37 (s, 3H).

### q. Example No. 16-7-chloro-N-(4-(1-propylpiperidin-4-yl)phenyl)quinolin-4-amine (ADC-026)

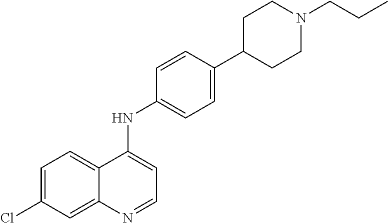 [SMILES: CCCN1CCC(c2ccc(Nc3ccnc4cc(Cl)ccc34)cc2)CC1]

The title compound was prepared from 7-chloro-N-(4-(piperidin-4-yl)phenyl)quinolin-4-amine (ADC-015)(0.338 g, 1.00 mmol) and commercially available iodopropane (0.12 mL, 1.2 mmol), potassium carbonate (0.690, 4.99 mmol) in 5 mL of DMF using General Procedure H. Product purified by reverse phase flash chromatography (5% to 90% methanol in water with 0.1% TFA) and isolated as of white powder, yield: 0.099 g, 26%. HRMS (ESI)—m/z of [C₂₄H₂₉ClN₃⁺]: 394.2051, actual: 394.2046. ¹H-NMR (400 MHz; DMSO-d₆): δ 9.04 (s, 1H), 8.43 (dd, J=7.2, 5.3 Hz, 2H), 7.89 (d, J=2.2 Hz, 1H), 7.57 (dd, J=9.0, 2.3 Hz, 1H), 7.30 (d, J=2.5 Hz, 4H), 6.86 (d, J=5.4 Hz, 1H), 2.97 (d, J=11.3 Hz, 2H), 2.26 (dd, J=8.1, 6.8 Hz, 2H), 2.00-1.94 (m, 2H), 1.79-1.75 (m, 2H), 1.71-1.60 (m, 2H), 1.47 (sextet, J=7.4 Hz, 2H), 0.88 (t, J=7.4 Hz, 3H).

### r. Example No. 17-N-(4-(1-butylpiperidin-4-yl)phenyl)-7-chloroquinolin-4-amine (ADC-027)

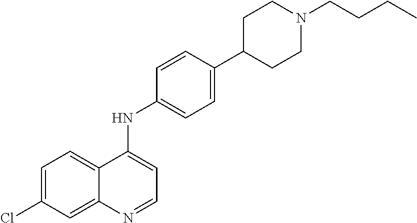 [SMILES: CCCCN1CCC(c2ccc(Nc3ccnc4cc(Cl)ccc34)cc2)CC1]

The title compound was prepared from 7-chloro-N-(4-(piperidin-4-yl)phenyl)quinolin-4-amine (ADC-015) (0.338 g, 1.00 mmol) and commercially available iodobutane (0.14 mL, 1.2 mmol), potassium carbonate (0.696, 5.04 mmol) in 5 mL of DMF using General Procedure H. Product purified by reverse phase flash chromatography (5% to 90% methanol in water with 0.1% TFA) and isolated as of white powder, yield: 0.249 g, 63%. HRMS (ESI)—m/z of [C₂₄H₂₉ClN₃⁺]: 394.2051, actual: 394.2046. ¹H-NMR (400 MHz; DMSO-d₆): δ 9.05 (s, 1H), 8.43 (dd, J=7.2, 5.2 Hz, 2H), 7.89 (d, J=2.2 Hz, 1H), 7.57 (dd, J=9.0, 2.2 Hz, 1H), 7.32-7.27 (m, 4H), 6.86 (d, J=5.4 Hz, 1H), 2.97 (d, J=11.5 Hz, 2H), 2.29 (t, J=7.4 Hz, 2H), 1.96 (td, J=11.6, 2.2 Hz, 2H), 1.77 (dd, J=12.6, 1.6 Hz, 2H), 1.65 (qd, J=12.2, 3.3 Hz, 2H), 1.47-1.40 (m, 2H), 1.31 (dq, J=14.9, 7.4 Hz, 2H), 0.90 (t, J=7.3 Hz, 3H).

### s. Example No. 18-7-chloro-N-(4-(1-isobutylpiperidin-4-yl)phenyl)quinolin-4-amine (ADC-028)

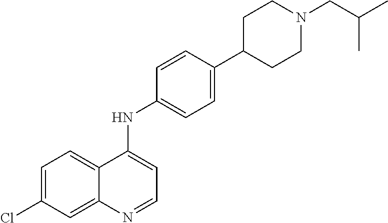 [SMILES: CC(C)CN1CCC(c2ccc(Nc3ccnc4cc(Cl)ccc34)cc2)CC1]

The title compound was prepared from 7-chloro-N-(4-(piperidin-4-yl)phenyl)quinolin-4-amine (0.339 g, 1.00 mmol) and commercially available 1-iodo-2-methylpropane (0.14 mL, 1.2 mmol), potassium carbonate (0.696, 5.04 mmol) in 5 mL of DMF using General Procedure H. Product purified by reverse phase flash chromatography (5% to 90% methanol in water with 0.1% TFA) and isolated as a white solid, yield: 0.074 g, 19%. HRMS (ESI)—m/z of [C₂₄H₂₉ClN₃⁺]: 394.2051, actual: 394.2045. ¹H-NMR (400 MHz; DMSO-d₆): δ 9.05 (t, J=0.2 Hz, 1H), 8.44-8.41 (m, 2H), 7.89 (d, J=2.2 Hz, 1H), 7.57 (dd, J=9.0, 2.3 Hz, 1H), 7.32-7.27 (m, 4H), 6.86 (d, J=5.4 Hz, 1H), 2.93 (d, J=11.4 Hz, 2H), 2.06 (d, J=7.4 Hz, 2H), 1.99-1.93 (m, 2H), 1.81-1.75 (m, 3H), 1.71-1.61 (m, 2H), 0.88 (d, J=6.6 Hz, 6H).

### t. Example No. 19-N-(4-(1-(sec-butyl)piperidin-4-yl)phenyl)-7-chloroquinolin-4-amine (ADC-029)

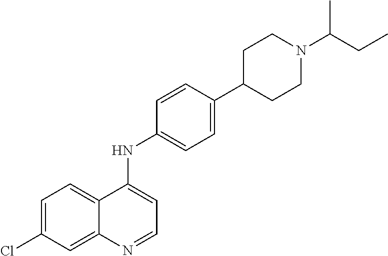 [SMILES: CCC(C)N1CCC(c2ccc(Nc3ccnc4cc(Cl)ccc34)cc2)CC1]

The title compound was prepared from 7-chloro-N-(4-(piperidin-4-yl)phenyl)quinolin-4-amine (ADC-015) (0.342 g, 1.01 mmol) and commercially available 2-iodobutane (0.14 mL, 1.2 mmol), potassium carbonate (0.696, 5.04 mmol) in 5 mL of DMF using General Procedure H. Product purified by reverse phase flash chromatography (5% to 90% methanol in water with 0.1% TFA) and isolated as a white solid, yield: 0.201 g, 50%. HRMS (ESI)—m/z of [C₂₄H₂₉ClN₃⁺]: 394.2051, actual: 394.2046. ¹H-NMR (400 MHz; DMSO-d₆): δ 9.04 (s, 1H), 8.43 (dd, J=7.2, 4.9 Hz, 2H), 7.89 (d, J=2.2 Hz, 1H), 7.57 (dd, J=9.0, 2.3 Hz, 1H), 7.31-7.27 (m, 4H), 6.86 (d, J=5.4 Hz, 1H), 2.80 (d, J=8.1 Hz, 2H), 2.48-2.37 (m, 3H), 2.23-2.18 (m, 1H), 1.78 (d, J=12.9 Hz, 2H), 1.69-1.48 (m, 3H), 1.28 (dt, J=13.5, 7.4 Hz, 1H), 0.94 (d, J=6.6 Hz, 3H), 0.88 (t, J=7.4 Hz, 3H).

### U. Example No. 20-7-chloro-N-(4-(4-isobutylpiperazin-1-yl)phenyl)quinolin-4-amine (ADC-030)

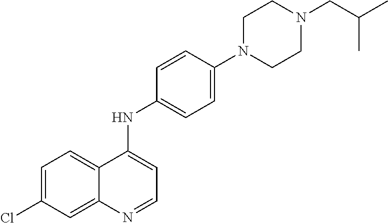 [SMILES: CC(C)CN1CCN(c2ccc(Nc3ccnc4cc(Cl)ccc34)cc2)CC1]

The title compound was prepared from 7-chloro-N-(4-(piperazin-1-yl)phenyl)quinolin-4-amine (ADC-011) (0.169 g, 0.50 mmol) and commercially available 1-iodo-2-methylpropane (0.12 mL, 1.2 mmol), potassium carbonate (0.690, 4.99 mmol) in 5 mL of DMF using General Procedure H. Product purified by reverse phase flash chromatography (5% to 90% methanol in water with 0.1% TFA) and isolated as yellow powder. Yield: 0.037 g, 19%. HRMS (ESI)—m/z of [C₂₃H28ClN₄⁺]: 395.2003, actual: 395.1992. ¹H-NMR (400 MHz; CDCl₃): δ 8.51 (d, J=5.3 Hz, 1H), 8.03 (d, J=2.1 Hz, 1H), 7.85 (d, J=8.9 Hz, 1H), 7.46 (dd, J=8.9, 2.2 Hz, 1H), 7.23-7.20 (m, 2H), 7.03-6.99 (m, 2H), 6.72 (d, J=5.3 Hz, 1H), 6.52 (s, 1H), 3.25 (t, J=5.1 Hz, 4H), 2.60 (t, J=5.0 Hz, 4H), 2.18 (d, J=7.4 Hz, 2H), 1.91-1.81 (m, 1H), 0.96 (d, J=6.6 Hz, 6H).

2. Biology Experimentals

a. Plasmodium falciparum Isolation and Culture

Laboratory strains of P. falciparum were cultured in human erythrocytes by standard methods (Trager, W.; Jensen, J. B. Human malaria parasites in continuous culture. Science 1976, 193 (4254), 673-675. DOI: 10.1126/science.781840 from NLM Medline). The parasites were grown in culture medium with fresh human erythrocytes maintained at 2% hematocrit at 37° C. in low-oxygen conditions (5% 02, 5% CO₂, 90% N₂). The culture medium used was RPMI-1640 with 25 mg/L gentamicin sulfate, 45 mg/L Albumax II, 10 mM glucose, and 25 mM HEPES buffer (Smilkstein et al., Simple and inexpensive fluorescence-based technique for high-throughput antimalarial drug screening. Antimicrob Agents Chemother 2004, 48 (5), 1803-1806. DOI: 10.1128/aac.48.5.1803-1806.2004). Cultures were maintained at less than 10% parasitemia by transfer of infected cells to fresh erythrocytes and culture medium every 3 or 4 days.

b. In Vitro Activity

ADC compounds antiplasmodial activity (IC₅₀), against cultured chloroquine resistant (D6) and multi-drug resistant (Dd2) P. falciparum parasites using fluorescence-based assay. In vivo efficiency: ADCs antiplasmodial efficiency (ED₅₀, ED₉₀) assessed in murine malaria model against P. yoelii (Py). Metabolic stability: ADCs were assessed for metabolic stability (t_{1/2}, Clᵢₙₜ) in pooled murine liver microsomes. Referring to Table 1, ND=not detected.

C. In Vivo Activity

Selected ADC derivatives were also tested in vivo in mice using the standard 4-day test protocol as described previously. For this work the rodent malaria parasite Plasmodium yoelii was used and the mice were CF1 female. Briefly, animals were inoculated with 35,000 parasitized red blood cells (obtained from a donor animal) on Day 0 of the experiment. Beginning on the next day (Day 1) and again on the 3 succeeding days (Days 2, 3, and 4) the animals received oral doses of the test agent dissolved in the dosing vehicle PEG-400. On Day 5 a blood sample was taken from the tail vein and examined microscopically for the presence of parasites using Giemsa stained and methanol fixed blood smears. The cited ED₅₀ values represent the drug dose required to reduce percent parasitemia by 50% relative to the untreated control group. The typical % parasitemia in control mice under the conditions of this protocol is roughly 20%. Notice that all of the tested ADC derivatives were active and effective in this assay in which chloroquine yields and ED₅₀ value of 1.5 mg/kg/day. ED₅₀ values for the ADC derivatives ranged from 1.1 to 3.4 mg/kg/day with ADC-021 exhibiting the most potent efficacy at 1.1 mg/kg/day. Taken together, our studies demonstrate that Amodiachin derivatives are active against multidrug resistant P. falciparum malaria parasites in vitro and highly effective in vivo against malaria in a mouse model of the disease.

d. In Vitro Antiplasmodial Testing and IC₅₀ Determination

In vitro antimalarial activity was measured using the SYBR Green I fluorescence-based assay. 32,33 ADC derivatives were evaluated for antiplasmodial activity with chloroquine and amodiaquine serving as a control. Experiments were set up in quadruplicate in 96-well plates with a 100 μL total volume per well with 2% hematocrit, 0.2% parasitemia, and drug dilutions between 0.25 and 250 nM in complete culture medium described above. The plates were incubated at 37° C. in low-oxygen conditions described previously for 72 hours, at which point 100 μL of fluorescent dye-detergent mixture (0.2 μL SYBR Green I: 1 mL lysing buffer) was added and the plates incubated in the dark at room temperature for one hour. After incubation with the fluorescent dye, a 96-well plate reader with excitation wavelength set at 497 nm and emission at 520 nm, was used to measure fluorescence of each well. Fluorescence readings were plotted as a function of drug concentration, and curve fitting by nonlinear regression analysis (Graphpad Prism software package) gave calculated drug concentration resulting in a 50% reduction in fluorescence compared to drug-free controls (50% inhibitory concentration, IC₅₀).

e. In Vivo Efficacy Against Murine Malaria

The in vivo efficacy of selected ADC derivatives was determined against blood-stage Plasmodium yoelii using a modified 4-day test. Female mice (Charles River Laboratories) were infected intravenously with 35,000 P. yoelii parasitized erythrocytes from a donor animal (Kenya MR4 MRA-428). One day after inoculation, drugs (dissolved in PEG-400, chloroquine as positive control) were administered by oral gavage once daily for 4 successive days. Five days after inoculation, percent parasitemia was calculated by examination of Giemsa-stained blood smears. The ED₅₀ is defined as the effective drug dose that reduced parasitemia relative to no drug control values by 50% and it was calculated from the dose-response curve by nonlinear regression analysis.

f. HepG2 Cytotoxicity Assay

Final compounds were assessed for mammalian cytotoxicity using an immortalized human liver carcinoma cell line (HepG2) using previously described methods.¹ In short, final compounds were prepared in DMSO as 10 mM stock solutions. Human hepatocarcinoma (HepG2) cells were cultured in RPMI-1640 medium containing 10% fetal bovine serum at 37° C. in a humidified 5% CO₂ atmosphere. To 96-well flat-bottomed tissue culture plates, HepG2 cells were added at 2×10 4 density with an additional 160 μL of complete culture media per well and were incubated overnight at 37° C. to allow for adherence. Compound stocks aliquots were applied as 40 μL solutions in complete media to each well in a serial dilution series that ranged from 200-0.2 μL as duplicates. A 10 mM DMSO stock of mefloquine was used as a positive control. After drug-treated plates were incubated in 5% CO₂ atmosphere at 37° C. for 24 h, they were aspirated and 200 μL of complete media was added to each well for an additional 24 h incubation in same conditions as previously mentioned. 20 μL of resazurin (Alamar Blue) in PBS buffer was added to each well to a final concentration of 10 μM, and the plates were incubated for 3 h. Fluorescence was measured at 560 nm excitation and 590 nm emission bands using a Spectramax iD3 plate reader. Fluorescence output values were normalized to the untreated control wells and plotted against the logarithm of drug concentration. Cytotoxicity (CC₅₀) was determined for each compound by fitting this data to a variable slope nonlinear regression curve using Graphpad Prism software (v. 9). See Zhi-Jun et al. (1997) Journal of Immunological Methods 210(1): 25-39 and https://doi.org/10.1016/S0022-1759 (97) 00171-3.

g. Pharmacokinetic Study in Mice

The title compound ADC-028, Example No. 18, was selected for pharmacokinetic analysis in mice at a dose of 5 mg/kg performed at ChemPartner in Shanghai, China. Three groups of three male CF1 mice (JH Laboratory Animal) were administer the drug in PEG-400 at 5.0 mg/kg by oral gavage. At the following time points: 0.25, 0.5, 1, 2, 4, 8, 24, and 48 hr post dose administration a single group of mice were manually restrained and approximately 110 μL of blood were taken from the animals via facial vein for semi-serial bleeding into K 2 EDTA tubes. Samples were put on ice and centrifuged (2000 G, 5 min at under 4° C.) within 15 minutes of collection. An aliquot of 3 μL sample was added to 200 μL internal standard (Diclofenac, 40 ng/mL) in ACN. The mixture was vortexed for 1 m, and centrifuged at 5800 rpm for 10 min. 100 supernatant was transferred to a new tube. 0.5 μL of solvent was injected into LC-MS/MS and ran on a Waters ACQUITY UPLC HSS T3 (2.1×50 mm, 1.8 μm) column. Pharmacokinetic analysis as a best-fit curve was prepared from drug concentration in plasma as a function of time using WinNonlin software (Pharsight—Mountain View, CA). The exposure (AUCₗₐₛₜ), half-life (T_{1/2}), maximum concentration (Cₘₐₓ) and time of maximum concentration (Tₘₐₓ) will be determined form data. Goodness-of-fit was assessed by the r 2 (linear regression coefficient) of the drug concentration on the terminal phase.

3. Evaluation of Antiplasmodial Activity

Each of the ADC derivatives was tested for antiplasmodial activity against the drug sensitive D6 clone of P. falciparum as well as one or more of the following multidrug resistant strains: Dd2 or 7G8. The method used for antiplasmodial testing is known as the SyBr Green assay and it was described by us in 2004 (Smilkstein et al., Antimicrob Agents Chemother 2004, 48 (5), 1803-1806). Comparative in vitro IC₅₀ values are reported for each ADC derivative and these values appear in Table 1. (Note: IC₅₀ values represent the concentration of drug that is required to block parasite replication by 50% relative to control conditions without addition of test agent. As shown in the table, strain Dd2, a multidrug resistant strain from Southeast Asia, exhibits roughly 9-fold resistance to chloroquine. Cross resistance in the Dd2 strain to the ADC molecules is much reduced or completely eliminated. For example, the IC₅₀ value for ADC-009 is ˜13 nM vs. drug sensitive D6 and also for multidrug (MDR) resistant Dd2. Equipotency vs. D6 and Dd2 strains is also observed for ADC-008, ADC-010, and ADC-014 while modest cross resistance, e.g., ˜2- to 3-fold, is observed for ADC-013, ADC-017, ADC-018, ADC-020, ADC-021, and ADC-024. Without wishing to be bound by theory, these results demonstrate that the more rigid 4-amino side chain of the ADC derivatives is key to overcoming high level chloroquine resistance in the Dd2 strain.

TABLE 1

| | | | ED₅₀, ED₉₀, | |
|---|---|---|---|---|
| | IC₅₀ (nM) vs | IC₅₀ (nM) vs | NRD (mg/kg/d) | T_{1/2} (min), CIᵢₙₜ |
| ADC | D6 | Dd2 | vs Py | (mL/min/kg) |
| 008 | 16.1 | 15.9 | | 13.92, 391.9 |
| 009 | 13.2 | 12.7 | 1.5, 8.4, ND | 1.86, 2936 |
| 010 | 13.3 | 14.0 | | 1.44, 3784 |
| 011 | 21.8 | 65.8 | | |
| 013 | 39.2 | 84.4 | | |
| 014 | 69.8 | 66.0 | | |
| 015 | 10.2 | 75.2 | | |
| 017 | 5.18 | 9.17 | 4.0, 7.6, ND | ∞, 0.00 |
| 018 | 43.3 | 110.5 | | |
| 020 | 4.56 | 13.4 | 1.8, 13.4, ND | 11.0, 496.7 |
| 021 | 4.94 | 11.6 | 1.1, 4.1, ND | 44.1, 123.6 |
| 025 | 10.3 | 19.2 | | |
| 026 | 9.25 | 19.3 | | 70.0, 78.0 |
| 027 | 9.03 | 20.4 | | 42.1, 129.7 |
| 028 | 7.90 | 17.4 | 2.3, 2.5, 16 | 48.2, 113.3 |
| 029 | 13.8 | 19.0 | | 55.5, 98.3 |
| 030 | 9.79 | 17.8 | — | 13.92, 391.9 |
| CQ | | | | |
| AQ | | | | |

4. Cytotoxicity Data—ADC vs. HepG2 (CC₅₀/NM)

Cytotoxicity data was generated using the methods detailed herein above and is summarized in Table 2 below.

TABLE 2

| Example No. | ID | CC₅₀ v HepG2 (nM) |
|---|---|---|
| 1 | ADC-008 | >20.0 × 10⁴ |
| 2 | ADC-009 | >20.0 × 10⁴ |
| 3 | ADC-010 | 18.5 × 10⁴ |
| 4 | ADC-012 | >20.0 × 10⁴ |
| 5 | ADC-011 | 5.4 × 10⁴ |
| 6 | ADC-013 | >20.0 × 10⁴ |
| 7 | ADC-014 | >20.0 × 10⁴ |
| 9 | ADC-015 | 5.8 × 10⁴ |
| 10 | ADC-017 | >20.0 × 10⁴ |
| 11 | ADC-018 | >20.0 × 10⁴ |
| 12 | ADC-020 | 11.1 × 10⁴ |
| 13 | ADC-021 | >20.0 × 10⁴ |
| 14 | ADC-024 | >20.0 × 10⁴ |
| 15 | ADC-025 | 4.8 × 10⁴ |
| 16 | ADC-026 | >20.0 × 10⁴ |
| 17 | ADC-027 | >20.0 × 10⁴ |
| 18 | ADC-028 | >20.0 × 10⁴ |
| 19 | ADC-029 | >20.0 × 10⁴ |
| 20 | ADC-030 | >20.0 × 10⁴ |

5. Pharmacokinetic Study for Example No. 18, ADC-028

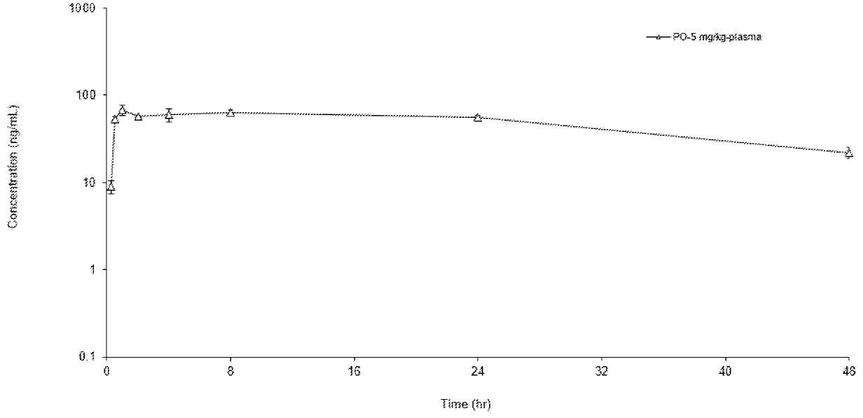

Pharmacokinetic data was generated using the protocols detailed herein above and is summarized in Table 3 below. See also FIG. 1. Notes: F=(AUC_{INF-PO}/mean AUC_{INF-IV})/(Dose_{PO}/Dose_{IV})*100%, AUClast was alternatively used for F calculation when AUC_{INF} was not available or beyond 120% of AUCₗₐₛₜ; ADC-028 was prepared in 100% PEG400 to yield clear solution at 0.5 mg/mL for PO dosing; PK parameters were estimated by non-compartmental model using WinNonlin 8.2; If the adjusted rsq (linear regression coefficient of the concentration value on the terminal phase) is less than 0.9, T½ might not be accurately estimated.

TABLE 3
Individual and mean plasma concentration-time data of
ADC-028 after a PO dose at 5 mg/kg in male CD1 mice

| Dose | Dose | Sampling time | Concentration (ng/mL) | | | Mean | | |
|---|---|---|---|---|---|---|---|---|
| (mg/kg) | route | (hr) | Individual | | | (ng/mL) | SD | CV(%) |
| 5 | PO | Pre-dose | BQL | BQL | BQL | BQL | NA | NA |

| | | | | | | | |
|---|---|---|---|---|---|---|---|
| LLOQ = 1 ng/mL | 0.25 | 8.48 | 7.86 | 10.8 | 9.05 | 1.55 | 17.1 |
| BQL < LLOQ | 0.5 | 54.6 | 47.9 | 57.0 | 53.2 | 4.72 | 8.87 |
| | 1 | 59.3 | 66.6 | 77.1 | 67.7 | 8.95 | 13.2 |
| | 2 | 56.1 | 59.3 | 57.4 | 57.6 | 1.61 | 2.79 |
| | 4 | 48.9 | 70.1 | 60.9 | 60.0 | 10.6 | 17.7 |
| | 8 | 58.6 | 67.4 | 65.5 | 63.8 | 4.63 | 7.25 |
| | 24 | 57.4 | 58.1 | 52.6 | 56.0 | 2.99 | 5.34 |
| | 48 | 18.3 | 24.5 | 23.1 | 22.0 | 3.25 | 14.8 |

| PK parameters | Unit | Estimated Value |
|---|---|---|
| Rsq_adjusted | | 0.829 |
| Tₘₐₓ | hr | 1.00 |
| Cₘₐₓ | ng/mL | 67.7 |
| No_points_lambda_z | | 3 |
| T_{1/2} | hr | 25.1 |
| AUCₗₐₛₜ | hr*ng/mL | 2362 |
| AUC_{INF} | hr*ng/mL | 3156 |
| MRT_{Iast} | hr | 19.3 |
| MRT_{INF} | hr | 35.6 |

6. Prophetic Exemplary Compounds

Additional compounds within the scope of the compounds, pharmaceutical formulations, and methods of treatment include the following:

### a. Example No. 21-7-chloro-N-(4-(4-isopropylpiperazin-1-yl)phenyl)quinolin-4-amine

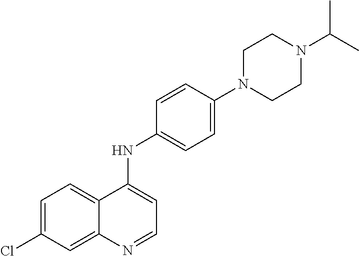 [SMILES: CC(C)N1CCN(c2ccc(Nc3ccnc4cc(Cl)ccc34)cc2)CC1]

### b. Example No. 22-7-chloro-N-(4-(1-(cyclopropylmethyl)piperidin-4-yl)phenyl)quinolin-4-amine

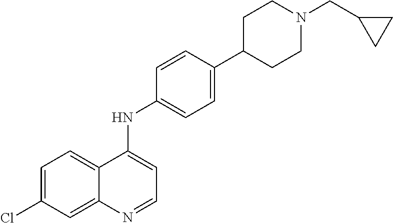 [SMILES: Clc1ccc2c(Nc3ccc(C4CCN(CC5CC5)CC4)cc3)ccnc2c1]

### c. Example No. 23-7-chloro-N-(4-(4-(cyclopropylmethyl)piperazin-1-yl)phenyl)quinolin-4-amine

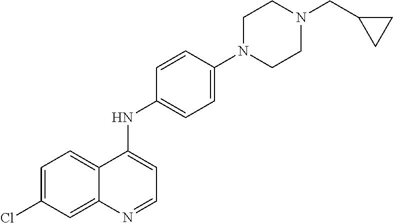 [SMILES: Clc1ccc2c(Nc3ccc(N4CCN(CC5CC5)CC4)cc3)ccnc2c1]

### d. Example No. 24-1-(4-(4-((7-chloroquinolin-4-yl)amino)phenyl)piperidin-1-yl)-2,2-dimethylpropan-1-one

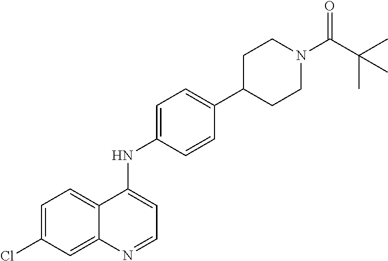 [SMILES: CC(C)(C)C(=O)N1CCC(c2ccc(Nc3ccnc4cc(Cl)ccc34)cc2)CC1]

### e. Example No. 25-N-((1R,3R,5R,7R)-adamantan-2-yl)-4-(4-((7-chloroquinolin-4-yl)amino)phenylpiperazine-1-carboxamide

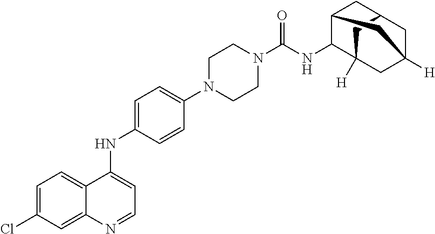 [SMILES: O=C(NC1[C@H]2C[C@H]3C[C@H](C2)C[C@@H]1C3)N1CCN(c2ccc(Nc3ccnc4cc(Cl)ccc34)cc2)CC1]

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed:

1. A compound of Formula (I), or a pharmaceutically acceptable salt thereof: 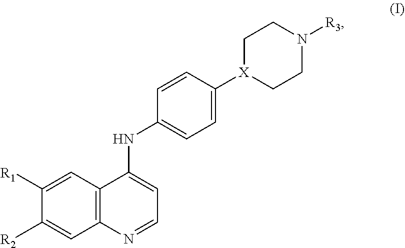
wherein:
R₁ is selected from H and halogen;
R₂ is selected from H, halogen, and halomethyl;
R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and
X is selected from N and C;
with the proviso that, when R₁ is H, R₂ is not H;
with the proviso that, when R₂ is H, R₁ is not H; and
with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; 7-chloro-N-(4-(1-methylpiperidin-4-yl)phenyl)quinolin-4-amine; 6-bromo-N-(4-(4-methylpiperazin-1-yl)phenyl)quinolin-4-amine; or 6-bromo-N-(4-(4-ethylpiperazin-1-yl)phenyl)quinolin-4-amine; or a pharmaceutically acceptable salt thereof.

2. The compound of claim 1, wherein X is N; or a pharmaceutically acceptable salt thereof.

3. The compound of claim 1, wherein X is C; or a pharmaceutically acceptable salt thereof.

4. The compound of claim 1, wherein R₃ is selected from H, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; or a pharmaceutically acceptable salt thereof.

5. The compound of claim 1, wherein R₃ is H; or a pharmaceutically acceptable salt thereof.

6. The compound of claim 1, wherein R₃ is —C(═O)—C₁-C₇ straight or branched alkyl; or a pharmaceutically acceptable salt thereof.

7. The compound of claim 1, wherein R₃ is selected from C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; or a pharmaceutically acceptable salt thereof.

8. The compound of claim 1, or a pharmaceutically acceptable salt thereof, having a Formula (I): 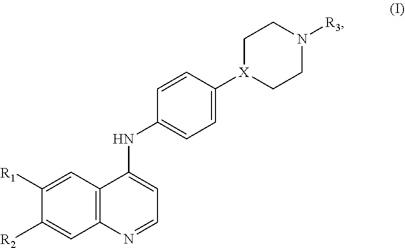
wherein:
R₁ is selected from H and halogen;
R₂ is selected from H, halogen, and halomethyl;
R₃ is selected from H, C₁-C₇ straight or branched alkyl, —C(═O)—C₁-C₇ straight or branched alkyl, —C(═O)—NH—C₁-C₇ straight or branched alkyl, C₃-C₁₀ cycloalkyl, —CH₂—C₃-C₁₀ cycloalkyl, —C(═O)—C₃-C₁₀ cycloalkyl, —C(═O)—NH—C₃-C₁₀ cycloalkyl, and benzyl; and
X is selected from N and C;
with the proviso that one or more selected from:
(a) X is C and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl;
(b) R₁ is halogen and R₂ is selected from H and halomethyl; and
(c) R₂ is halomethyl and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl.

9. The compound of claim 8, or a pharmaceutically acceptable salt thereof, having a Formula (VI): 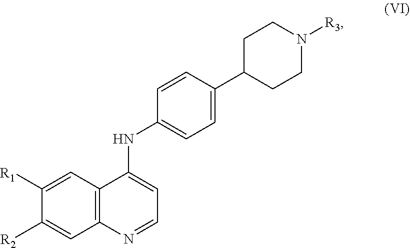
wherein:
R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl.

10. The compound of claim 8, wherein R₁ is halogen and R₂ is selected from H and halomethyl; or a pharmaceutically acceptable salt thereof.

11. The compound of claim 8, wherein R₂ is halomethyl and R₃ is selected from H, —C(═O)—C₁-C₇ straight or branched alkyl, C₃-C₁₀ cycloalkyl, —CH₂—C₃-C₁₀ cycloalkyl, —C(═O)—C₃-C₁₀ cycloalkyl, and —C(═O)—NH—C₃-C₁₀ cycloalkyl; or a pharmaceutically acceptable salt thereof.

12. The compound of claim 1, wherein R₃ is selected from H, C₁-C₇ straight or branched alkyl, C₃-C₆ cycloalkyl, —CH₂—C₃-C₆ cycloalkyl, and benzyl; or a pharmaceutically acceptable salt thereof.

13. The compound of claim 1, or a pharmaceutically acceptable salt thereof, having Formula (II): 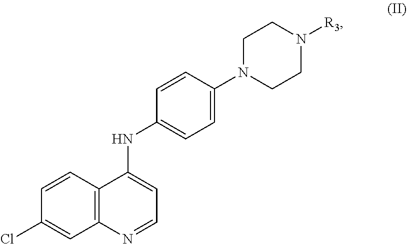
wherein R₃ is selected from H, C₁-C₇ straight or branched alkyl, C₃-C₆ cycloalkyl, —CH₂—C₃-C₆ cycloalkyl, and benzyl;
with the proviso that the compound is not N-(4-(4-benzylpiperazin-1-yl)phenyl)-7-chloroquinolin-4-amine; N-(4-(4-butylpiperazin-1-yl)phenyl)-7-chloroquinolin-4-amine; 7-chloro-N-(4-(4-methylpiperazin-1-yl)phenyl)quinolin-4-amine; or 7-chloro-N-(4-(1-methylpiperidin-4-yl)phenyl)quinolin-4-amine; or a pharmaceutically acceptable salt thereof.

14. The compound of claim 1, or a pharmaceutically acceptable salt thereof, having Formula (III): 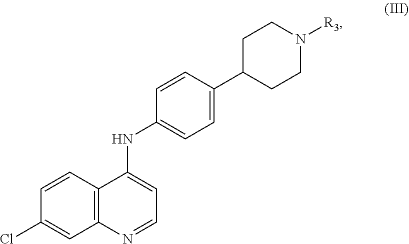
wherein R₃ is selected from H and C₂-C₇ straight or branched alkyl; or a pharmaceutically acceptable salt thereof.

15. The compound of claim 1, having Formula (IV): 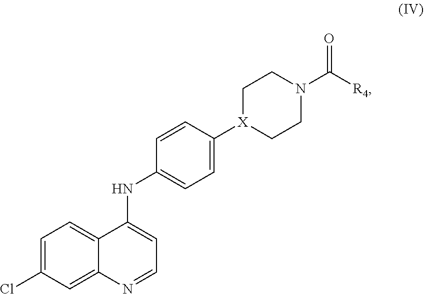
wherein: X is selected group of N and C; and R₄ is selected from H, C₁-C₇ straight or branched alkyl, and C₃-C₁₀ cycloalkyl; or a pharmaceutically acceptable salt thereof.

16. The compound of claim 1, having Formula (V): 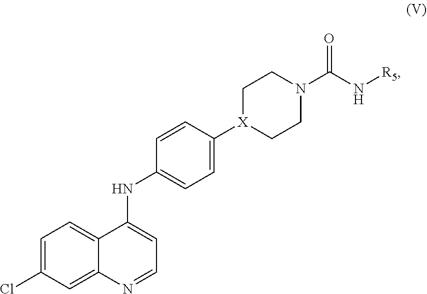
wherein: X is selected from N and C; and R₅ is selected from H, C₁-C₇ straight or branched alkyl, and C₃-C₁₀ cycloalkyl; or a pharmaceutically acceptable salt thereof.

17. The compound of claim 1, selected from: 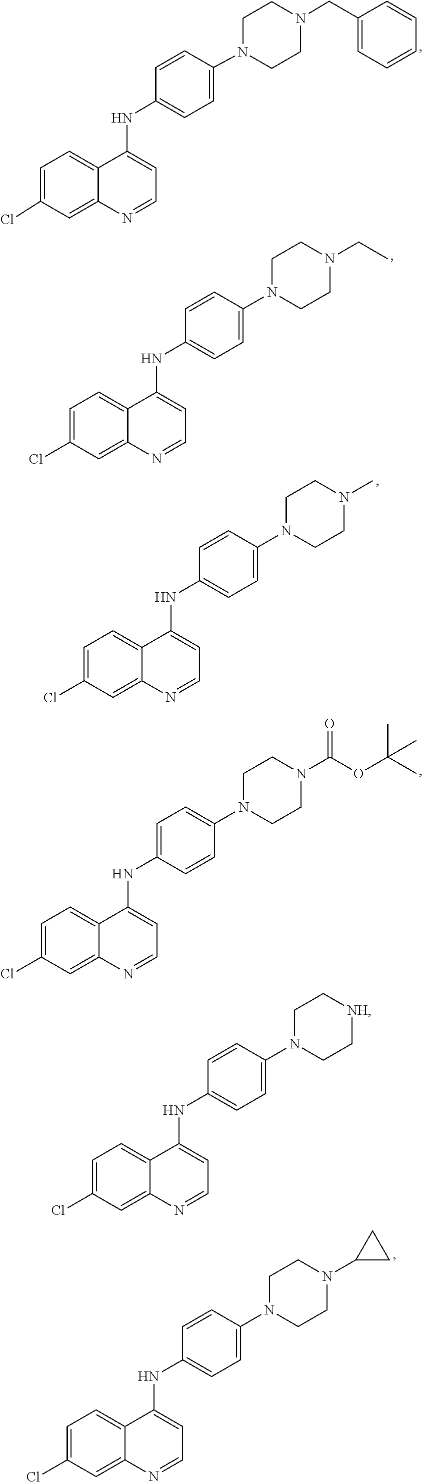 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1 ; Clc1ccc2c(Nc3ccc(N4CCN(C5CC5)CC4)cc3)ccnc2c1] 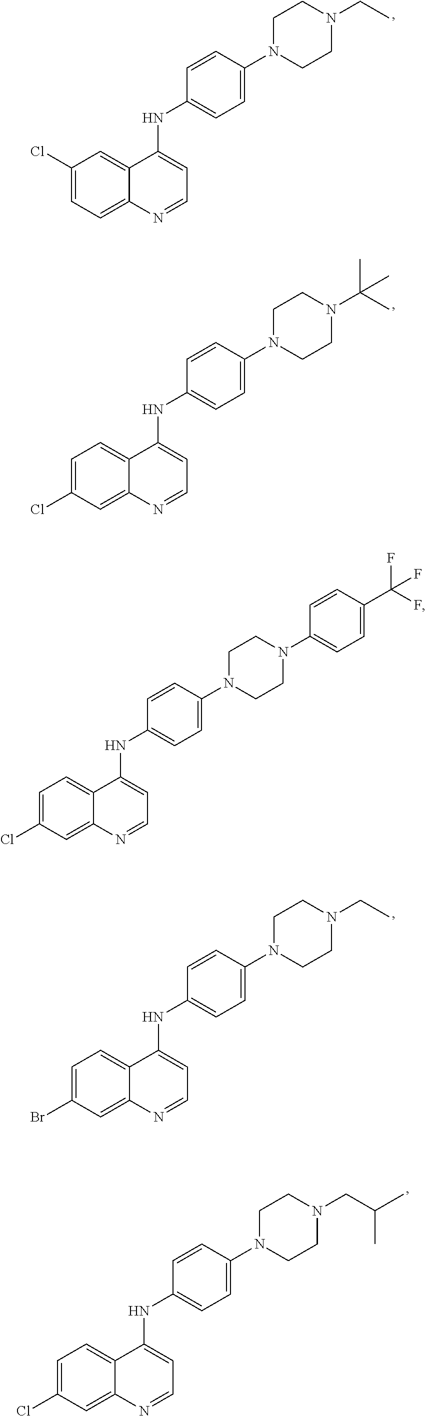 [SMILES: CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1 ; CCN1CCN(c2ccc(Nc3ccnc4cc(Br)ccc34)cc2)CC1 ; CC(C)CN1CCN(c2ccc(Nc3ccnc4cc(Cl)ccc34)cc2)CC1] 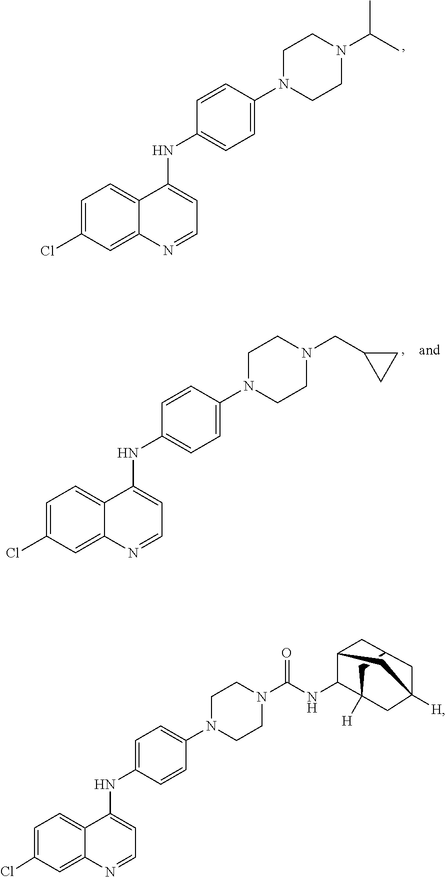
or a pharmaceutically acceptable salt thereof.

18. A pharmaceutical composition comprising a pharmaceutically acceptable carrier or excipient and a pharmaceutically effective amount of the compound of claim 1; or a pharmaceutically acceptable salt thereof.

19. A method of treating malaria in a subject in need thereof, the method comprising administering to the subject a pharmaceutically effective amount of the compound of claim 1; or a pharmaceutically acceptable salt thereof.

20. A compound selected from: 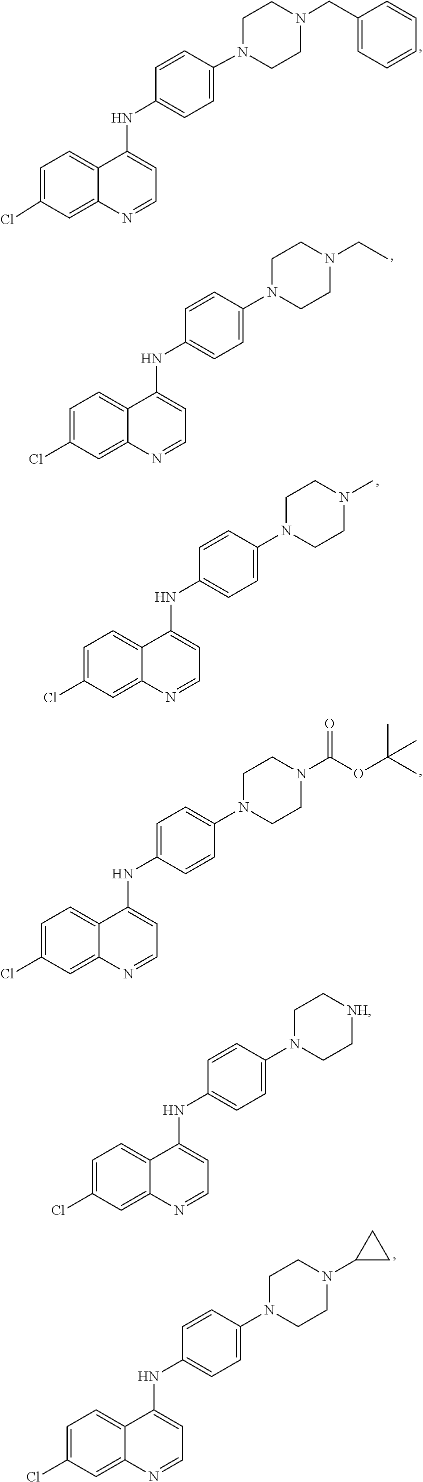 [SMILES: Clc1ccc2c(Nc3ccc(N4CCN(Cc5ccccc5)CC4)cc3)ccnc2c1 ; CCN1CCN(c2ccc(Nc3ccnc4cc(Cl)ccc34)cc2)CC1 ; CN1CCN(c2ccc(Nc3ccnc4cc(Cl)ccc34)cc2)CC1 ; CC(C)(C)OC(=O)N1CCN(c2ccc(Nc3ccnc4cc(Cl)ccc34)cc2)CC1 ; Clc1ccc2c(Nc3ccc(N4CCNCC4)cc3)ccnc2c1 ; Clc1ccc2c(Nc3ccc(N4CCN(C5CC5)CC4)cc3)ccnc2c1] 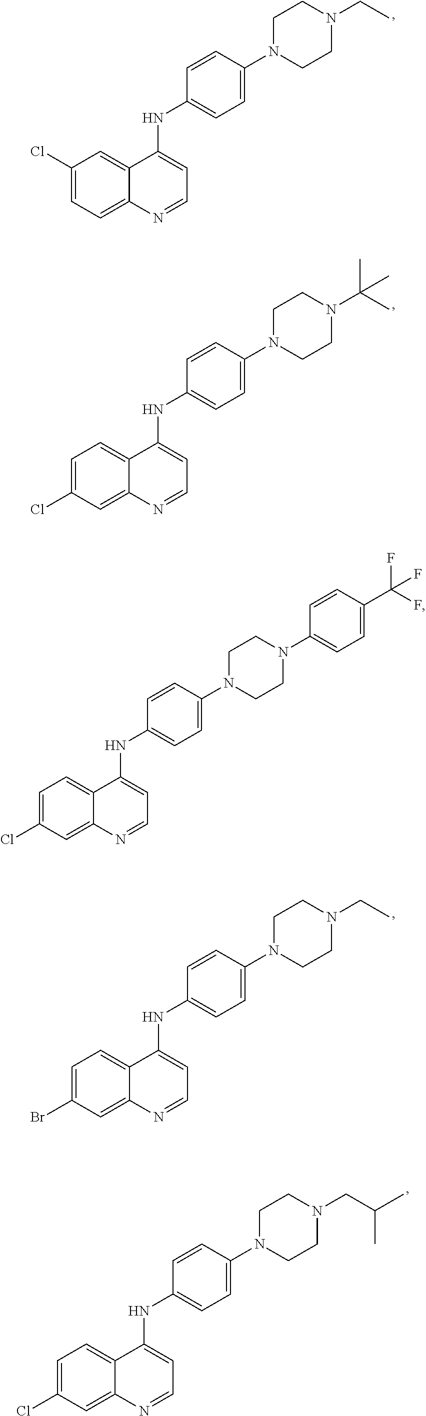 [SMILES: CCN1CCN(c2ccc(Nc3ccnc4ccc(Cl)cc34)cc2)CC1 ; CC(C)(C)N1CCN(c2ccc(Nc3ccnc4cc(Cl)ccc34)cc2)CC1 ; FC(F)(F)c1ccc(N2CCN(c3ccc(Nc4ccnc5cc(Cl)ccc45)cc3)CC2)cc1 ; CCN1CCN(c2ccc(Nc3ccnc4cc(Br)ccc34)cc2)CC1 ; CC(C)CN1CCN(c2ccc(Nc3ccnc4cc(Cl)ccc34)cc2)CC1] 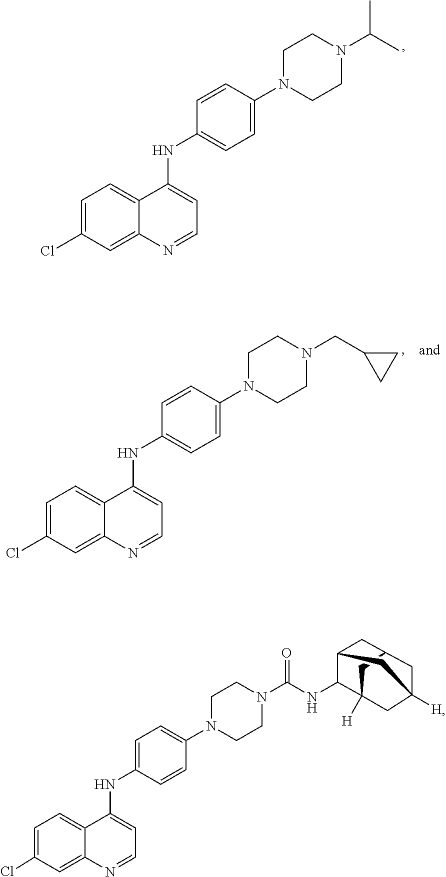
or a pharmaceutically acceptable salt thereof.
